(19) United States
(12) Patent Application Publication
(10) Pub. No.: US 2025/0188515 A1
(43) Pub. Date: Jun. 12, 2025

(54) DEPROTECTION-COUNTING PROBES FOR DETECTING AND QUANTIFYING SINGLE MOLECULAR ANALYTES

(71) Applicant: The Regents of the University of Michigan, Ann Arbor, MI (US)

(72) Inventor: Alexander Edmund Johnson-Buck, Ann Arbor, MI (US)

(21) Appl. No.: 18/580,317

(22) PCT Filed: Jul. 14, 2022
(86) PCT No.: PCT/US22/73707

§ 371 Date: Jul. 19, 2024

Related U.S. Application Data
(60) Provisional application No. 63/224,984, filed on Jul. 23, 2021.

(51) Int. Cl. C12Q 1/6818; C12Q 1/6834; C12Q 1/6876; G01N 21/64; G01N 33/543; G01N 33/68

(52) U.S. Cl. CPC C12Q 1/6818; C12Q 1/6834; C12Q 1/6876; G01N 21/6428; G01N 33/54313; G01N 33/6803; G01N 2021/6432; G01N 2021/6441

(57) ABSTRACT

A molecular architecture and method to better distinguish between specific and nonspecific binding events using molecular probes that possess an internal record of repeated binding of probes to the same analyte molecule, permitting the high-sensitivity, high-specificity detection of analytes (e.g., nucleic acids, proteins, or other biomolecules) is provided. The repeated binding of probes to the same analyte molecule (or to a probe bound to the analyte molecule) results in an accumulation of signal (e.g., fluorescence or chemiluminescence) dependent on the number and kinetics of probe binding events, yielding increased confidence in the presence of the analyte molecule as the number of independent binding events increases.

### CROSS-REFERENCE TO RELATED APPLICATIONS

This application claims priority to U.S. provisional application Ser. No. 63/224,984, filed on Jul. 23, 2021, which is incorporated herein by reference in its entirety.

### SEQUENCE LISTING

The computer readable sequence listing filed herewith, titled “UM-39715-601_SQL”, created Jul. 13, 2022, having a file size of 46,117 bytes, is hereby incorporated by reference in its entirety.

### BACKGROUND

Molecular affinity probes such as nucleic acid oligonucleotides, aptamers, antibodies and antibody fragments are often used to identify and quantify target biomarkers of interest, such as DNA sequences, RNA sequences, proteins, or covalent modifications thereof, in biological mixtures. However, a shortcoming of the use of such probes is that they typically exhibit significant amounts of unintended (or nonspecific) binding to surfaces and other components in the biological mixture. This produces background signal that makes it difficult or impossible to detect low (e.g., sub-picomolar or sub-femtomolar) concentrations of the target of interest, since it becomes extremely difficult or technically impractical to distinguish the small amount of signal arising from the intended (specific) binding from the significant signal arising from unintended (nonspecific) binding.

Thus, there is a need for improved systems, methods and kits for providing high-sensitivity and high-specificity detection of analytes, particularly at low concentrations.

### SUMMARY

The present disclosure provides a generalized molecular architecture and method to better distinguish between specific and nonspecific binding using molecular probes that possess an internal record of repeated binding of probes to the same analyte molecule (or to a probe bound to the analyte molecule), permitting the high-sensitivity, high-specificity detection of analytes (e.g., nucleic acids, proteins, or other biomolecules). The repeated binding of probes to the same analyte molecule (or to a probe bound to the analyte molecule) results in an accumulation of signal (e.g., fluorescence or chemiluminescence) dependent on the number and kinetics of probe binding events, yielding increased confidence in the presence of the analyte molecule as the number of independent binding events increases. Since nonspecific binding is unlikely to occur repeatedly in the same location with the same affinity or kinetics as specific binding, the accumulation of signal arising from repeated probe binding to the same analyte molecule (or to a probe bound to the analyte molecule) constitutes a characteristic signature of the presence of the analyte molecule.

The present disclosure provides systems, kits and methods for detection and quantification of molecular analytes. In one embodiment, the present disclosure provides a system of detecting an analyte in a sample. The system comprises a plurality of first probes, each first probe comprising a signal region, wherein the signal region comprises a plurality of suppressor binding regions; a plurality of suppressors, wherein each suppressor comprises a first probe binding region that is able to bind to the suppressor binding region of the first probe such that at least one suppressor is associated with substantially all of the suppressor binding regions of the first probes; a plurality of analyte binding partners, wherein each analyte binding partner comprises a first analyte binding region having affinity to the analyte, and wherein each of the analyte binding partners are directly or indirectly associated with at least one of the first probes; a plurality of second probes, each second probe comprising a second analyte binding region and a suppressor interacting region, wherein the second analyte binding region has affinity to the analyte, wherein the suppressor interacting region is able to bind to one or more of the suppressors; wherein the second analyte binding region of the second probe is able to bind the analyte while the analyte is bound to the first analyte binding region of the analyte binding partner; wherein the interaction of the first analyte binding region to the analyte possesses a rate constant of dissociation that is slower than the rate constant of dissociation between the second analyte binding region and the analyte; wherein when one of the second probes binds to the analyte while the analyte is bound to one of the analyte binding partners, such second probe also binds to one or more of the suppressors such that such one or more suppressors bound to the second probe will be removed from the first probe upon disassociation of the second probe from the analyte; and a plurality of label probes, each label probe having an affinity to the signal region of the first probe, wherein upon removal of one or more suppressors, one or more label probes are able to bind to the signal region of the first probe thereby permitting detection of the analyte.

In another embodiment, a kit comprising components for using the above described system is provided. The kit comprises: a first component comprising a plurality of first probes, each first probe comprising a first analyte binding region and a signal region, wherein the first analyte binding region has affinity to the analyte, wherein the first analyte binding region and signal region are either directly associated as part of a common molecule or indirectly associated as two distinct molecules; a second component comprising a plurality of label probes, wherein each label probe is able to associate with the signal region; a third component comprising a plurality of suppressors, wherein each suppressor is able to associate with the signal region; wherein the suppressors prevent detection of the label probes when both are associated with the signal region or where the suppressors prevent association of the label probes with the signal region; and a fourth component comprising a plurality of second probes, each second probe comprising a second analyte binding region and a suppressor interacting region, wherein the second analyte binding region has affinity to the analyte, wherein the suppressor interacting region is indirectly or directly capable of binding to, removing, or degrading one or more of the suppressors, and wherein the interaction of the first analyte binding region to the analyte possesses a rate constant of dissociation that is slower than the rate constant of dissociation between the second analyte binding region and the analyte.

In yet another embodiment, a method for detecting an analyte in a sample using the above described kit is provided. The method comprises: mixing a portion of the sample with the first component and third component to form a first reaction mixture, wherein the suppressors of the third component are bound to the signal region of the plurality of first probes of the first component; incubating the first reaction mixture under conditions sufficient for the analyte to bind to the first analyte binding region; mixing the first reaction mixture following step (b) with the fourth component to yield a second reaction mixture; incubating the second reaction mixture under conditions sufficient for the second probe to bind to and dissociate from the analyte and remove one or more suppressors from the signal region of the plurality of first probe or degrade one or more suppressors; mixing the second reaction mixture following step (d) with the second component to yield a third reaction mixture; incubating the third reaction mixture under conditions sufficient to permit the label probe to associate with the signal regions of first probes that have had one or more suppressors removed or degraded in step (d); and measuring a signal generated from the label probes associated with the signal regions of the first probe to determine the presence of the analyte in the sample.

### BRIEF DESCRIPTION OF THE DRAWINGS

FIG. 1 depicts an exemplary embodiment of the present disclosure.

FIG. 2 depicts an exemplary embodiment of the present disclosure.

FIG. 3 depicts the exemplary analyte detection system of Example 1. FIG. 3 discloses, in order of appearance, SEQ ID NO: 1 (which includes 5 copies of SEQ ID NO: 2), SEQ ID NO: 3 (which includes SEQ ID NO: 4), SEQ ID NO: 6 (which includes, from 5′ to 3′, SEQ ID NO: 7, SEQ ID NO: 8 and SEQ ID NO: 9), SEQ ID NO: 10, SEQ ID NO: 5, SEQ ID NO: 3, SEQ ID NO: 3, SEQ ID NO: 3, SEQ ID NO: 3, SEQ ID NO: 3, and SEQ ID NO: 1.

FIG. 4 depicts the exemplary method for analyte detection of Example 1. In this exemplary embodiment, detection of a biotinylated DNA sequence using a first probe including a streptavidin-gold nanoparticle is shown where the first probe is bound to a biotinylated coverslip surface. Note that while a first probe complex with one gold nanoparticle and one signal region is shown, each gold nanoparticle can be part of a first probe that includes one or more, e.g. 10 or more signal regions, due to multiple binding sites for the biotin-labeled DNA oligonucleotide 111. FIG. 4 discloses, in order of appearance, SEQ ID NO: 5, SEQ ID NO: 3, SEQ ID NO: 3, SEQ ID NO: 3, SEQ ID NO: 3, SEQ ID NO: 3, SEQ ID NO: 1, SEQ ID NO: 6, SEQ ID NO: 5, SEQ ID NO: 6, SEQ ID NO: 3, SEQ ID NO: 3, SEQ ID NO: 3, SEQ ID NO: 3, SEQ ID NO: 3, SEQ ID NO: 1, SEQ ID NO: 6, SEQ ID NO: 3, SEQ ID NO: 5, SEQ ID NO: 3, SEQ ID NO: 3, SEQ ID NO: 3, SEQ ID NO: 3, SEQ ID NO: 1, SEQ ID NO: 5, SEQ ID NO: 1, SEQ ID NO: 10, SEQ ID NO: 5, SEQ ID NO: 10, SEQ ID NO: 10, SEQ ID NO: 1, SEQ ID NO: 10, SEQ ID NO: 10, and SEQ ID NO: 10.

FIG. 5 depicts a TIRF microscopy image of the coverslip from Example 1 in the presence of the analyte oligonucleotide (left) and a TIRF microscopy image of the coverslip in the absence of the analyte oligonucleotide (right).

FIG. 6 depicts step photobleaching analysis data from a fluorescent punctum arising from an individual first probe for Example 1 in the presence of the analyte oligonucleotide (left) and step photobleaching analysis data from a fluorescent punctum arising from an individual first probe for Example 1 in the absence of the analyte oligonucleotide (right).

FIG. 7 depicts TIRF microscopy images after various incubation times of the second probe with or without the analyte in for Example 2.

FIG. 8A depicts quantification for the experiment in Example 2 using varying thresholds for different incubation times. Error bars represent 1 standard deviation for 3 measurements from separate fields of view in the same sample well.

FIG. 8B depicts quantification for the experiment in Example 2 using a single 30,000 arbitrary unit threshold for different incubation times. Error bars represent 1 standard deviation for 3 measurements from separate fields of view in the same sample well.

FIG. 9 depicts the number of accepted particles per field of view for Example 3. Each data point is the average of 25 fields of view. Error bars represent 1 standard deviation.

FIG. 10 depicts the exemplary method for analyte detection of Example 4. In this exemplary embodiment, detection of a protein using a first probe including a streptavidin-gold nanoparticle is shown where the first probe is bound to a biotinylated coverslip surface. Note that while a first probe complex with one gold nanoparticle and one signal region is shown, each gold nanoparticle can be part of a first probe that includes one or more, e.g. 10 or more signal regions, due to multiple binding sites for the biotin-labeled DNA oligonucleotide 111. Note also that while a first probe complex with one first analyte binding region is shown, each gold nanoparticle can be part of a first probe that includes one or more, e.g., 10 or more first analyte binding regions, due to multiple binding sites for the biotin-labeled capture antibody. FIG. 10 discloses, in order of appearance, SEQ ID NO: 3, SEQ ID NO: 3, SEQ ID NO: 3, SEQ ID NO: 3, SEQ ID NO: 3, SEQ ID NO: 1, SEQ ID NO: 11 (which includes, from 5′ to 3′, SEQ ID NO: 8 and SEQ ID NO: 9), SEQ ID NO: 11, SEQ ID NO: 3, SEQ ID NO: 3, SEQ ID NO: 3, SEQ ID NO: 3, SEQ ID NO: 3, SEQ ID NO: 1, SEQ ID NO: 11, SEQ ID NO: 3, SEQ ID NO: 3, SEQ ID NO: 3, SEQ ID NO: 3, SEQ ID NO: 3, SEQ ID NO: 1, SEQ ID NO: 1, SEQ ID NO: 10, SEQ ID NO: 10, SEQ ID NO: 10, SEQ ID NO: 10, SEQ ID NO: 1, SEQ ID NO: 10, and SEQ ID NO: 10.

FIG. 11A depicts a TIRF microscopy image of the coverslip in the presence of the PAI-1 analyte (left) and a TIRF microscopy image of the coverslip in the absence of the PAI-1 analyte (right).

FIG. 11B depicts quantification for the experiment in Example 4. Error bars represent 1 standard deviation of 5 independent groups of 5 fields of view in the same sample well.

FIG. 11C depicts a TIRF microscopy image of the coverslip in the presence of the TNF-alpha analyte (left) and a TIRF microscopy image of the coverslip in the absence of the TNF-alpha analyte (right).

FIG. 12 depicts an exemplary system where a capture oligonucleotide can be used to capture a non-biotinylated nucleic acid analyte. FIG. 12 discloses, in order of appearance, SEQ ID NO: 12, SEQ ID NO: 6, SEQ ID NO: 12, SEQ ID NO: 13, SEQ ID NO: 3, SEQ ID NO: 3, SEQ ID NO: 3, SEQ ID NO: 3, SEQ ID NO: 3, and SEQ ID NO: 1.

FIG. 13 depicts the exemplary system of FIG. 12 where the streptavidin-biotin interactions have been replaced with gold-thiol interactions between the solid support and gold nanoparticle and between the gold nanoparticle and both the signal region and capture oligonucleotide. FIG. 13 discloses, in order of appearance, SEQ ID NO: 6, SEQ ID NO: 12, SEQ ID NO: 14, SEQ ID NO: 3, SEQ ID NO: 3, SEQ ID NO: 3, SEQ ID NO: 3, SEQ ID NO: 3, and SEQ ID NO: 15.

FIG. 14 depicts an exemplary embodiment where a capture antibody is conjugated to a silica nanoparticle that is conjugated to a signal region and further linked to a solid support. FIG. 14 discloses, in order of appearance, SEQ ID NO: 11, SEQ ID NO: 3, SEQ ID NO: 3, SEQ ID NO: 3, SEQ ID NO: 3, SEQ ID NO: 3, and SEQ ID NO: 15.

FIG. 15 depicts an exemplary embodiment of the present disclosure using DNA origami to bind the signal region and, via capture oligonucleotide, the first analyte binding region (capture antibody conjugated to a DNA oligonucleotide). FIG. 15 discloses, in order of appearance, SEQ ID NO: 3, SEQ ID NO: 3, SEQ ID NO: 3, SEQ ID NO: 3, SEQ ID NO: 3, SEQ ID NO: 1, SEQ ID NO: 14, SEQ ID NO: 14, SEQ ID NO: 11, SEQ ID NO: 15, SEQ ID NO: 3, SEQ ID NO: 3, SEQ ID NO: 3, SEQ ID NO: 3, and SEQ ID NO: 3.

FIG. 16 depicts an exemplary embodiment of the present disclosure where a capture antibody (first analyte binding region) is immobilized directly at a solid support via streptavidin-biotin interaction. FIG. 16 discloses, in order of appearance, SEQ ID NO: 11, SEQ ID NO: 3, SEQ ID NO: 3, SEQ ID NO: 3, SEQ ID NO: 3, SEQ ID NO: 3, and SEQ ID NO: 15.

FIG. 17 depicts an exemplary embodiment of the present disclosure where the signal region includes reactive groups (azide) that can be uncovered by the suppressor interacting region, which includes an enzyme, degrading the bulky polymer suppressors blocking the reactive groups which can then be reacted to conjugate a label to the reactive groups.

FIG. 18 depicts an exemplary embodiment of the present disclosure where the first probe is not coupled to a solid support and where detection can include non-microscopic methods. FIG. 18 discloses, in order of appearance, SEQ ID NO: 12, SEQ ID NO: 14, SEQ ID NO: 3, SEQ ID NO: 3, SEQ ID NO: 3, SEQ ID NO: 3, SEQ ID NO: 3, SEQ ID NO: 15, SEQ ID NO: 6, SEQ ID NO: 12, SEQ ID NO: 6, SEQ ID NO: 14, SEQ ID NO: 15, SEQ ID NO: 3, SEQ ID NO: 3, SEQ ID NO: 3, SEQ ID NO: 3, SEQ ID NO: 3, SEQ ID NO: 6, SEQ ID NO: 3, SEQ ID NO: 6, SEQ ID NO: 12, SEQ ID NO: 14, SEQ ID NO: 15, SEQ ID NO: 3, SEQ ID NO: 3, SEQ ID NO: 3, SEQ ID NO: 3, SEQ ID NO: 6, SEQ ID NO: 12, SEQ ID NO: 14, SEQ ID NO: 15, SEQ ID NO: 10, SEQ ID NO: 6, SEQ ID NO: 12, SEQ ID NO: 14, SEQ ID NO: 15, SEQ ID NO: 10, SEQ ID NO: 10, SEQ ID NO: 10, SEQ ID NO: 10, and SEQ ID NO: 10.

FIG. 19 depicts an exemplary embodiment of the present disclosure where the removal of the suppressors dequenches fluorophores associated with the signal region to produce a signal. FIG. 19 discloses, in order of appearance, SEQ ID NO: 5, SEQ ID NO: 3, SEQ ID NO: 3, SEQ ID NO: 3, SEQ ID NO: 3, SEQ ID NO: 3, SEQ ID NO: 1, SEQ ID NO: 6, SEQ ID NO: 6, SEQ ID NO: 5, SEQ ID NO: 3, SEQ ID NO: 3, SEQ ID NO: 3, SEQ ID NO: 3, SEQ ID NO: 3, SEQ ID NO: 1, SEQ ID NO: 6, SEQ ID NO: 3, SEQ ID NO: 5, SEQ ID NO: 3, SEQ ID NO: 3, SEQ ID NO: 3, SEQ ID NO: 3, SEQ ID NO: 1, SEQ ID NO: 5, SEQ ID NO: 1.

FIG. 20 depicts an exemplary embodiment of the present disclosure where a nanoparticle is not included in the first probe and a biotinylated analyte can bind to the streptavidin and the signal region can be bound to the streptavidin. FIG. 20 discloses, in order of appearance, SEQ ID NO: 6, SEQ ID NO: 5, SEQ ID NO: 3, SEQ ID NO: 3, SEQ ID NO: 3, SEQ ID NO: 3, SEQ ID NO: 3, SEQ ID NO: 3, SEQ ID NO: 3, SEQ ID NO: 3, SEQ ID NO: 3, and SEQ ID NO: 16.

FIG. 21 depicts a TIRF microscopy image of the coverslip in the presence of the analyte (left) and a TIRF microscopy image of the coverslip in the absence of the analyte (right) in an experiment performed according to the embodiment illustrated in FIG. 20.

FIG. 22 depicts an exemplary system and method of the present disclosure where the second probe can bind to the first probe when it is bound to the analyte.

FIG. 23 depicts an exemplary system and method where the first analyte binding region includes an aptamer which can change conformation upon analyte binding to permit binding of the second probe to the first probe. FIG. 23 discloses, in order of appearance, SEQ ID NO: 18, SEQ ID NO: 3, SEQ ID NO: 3, SEQ ID NO: 3, SEQ ID NO: 3, SEQ ID NO: 3, SEQ ID NO: 1, SEQ ID NO: 18, SEQ ID NO: 3, SEQ ID NO: 3, SEQ ID NO: 3, SEQ ID NO: 3, SEQ ID NO: 3, SEQ ID NO: 1, SEQ ID NO: 19, SEQ ID NO: 18, SEQ ID NO:19, SEQ ID NO: 3, SEQ ID NO: 3, SEQ ID NO: 3, SEQ ID NO: 3, SEQ ID NO: 3, SEQ ID NO: 1, SEQ ID NO: 19, SEQ ID NO: 3, SEQ ID NO: 18, SEQ ID NO: 19, SEQ ID NO: 3, SEQ ID NO: 3, SEQ ID NO: 3, SEQ ID NO: 3, SEQ ID NO: 3, SEQ ID NO: 1, SEQ ID NO: 18, SEQ ID NO: 19, SEQ ID NO: 1, SEQ ID NO: 10, SEQ ID NO: 18, SEQ ID NO: 19, SEQ ID NO: 10, SEQ ID NO: 10, SEQ ID NO: 1, SEQ ID NO: 10, SEQ ID NO: 10, SEQ ID NO: 10.

FIG. 24 depicts an exemplary system and method for selective capture of labeled first probes using an immobilized multivalent probe.

FIG. 25 is a working example of the system and method shown in FIG. 24. Panel A shows exemplary sequence of the signal region 111 (SEQ ID NO: 20), the label probes modified to include an additional sequences that is complementary to the multivalent interaction probe (affinity component) (each label probe SEQ ID NO: 21) and the multivalent label interaction probe (SEQ ID NO: 22). Panel B depicts images captured using total internal reflection fluorescence microscopy of chambers using the exemplary system set forth in Panel A where analyte was added (left panel) and where no analyte was added (right panel). Below each image is its corresponding histogram quantifying the fluorescence intensity measured in the analyte chamber and blank chamber, respectively.

FIG. 26 is a working example of an embodiment of the present system and method using a branched signal region in the first probe versus a linear signal region. Panel A depicts an exemplary signal region oligonucleotide 111 (SEQ ID NO: 23) and an exemplary suppressor oligonucleotide (SEQ ID NO: 3). Panel B depicts a branched signal region comprising a first oligonucleotide (SEQ ID NO: 24) and a series of second oligonucleotides (SEQ ID NO: 25) that include a region complementary to the first oligonucleotide and a region complementary to the suppressor oligonucleotides (SEQ ID NO: 3). Panels C and D provide histograms quantifying the fluorescence intensity of linear signal regions versus branched signal regions of different lengths (top panels are with analyte and bottom panels are controls without analyte).

FIG. 27 is a working example using the analyte detection system described in Example 6 except that a comparison is made between a target analyte and a non-target analyte that differs by only a single nucleotide substitution. Histograms showing quantified intensity measured from samples with the target analyte (Target DNA), the non-target analyte (Single-Nucleotide Substitution (A-T), and a control with no analyte (blank). The top row of panels show intensities when a competitor probe was added to the sample versus no competitor probe (bottom row). The competitor probe is a 10 bp oligonucleotide that is complementary to the non-target analyte.

FIG. 28 is a working example involving a nucleic acid analyte where the first probe oligonucleotides (signal regions) include an analyte binding sequence that binds directly to the analyte and can bind to the analyte at multiple sites. The top panel depicts the analyte (SEQ ID NO: 26) hybridized to two signal probes (SEQ ID NO: 27) each also hybridized to four suppressor oligonucleotides (SEQ ID NO: 3). The second probe oligonucleotide (SEQ ID NO: 6) is also shown hybridized to the analyte via a region (detection oligonucleotide) complementary to a sequence on the analyte (first sequence) and having a suppressor complementary region. The bottom panel shows a graph that quantifies the number of spots passing intensity thresholds per FOV plotted as function of concentration.

FIG. 29 is a working example using the system described in FIG. 28 except that the target analytes (SEQ ID NO: 28) were captured on the surface of a streptavidin-coated microparticle and a non-target analyte (SEQ ID NO: 29) having a single-nucleotide substitution as compared to the target analyte was used to determine assay sensitivity. The surface-bound beads were imaged by objective-type highly inclined laminated optical sheet (HILO) microscopy (fluorescence) and bright-field microscopy (brightfield) at various concentrations of target (upper panel) versus non-target (lower panel) and also samples including both target and excess of non-target analyte (lower panel, right side). Graphical representations are also provided showing the average bead fluorescence intensity as a function of concentration of the target and non-target analyte. The bottom right graph provides mean fluorescence intensity for samples comprising mixed samples with both target and non-target analyte.

FIG. 30 is a working example using the assay described in Example 6 except that the incubation step with the second probe was performed in 25% horse serum. The graph depicts the number of bright fluorescent puncta (spots) and the relative intensity of each in samples with our without analyte (left vs right) and with or without horse serum (bottom vs top).

FIG. 31 is a working example using a detection systems similar to that described in FIG. 28 except that a biotinylated capture oligonucleotide is used to localize the analyte to a surface. The biotinylated capture oligonucleotide (SEQ ID NO: 30) is shown hybridized to analyte (SEQ ID NO: 31). Signal regions (SEQ ID NO: 32) and second probe (SEQ ID NO: 33)—via its detection oligonucleotide—are shown hybridized directly to the analyte. Suppressors (SEQ ID NO: 3) are also shown hybridized to the signal regions and the suppressor complementary region of the second probe is show in proximity with the suppressor oligonucleotides. The lower images were captured using total internal reflection fluorescence microscopy of chambers where analyte was present (left panel) and where no analyte was present (right panel).

FIG. 32 is a working example using the protein analyte detection system shown in panel A. Panel depicts a capture antibody attached to a planar surface via a biotin-streptavidin interaction (representing an analyte binding partner) in the presence of antigen (A). The second probe is shown comprising a detection antibody with affinity to antigen (A) conjugated to an oligonucleotide (SEQ ID NO: 11) carrying a suppressor complementary region. The signal region (first probe) is attached to the surface adjacent to the capture antibody and comprises the branched oligonucleotide configuration as shown in FIG. 26. The signal region (first probe) comprises a first oligonucleotide (SEQ ID NO: 24) and a series of second oligonucleotides (SEQ ID NO: 3) hybridized to the first oligonucleotide where in the second oligonucleotides include a region complementary to the first oligonucleotide and a region complementary to the suppressor oligonucleotides (SEQ ID NO: 25) which are shown here hybridized to the signal region. Panel B provides images captured using total internal reflection fluorescence microscopy of chambers where antigen was present (left panel) and where no antigen was present (right panel).

### DETAILED DESCRIPTION

The present disclosure describes particular embodiments and with reference to certain drawings, but the subject matter is not limited thereto. The drawings described are only schematic and are non-limiting. In the drawings, the size of some of the elements may be exaggerated or distorted and not drawn on scale for illustrative purposes.

The present disclosure will provide description to the accompanying drawings, in which some, but not all embodiments of the subject matter of the disclosure are shown. Indeed, the subject matter may be embodied in many different forms and should not be construed as limited to the embodiments set forth herein, rather, these embodiments are provided so that this disclosure satisfies all the legal requirements.

Certain terminology is used in the following description for convenience only and is not limiting.

### Definitions

Where the elements of the disclosure are designated as “a” or “an” in first appearance and designated as “the” or “said” for second or subsequent appearances unless something else is specifically stated. Unless specifically set forth herein, the terms “a,” “an” and “the” are not limited to one element, but instead should be read as meaning “at least one” or “one or more.” As used herein “another” means at least a second or more. The terminology includes the words noted above, derivatives thereof and words of similar import.

The use of the term “or” in the claims is used to mean “and/or” unless explicitly indicated to refer to alternatives only or the alternatives are mutually exclusive.

Use of the term “about”, when used with a numerical value, is intended to include +/−10%. For example, if a number of amino acids is identified as about 200, this would include 180 to 220 (plus or minus 10%).

Unless otherwise defined, all technical and scientific terms used herein have the same meaning as commonly understood by one of ordinary skill in the art.

As used herein “affinity to an analyte” and “affinity to the analyte” should be understood to include not only affinity to the analyte itself but can also include affinity to a component associated with an analyte. By way of example, but not limitation, an analyte could be linked to a binding partner prior to application to the systems and methods of the present disclosure (where the binding partner has affinity to the first analyte binding region of the first probe). Thus, it should be understood that when a part or a component of the system is said to have “affinity to an analyte” or “affinity to the analyte,” this can mean affinity to the binding partner to the analyte. In other words, affinity should be understood to include affinity to either the analyte itself or to a component associated with the analyte.

As used herein, the terms “peptide,” “polypeptide” and “protein” are used interchangeably and refer to amino acid sequences of any length.

As used herein “is not complementary” means a given sequence is not fully complementary to another sequence, but may hybridize under annealing conditions that are of an extended time and low temperature, but would not be expected to anneal or hybridize under standard conditions. For example, standard conditions can be a temperature of about 25° C., sodium ion concentration of about 150 mM in an aqueous solution at a pH of about 7, with a concentration of about 50 nM of the oligonucleotide sequence in excess over the other sequence.

As used herein, “complementary” means a given sequence is at least partly, if not fully, complementary to another sequence, and can hybridize under annealing conditions that are of an extended time and low temperature, and would be expected to anneal or hybridize under standard conditions. For example, standard conditions can be a temperature of about 25° C., sodium ion concentration of about 150 mM in an aqueous solution at a pH of about 7, with a concentration of about 50 nM of the oligonucleotide sequence in excess over the other sequence.

The general principle of the systems and methods of the present disclosure includes a first probe capable of stably binding to an analyte via a first analyte binding region and including a signal region that includes a plurality of suppressors. A second probe can transiently bind to the analyte (or to the first probe) when the analyte is bound to the first probe and can directly or indirectly remove or degrade one or more of the suppressors. In certain aspects, the signal region can further include label probes whose signal is prevented by the bound suppressors, where removal or degradation of the suppressors can allow detection of the label probes. By way of example, but not limitation, the removal or degradation of the suppressors can allow detection of the label probes by dequenching a fluorophore. In certain other aspects, removal or degradation of the suppressors can permit binding of label probes to the signal region allowing for detection of the label probes bound to the first probe. Even in this situation, the suppressors can be labeled with a quencher to suppress fluorescence from labels which bind to the same first probe, so that removal of all (or nearly all) of the suppressors is required for a maximally bright signal from the fluorescent labels that bind to the first probe. In other words, the suppressor removal could both remove one or more quenchers and permit the subsequent binding of one or more label probes, both of which would contribute to the accumulation of fluorescent signal. The system and methods thus achieve signal amplification through repeated binding of the second probes to the analyte or to first probe(s) bound to the analyte. This amplification is not necessarily enzymatic, but can optionally incorporate enzymes for specific functions such as, by way of example, but not limitation, degrading or removing the suppressors or generating signal in conjunction with or as part of the label probe.

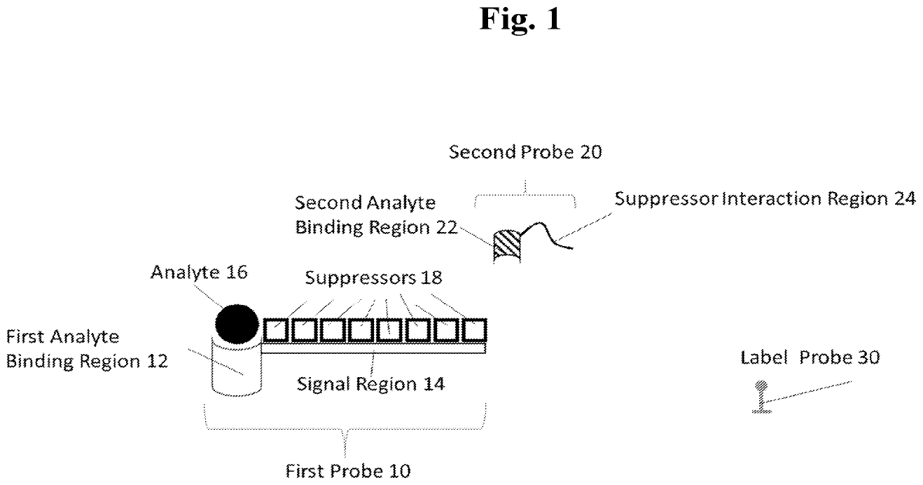

As shown in FIG. 1, an exemplary system of the present disclosure can include a first probe 10 that includes a first analyte binding region 12 that can stably bind with at least one analyte 16 and a signal region 14 which is associated with a plurality of suppressors 18. A second probe 20 that includes a second analyte binding region 22 that can bind transiently to the analyte 16 and a suppressor interacting region 24 that can directly or indirectly remove or degrade one or more suppressors 18. The label probe 30 can be capable of generating a signal and can either associate with the signal region 14 after the removal or degradation of one or more suppressors 18 or can be present in the signal region where the suppressors 18 prevent signal from the label probe 30 until they are removed or degraded. Even if some suppressors 18 have been removed, the remaining suppressors 18 may prevent or reduce signal from any label probes 30 associated with the first probe.

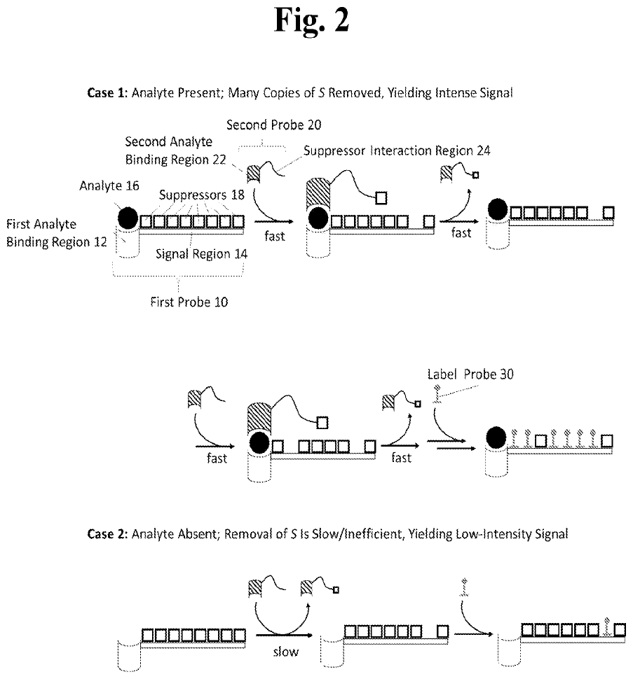

Case 1 of FIG. 2 further depicts an exemplary system and method of the present disclosure where the first probe 10 includes a first analyte binding region 12 for binding at least one analyte 16 and a signal region 14 which is associated with a plurality of suppressors 18. A second probe 20 that includes a second analyte binding region 22 and a suppressor interacting region 24 can quickly, relative to the dissociation of the analyte 16 from the first analyte binding region 12, bind to the analyte via the second analyte binding region 22 and directly or indirectly remove or degrade one or more of the suppressors 18. This binding can increase the local concentration of second probe 20 significantly and increase the rate of suppressor removal or degradation by the second probe bound to the analyte associated with the first probe relative to the rate of suppressor removal or degradation by a second probe not bound to an analyte associated with the first probe. By having a more transient interaction between the second analyte binding region and the analyte than between the first analyte binding region and the analyte, multiple copies of the suppressors can be removed quickly by the successive binding of multiple second probes to the analyte associated with the first probe which can result in a more intense signal than in the absence of analyte as discussed below. By removing the one or more suppressors 18, binding sites are exposed for the label probe 30 which can then bind where the suppressors were removed and can then be used for signal detection. As shown in Case 2 of FIG. 2, in the absence of analyte, the removal of suppressors 18 by the second probe 20 is slow because the local concentration of the second probe 20 is not enhanced by its binding to the analyte 16 via the second analyte binding region 22, which results in a slow and inefficient reaction that yields a low intensity signal. While each second probe may remove or degrade one or more suppressors, to ensure the best discrimination between repeated second probe binding to the analyte and inefficient binding of the second probe to the first probe not bound to an analyte, each second probe should only remove a minority of the suppressors present on the first probe, so that multiple successive binding events of different copies of the second probe are required for a maximally intense signal.

Detection of the analyte can therefore be achieved by determining whether a first probe has had a certain minimum number of suppressors removed. Furthermore, counting or otherwise quantifying the number of copies of the first probe that have had a certain minimum of suppressors removed can provide a mechanism for quantifying the analyte, since the number of copies of the first probe associated with the analyte is expected to be proportional to the concentration of the analyte, assuming that the first probe is present in excess relative to the analyte.

While both Case 1 and Case 2 of FIG. 2 will result in a distribution (e.g., a Poisson or binomial distribution, or other distribution) with respect to the number of suppressors removed from each first probe, the average number of suppressors removed from the first probes will be distinct in Case 1 and Case 2. For example, if Case 1 results in the removal or degradation of an average of 20 copies of the suppressors from each first probe, but Case 2 results in the removal or degradation of an average of only 2 copies of the suppressors from each first probe, and both cases exhibit Poisson statistics with respect to the number of copies of the suppressors that have been removed or degraded, the removal of at least 20 copies of suppressors from a copy of the first probe is predicted to happen with only 6.4×10⁻¹²% probability in the absence of the analyte, but with >50% probability in the presence of analyte. The detection of a copy of the first probe from which 20 copies of the suppressors have been removed is therefore unlikely to have arisen from Case 2 (absence of analyte) and very likely to have arisen from Case 1 (presence of analyte).

Thus, the general principles of the present disclosure can be described as a system which includes a first probe that includes a first analyte binding region and a signal region that has multiple suppressors, where the first analyte binding region can associate with an analyte more stably than a second probe which can transiently associate with the analyte (or the first probe when bound to the analyte), where the second probe can bind to the analyte (or the first probe when bound to the analyte), directly or indirectly remove or degrade one or more of the suppressors after a sufficient length of time, followed by detection of the removal of the suppressors by a suitable detection method such as, by way of example but not limitation, allowing a signal from a signal region of the first probe to become distinguishable after removal of the one or more suppressors such as removing a quencher (suppressor) from the signal region of the first probe to allow a fluorophore signal associated with the signal region to be detected, adding a label that can bind to where the one or more suppressors have been removed or other suitable method for detecting the removal of the one or more suppressors, or detecting a change in the properties of the first probe such as a change in net charge, mass, charge/mass ratio, or hydrodynamic radius as measured by, for example, a mobility shift detectable by gel electrophoresis or column chromatography (e.g., ion-exchange, size exclusion, gel-filtration, reverse-phase, HPLC, or FPLC) or by dynamic light scattering or any other technique that can detect a change in one or more of the above properties. For example, if each suppressor were linked to a moiety with large molecular weight (e.g., a polyethylene glycol or other polymer with molecular weight>1,000 Daltons) or large hydrodynamic radius (e.g., a rigid double-stranded DNA>10 nanometers in length) or a charge/mass ratio significantly different from the rest of the first probe (e.g., a polymer, such as a polyhistidine, that bears a net positive charge at the same pH at which the complex bears an overall negative or neutral charge), the removal of each suppressor would reduce the overall mass and/or hydrodynamic radius of the first probe complex, or significantly alter its net charge or charge/mass ratio; this change would result in a change in electrophoretic mobility of the first probe upon removal of suppressors, which could be detected by agarose gel electrophoresis or polyacrylamide gel electrophoresis. By way of example, but not limitation, first probes bound to about 20 copies of a label probe linked to a polyethylene glycol spacer with a molecular weight of about 2,000 Daltons—i.e., first probes which were bound to an analyte during previous incubation steps-will have an overall molar mass about 36,000 Daltons greater than first probes bound to about 2 copies of the same label probe—i.e., first probes which were not bound to an analyte during previous incubation steps. Such a difference in molecular weight could be detected by a difference in mobility of the first probe in a 5-10% polyacrylamide gel run at about 10 V/cm for 20-30 minutes in a suitable running buffer such as tris-borate-EDTA. In such an experiment, the presence of the shifted band bearing multiple copies of the polyethylene glycol spacer (and, hence, the presence of the analyte) could be sensitively detected, for example, by the use of label probes bearing a fluorophore or a radioactive isotope such as phosphorus-32 or sulfur-35 and using an appropriate fluorometric, radiometric, or phosphorometric imager or scanner to detect the shifted band relative to a control band representing the first probe which has been incubated under identical or nearly identical conditions but in the absence of the analyte. In general, the mobility of the first probes will also be different than that of individual excess label probes which may be present in the mixture, but will be of much lower molecular weight than first probes bound to multiple label probes. Alternatively, the removal of one or more suppressors could expose one or more binding sites for a label probe that is linked to a moiety with large molecular weight, large hydrodynamic radius, or a charge/mass ratio significantly different from the first probe; the binding of multiple such label probes to a common first probe would result in a significant increase in mass and/or hydrodynamic radius of the first probe complex, or significantly alter the net charge or charge/mass ratio of the first probe complex, resulting in a change in electrophoretic mobility of the first probe upon binding of the label.

General design considerations for the systems and methods of the present disclosure can be the requirement that removal or degradation of multiple copies of the suppressors from the signal region be more efficient in the presence of analyte than its absence. To achieve this difference in efficiency, the follow conditions can be satisfied: (1) the interaction between the second probe and the analyte (or the second probe and the first probe when it is bound to the analyte) should have rate constants of binding and dissociation sufficiently fast that multiple copies of the second probe can bind to and dissociate from each copy of the analyte (or the first probe bound to the analyte) within the time frame of the experiment; (2) when the second probe is bound to the analyte (or to the first probe when it is bound to the analyte), the removal of one or more suppressors from the signal region should be much more efficient than when the second probe is not bound to the analyte (or to the first probe), for example, the association of the second probe with the analyte (or the first probe) may produce a local effective concentration of second probe in the proximity of the associated signal region that is >10, >100, or >1000 times higher than the bulk concentration of the second probe which can accelerate the removal of the suppressors from the signal region; (3) the binding of the second probe to the analyte (or the first probe when it is bound to the analyte) should occur with sufficiently high affinity that there is enough time for the second probe to remove or degrade one or more suppressors with high probability before dissociating; and (4) the rate constant of dissociation of the analyte from the first analyte binding region should be significantly slower than that of dissociation of the second probe from the analyte (or the first probe when it is bound to the analyte), to allow for multiple sequential copies of the second probe to bind to the same complex of the analyte and first probe before the analyte dissociates from the first analyte binding region.

### Systems

In some embodiments an analyte detection system is provided that can include: a plurality of first probes, each first probe comprising a first analyte binding region and a signal region, wherein the first analyte binding region has affinity to an analyte, wherein the signal region is associated with a plurality of suppressors and a plurality of label probes, wherein the suppressors prevent detection of the label probes when both are associated with the signal region; and a plurality of second probes, each second probe comprising a second analyte binding region and a suppressor interacting region, wherein the second analyte binding region has affinity to the analyte, wherein the suppressor interacting region is indirectly or directly capable of binding to, removing, or degrading one or more of the suppressors; wherein the second analyte binding region of the second probe is able to bind the analyte while the analyte is bound to the first analyte binding region of the first probe, wherein the interaction of the first analyte binding region to the analyte possess a rate constant of dissociation that is slower than the rate constant of dissociation between the second analyte binding region and the analyte, wherein when one of the second probes binds to the analyte bound to one of the first probes, one or more of the suppressors may be removed from the first probe or degraded, and wherein upon removal or degradation of a sufficient number of suppressors, the signal arising from the plurality of label probes becomes distinguishable from the signal arising from the first probe in the absence of the analyte.

In some embodiments, an analyte detection system is provided that can include: a plurality of first probes, each first probe comprising a first analyte binding region and a signal region, wherein the first analyte binding region has affinity to an analyte, wherein the signal region is associated with a plurality of suppressors; a plurality of second probes, each second probe comprising a second analyte binding region and a suppressor interacting region, wherein the second analyte binding region has affinity to the analyte, wherein the suppressor interacting region is indirectly or directly capable of binding to, removing or degrading one or more of the suppressors, wherein the second analyte binding region of the second probe is able to bind the analyte while the analyte is bound to the first analyte binding region of the first probe, wherein the interaction of the first analyte binding region to the analyte possesses a rate constant of dissociation that is slower than the rate constant of dissociation between the second analyte binding region and the analyte, wherein when one of the second probes binds to the analyte while bound to one of the first probes, one or more of the suppressors may be removed from the first probe or degraded; and a plurality of label probes, each label probe having an affinity to the signal region of the first probe, wherein upon removal or degradation of one or more suppressors, one or more label probes are able to bind to the signal region of the first probe.

In any of the above embodiments, it should be understood that the analyte binding region and the signal region of the first probe may be distinct molecular entities that are indirectly associated with each other via their proximity on a solid support such as a planar surface, a microparticle, or a nanoparticle. For example, the analyte binding region may comprise a biotinylated capture oligonucleotide that is bound to a streptavidin-coated surface and the signal region may comprise a separate biotinylated oligonucleotide that comprises a plurality of suppressor binding sequences that are complementary to sequences on suppressor oligonucleotides. In this example, the binding sites for the capture oligonucleotide and the signal oligonucleotide should be within 100 nanometers of one another, and more preferably, within 20 nanometers of one another so that when the second probe associates with the analyte bound the capture oligonucleotide, it is in close enough proximity to interact with the suppressors bound the signal regions of the first probe.

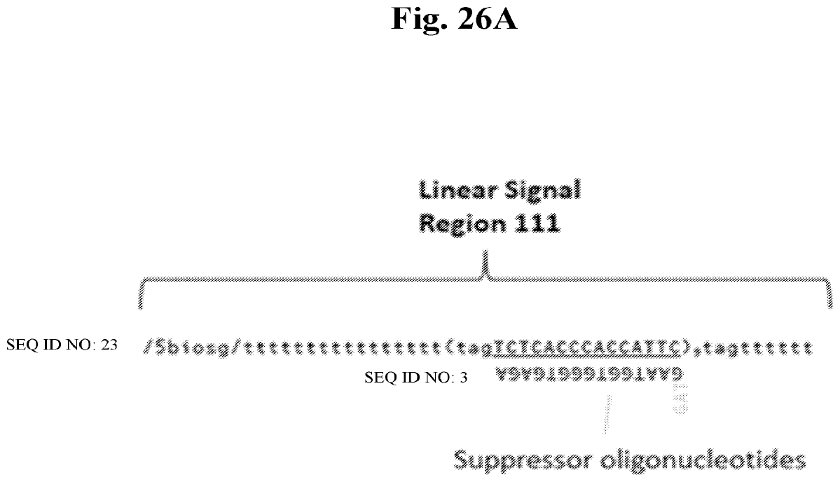

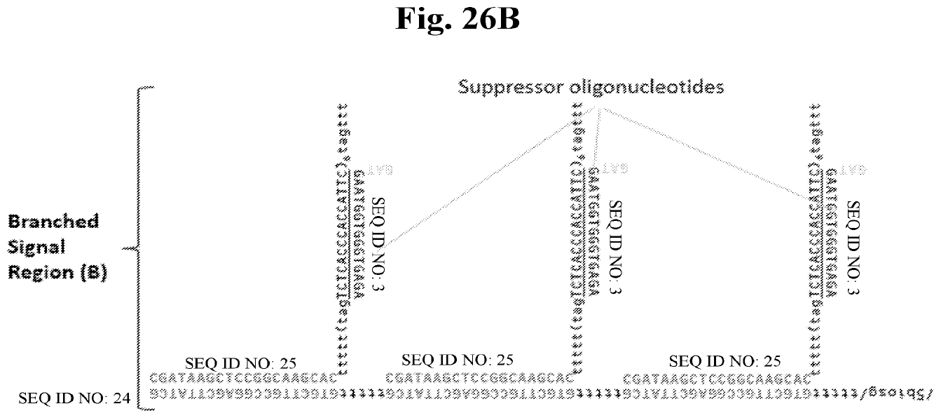

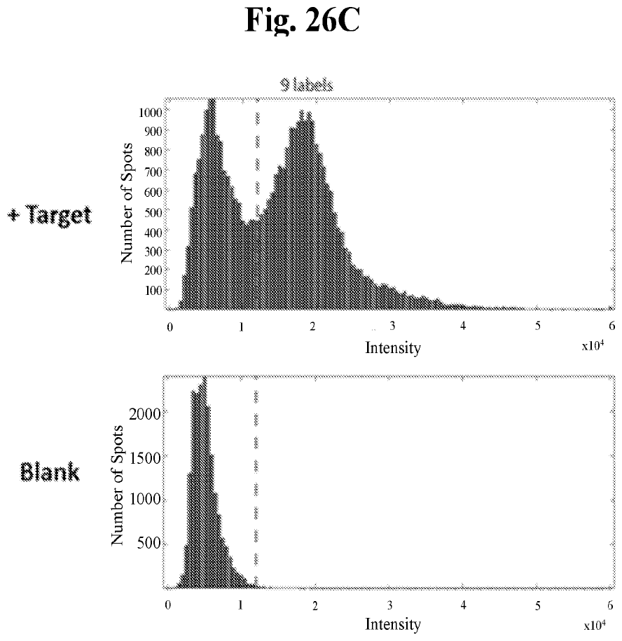

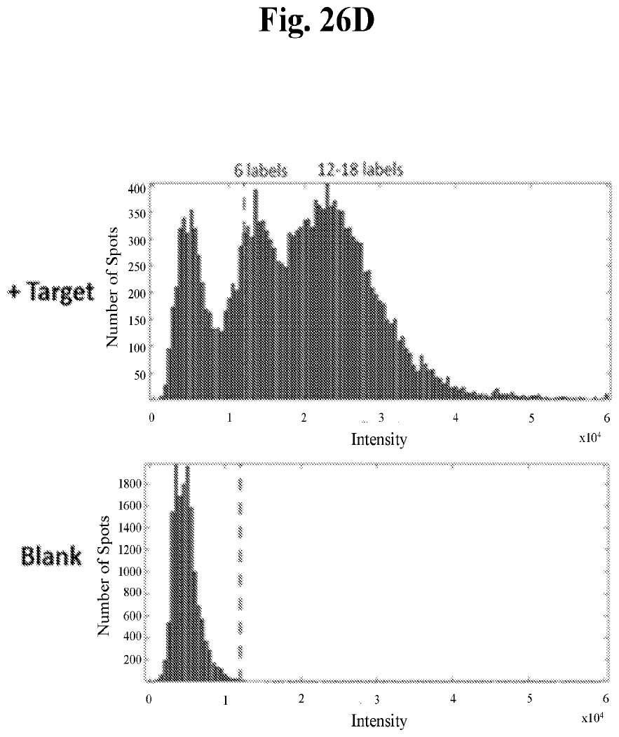

In some embodiments, a branched signal region structure can be used. Referring to the above example, the separate biotinylated oligonucleotide may instead include a plurality of sequences that are complementary to a sequence this is present in a plurality of third oligonucleotide. In this example, the third oligonucleotides comprise a first sequence complementary to the separate biotinylated oligonucleotide and a second sequence that represents the suppressor binding region (in the instance the suppressors are oligonucleotides) wherein the second sequence is not complementary to the separate biotinylated oligonucleotide. In this way, there are multiple third oligonucleotides associated with the separate biotinylated oligonucleotide as shown in panel B of FIG. 26.

Embodiments of the technology relate to systems for detecting analytes. For example, in some embodiments, the technology provides a system for quantifying one or more target analytes as described herein, wherein the system comprises a first probe comprising an analyte binding region, signaling region, suppressors, and optional label probes as described herein and a second probe comprising a suppressor interacting region as described herein. Furthermore, some system embodiments comprise a detection component that records a signal from the first probe after incubation of the first probe and the second probe with a sample comprising an analyte, if present. For example, in some embodiments the detection component records a signal produced from the first probe, e.g., after interaction of the first probe and the second probe with an analyte. In some embodiments, the detection component records the intensity of a signal provided by a first probe comprising a plurality of label probes.

System embodiments can comprise analytical processes (e.g., embodied in a set of instructions, e.g., encoded in software, that direct a microprocessor to perform the analytical processes) to process a signal (e.g., from a first probe comprising a plurality of label probes) and to identify a sample as a sample comprising an analyte. In some embodiments, analytical processes use the intensity of the signal produced by a first probe comprising a plurality of label probes as input data. In some embodiments, analytical processes such as, by way of example but not limitation, normalization or flattening algorithms, correct for systematic variations in signal levels caused by known biases in the measurement apparatus such as, by way of example but not limitation, variations in the intensity of fluorescence excitation over a microscopic field of view. In some embodiments, systems comprise an analyte. Embodiments of systems are not limited in the analyte that is detected. For example, in some embodiments the analyte is polypeptide, e.g., a protein or a peptide. In some embodiments, the target analyte is a nucleic acid. In some embodiments, the target analyte is a small molecule, metabolite, metal ion, biomolecule, or other molecule or entity as described herein.

Some system embodiments of the technology comprise components for the detection and quantification of an analyte. Some system embodiments comprise a detection component that is a fluorescence microscope comprising an illumination configuration to excite the signaling component of label probes associated with the signal region of first probes. Some embodiments comprise a fluorescence detector, e.g., a detector comprising an intensified charge coupled device (ICCD), an electron-multiplying charge coupled device (EM-CCD), a complementary metal-oxide-semiconductor (CMOS), a photomultiplier tube (PMT), an avalanche photodiode (APD), and/or another detector capable of detecting fluorescence emission from single chromophores. Some particular embodiments comprise a component configured for lens-free imaging, e.g., a lens-free microscope, e.g., a detection and/or imaging component for directly imaging on a detector (e.g., a CMOS) without using a lens.

Some embodiments comprise a computer and software encoding instructions for the computer to perform, e.g., to control data acquisition and/or analytical processes for processing data.

Some embodiments comprise optics, such as lenses, mirrors, dichroic mirrors, optical filters, etc., e.g., to detect fluorescence selectively within a specific range of wavelengths or multiple ranges of wavelengths.

For example, in some embodiments, computer-based analysis software is used to translate the raw data generated by the detection assay (e.g., the presence, absence, or amount of one or more analytes) into data of predictive value for a clinician. The clinician can access the predictive data using any suitable means.

Some system embodiments comprise a computer system upon which embodiments of the present technology may be implemented. In various embodiments, a computer system includes a bus or other communication mechanism for communicating information and a processor coupled with the bus for processing information. In various embodiments, the computer system includes a memory, which can be a random access memory (RAM) or other dynamic storage device, coupled to the bus, and instructions to be executed by the processor. Memory also can be used for storing temporary variables or other intermediate information during execution of instructions to be executed by the processor. In various embodiments, the computer system can further include a read only memory (ROM) or other static storage device coupled to the bus for storing static information and instructions for the processor. A storage device, such as a magnetic disk or optical disk, can be provided and coupled to the bus for storing information and instructions.

In various embodiments, the computer system is coupled via the bus to a display, such as a cathode ray tube (CRT) or a liquid crystal display (LCD), for displaying information to a computer user. An input device, including alphanumeric and other keys, can be coupled to the bus for communicating information and command selections to the processor. Another type of user input device is a cursor control, such as a mouse, a trackball, or cursor direction keys for communicating direction information and command selections to the processor and for controlling cursor movement on the display. This input device typically has two degrees of freedom in two axes, a first axis (e.g., x) and a second axis (e.g., y), that allows the device to specify positions in a plane.

A computer system can perform embodiments of the present technology. Consistent with certain implementations of the present technology, results can be provided by the computer system in response to the processor executing one or more sequences of one or more instructions contained in the memory. Such instructions can be read into the memory from another computer-readable medium, such as a storage device. Execution of the sequences of instructions contained in the memory can cause the processor to perform the methods described herein. Alternatively, hard-wired circuitry can be used in place of or in combination with software instructions to implement the present teachings. Thus, implementations of the present technology are not limited to any specific combination of hardware circuitry and software.

The term “computer-readable medium” as used herein refers to any medium that participates in providing instructions to the processor for execution. Such a medium can take many forms, including but not limited to, non-volatile media, volatile media, and transmission media. Examples of non-volatile media can include, but are not limited to, optical or magnetic disks, such as a storage device. Examples of volatile media can include, but are not limited to, dynamic memory. Examples of transmission media can include, but are not limited to, coaxial cables, copper wire, and fiber optics, including the wires that comprise the bus.

Common forms of computer-readable media include, for example, a floppy disk, a flexible disk, hard disk, flash medium, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

Various forms of computer readable media can be involved in carrying one or more sequences of one or more instructions to the processor for execution. For example, the instructions can initially be carried on the magnetic disk of a remote computer. The remote computer can load the instructions into its dynamic memory and send the instructions over a network connection (e.g., a LAN, a WAN, the internet, a telephone line). A local computer system can receive the data and transmit it to the bus. The bus can carry the data to the memory, from which the processor retrieves and executes the instructions. The instructions received by the memory may optionally be stored on a storage device either before or after execution by the processor.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

In accordance with such a computer system, some embodiments of the technology provided herein further comprise functionalities for collecting, storing, and/or analyzing data (e.g., presence, absence, concentration of an analyte). For example, some embodiments contemplate a system that comprises a processor, a memory, and/or a database for, e.g., storing and executing instructions, analyzing label signals, signal intensities, and/or detection data, performing calculations using the data, transforming the data, and storing the data. In some embodiments, an algorithm applies a statistical model to the data.

Many diagnostics involve determining the presence of, or a nucleotide sequence of, one or more nucleic acids.

In some embodiments, an equation comprising variables representing the presence, absence, concentration, amount, or sequence properties of one or more analytes produces a value that finds use in making a diagnosis or assessing the presence or qualities of an analyte. As such, in some embodiments this value is presented by a device, e.g., by an indicator related to the result (e.g., an LED, an icon on a display, a sound, or the like). In some embodiments, a device stores the value, transmits the value, or uses the value for additional calculations. In some embodiments, an equation comprises variables representing the presence, absence, concentration, amount, or properties of one or more analytes.

Thus, in some embodiments, the present technology provides the further benefit that a clinician, who is not likely to be trained in analytical assays, need not understand the raw data. The data are presented directly to the clinician in its most useful form. In some embodiments, the clinician is then able to utilize the information to optimize the care of a subject. The present technology contemplates any method capable of receiving, processing, and transmitting the information to and from laboratories conducting the assays, information providers, medical personal, and/or subjects. For example, in some embodiments of the present technology, a sample is obtained from a subject and submitted to a profiling service (e.g., a clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center or subjects may collect the sample themselves and directly send it to a profiling center. Where the sample comprises previously determined biological information, the information may be directly sent to the profiling service by the subject (e.g., an information card containing the information may be scanned by a computer and the data transmitted to a computer of the profiling center using electronic communication systems). Once received by the profiling service, the sample is processed, and a profile is produced that is specific for the diagnostic or prognostic information desired for the subject. The profile data are then prepared in a format suitable for interpretation by a treating clinician. For example, rather than providing raw data, the prepared format may represent a diagnosis or risk assessment for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor. In some embodiments, the information is first analyzed at the point of care or at a regional facility. The raw data are then sent to a central processing facility for further analysis and/or to convert the raw data to information useful for a clinician or patient. The central processing facility provides the advantage of privacy (all data are stored in a central facility with uniform security protocols), speed, and uniformity of data analysis. The central processing facility can then control the fate of the data following treatment of the subject. For example, using an electronic communication system, the central facility can provide data to the clinician, the subject, or researchers. In some embodiments, the subject is able to access the data using the electronic communication system. The subject may choose further intervention or counseling based on the results. In some embodiments, the data are used for research use. For example, the data may be used to further optimize the inclusion or elimination of markers as useful indicators of a particular condition associated with the disease.

### Analyte

In any of the foregoing embodiments, the analyte can be any suitable molecule for detection using the systems and methods of the present disclosure. By way of example, but not limitation, the analyte can be a DNA oligonucleotide, a biologically derived DNA sequence or DNA fragment, an RNA oligonucleotide, a biologically derived RNA sequence or RNA fragment, a protein, a polypeptide, an oligopeptide, a carbohydrate such as a polysaccharide or oligosaccharide, a small molecule or metabolite, or a combination thereof. By way of further example, but not limitation, the analyte can include a ribonucleoprotein, ribosome, viral particle, DNA-binding proteins bound to a DNA sequence, proteins and lipids (lipoproteins), or proteins and carbohydrates (proteoglycans, glycoproteins). In any of the foregoing embodiments, where the analyte is a nucleic acid, the analyte may comprise a sequence variant such as, by way of example but not limitation, a single-nucleotide polymorphism, a single-nucleotide mutation, an insertion mutation comprising one or more nucleotides, a deletion mutation comprising one or more nucleotides, a substitution mutation comprising one or more nucleotides, an abasic site, a single-stranded or double-stranded break, or one or more nucleotides bearing chemical modifications such as 5-methylcytosine, N⁶-methyladenosine, or other chemical modifications. In any of the foregoing embodiments, where the analyte is a nucleic acid or includes a nucleic acid, such as, by way of example but not limitation, a DNA oligonucleotide, a biologically derived DNA sequence or DNA fragment, an RNA oligonucleotide, a biologically derived RNA sequence or RNA fragment, the analyte can have any suitable length. By way of example, but not limitation, in such embodiments, the nucleic acid component of the analyte can have a length of about 1 to about 500 nucleotides, about 1 to about 400 nucleotides, about 1 to about 300 nucleotides, about 1 to about 300 nucleotides, about 1 to about 200 nucleotides, about 1 to about 100 nucleotides, about 1 to about 50 nucleotides, about 1 to about 40 nucleotides, about 1 to about 30 nucleotides, about 1 to about 25 nucleotides, about 1 to about 20 nucleotides, about 1 to about 10 nucleotides, about 5 to about 500 nucleotides, about 5 to about 400 nucleotides, about 5 to about 300 nucleotides, about 5 to about 200 nucleotides, about 5 to about 100 nucleotides, about 5 to about 50 nucleotides, about 5 to about 40 nucleotides, about 5 to about 30 nucleotides, about 5 to about 25 nucleotides, about 5 to about 20 nucleotides, about 5 to about 10 nucleotides, about 10 to about 500 nucleotides, about 10 to about 400 nucleotides, about 10 to about 300 nucleotides, about 10 to about 200 nucleotides, about 10 to about 100 nucleotides, about 10 to about 50 nucleotides, about 10 to about 40 nucleotides, about 10 to about 30 nucleotides, about 10 to about 25 nucleotides, about 10 to about 20 nucleotides, about 20 to about 500 nucleotides, about 20 to about 400 nucleotides, about 20 to about 300 nucleotides, about 20 to about 200 nucleotides, about 20 to about 100 nucleotides, about 20 to about 50 nucleotides, about 20 to about 40 nucleotides, about 20 to about 30 nucleotides, about 20 to about 25 nucleotides, about 25 to about 500 nucleotides, about 25 to about 400 nucleotides, about 25 to about 300 nucleotides, about 25 to about 200 nucleotides, about 25 to about 100 nucleotides, about 25 to about 50 nucleotides, about 25 to about 40 nucleotides, about 25 to about 30 nucleotides, about 30 to about 500 nucleotides, about 30 to about 400 nucleotides, about 30 to about 300 nucleotides, about 30 to about 200 nucleotides, about 30 to about 100 nucleotides, about 30 to about 50 nucleotides, about 30 to about 40 nucleotides, about 40 to about 500 nucleotides, about 40 to about 400 nucleotides, about 40 to about 300 nucleotides, about 40 to about 200 nucleotides, about 40 to about 100 nucleotides, about 40 to about 50 nucleotides, about 50 to about 500 nucleotides, about 50 to about 400 nucleotides, about 50 to about 300 nucleotides, about 50 to about 200 nucleotides, about 50 to about 100 nucleotides, about 100 to about 500 nucleotides, about 100 to about 400 nucleotides, about 100 to about 300 nucleotides, about 100 to about 200 nucleotides, about 200 to about 500 nucleotides, about 200 to about 400 nucleotides, about 200 to about 300 nucleotides, about 300 to about 500 nucleotides, about 300 to about 400 nucleotides, about 400 to about 500 nucleotides, about 1, 5, 10, 20, 25, 30, 40, 50, 100, 200, 300, 400, 500 or more nucleotides. By way of further example, but not limitation, the analyte can be a messenger RNA, pre-messenger RNA, ribosomal RNA, transfer RNA, non-coding RNA, small nucleolar RNA, small nuclear RNA, extracellular RNA, microRNA, primary microRNA, pre-microRNA, long non-coding RNA, long intervening non-coding RNA, circular RNA, piwi-interacting RNA, trans-renal RNA, viral RNA, cell-free DNA, mitochondrial DNA, extracellular DNA, circulating tumor DNA, trans-renal DNA, or viral DNA.

In any of the embodiments disclosed herein, the analyte may be present in the sample at concentrations as low as 1 femtomolar. In embodiments where the analyte is a nucleic acid, the analyte may be present in a mixed sample at concentrations as low as 1 femtomolar where the sample includes non-target analytes at a concentration in excess of 1 femtomolar and in some embodiments, in excess of 1 picogram per milliliter, wherein the analyte and non-target analyte only differ by a single nucleotide substitution. In this embodiment, the present system, methods, and kits can be used to selectively determine the presence of the analyte in such sample.

It should be understood that the analyte can be coupled to another component to which the first analyte binding region and/or the second analyte binding region can bind. By way of example, but not limitation, the analyte can be conjugated to biotin for coupling with streptavidin. Alternatively, by way of example, but not limitation, where the analyte comprises an oligonucleotide, a capture oligonucleotide as disclosed herein can bind directly to the analyte. Alternatively, by way of example, but not limitation, where the analyte comprises is a peptide, carbohydrate, epigenetic modification, or post-translational modification, a capture antibody as disclosed herein can bind directly to the analyte. It should be understood that while some of the examples demonstrated an analyte that was biotinylated or otherwise modified to allow interaction with a surface, this is not required by the instant invention and as discussed above, in the instance the analyte is a nucleic acid, a capture oligonucleotide that is associated with or capable of association with a microparticle, nanoparticle, or planar surface may be used.

It should be understood that the sample containing the analyte may comprise many non-target analytes at concentrations well in excess of the target analyte. It should also be understood that the system, methods and kits of the present disclosure are able to be used to detect analytes in these mixed samples, including biological samples such as saliva, blood, urine, semen, cell extract, and other bodily fluids.

It should be understood that throughout this disclosure, various chemistries can be used to couple elements of the systems and methods of the disclosures. By way of example, but not limitation, these can include streptavidin-biotin interaction, gold-thiol interactions, click chemistry reagent pairs, such as azide+alkyne and trans-cyclooctene+tetrazine, EDC-NHS coupling between carboxylic acids and amines, thiol-maleimide coupling, amine-NHS ester coupling, electrostatic interactions, coaxial stacking of nucleic acid duplexes, hydrogen bonding interactions, hydrophobic interactions, van der Waals forces, and others. Such chemistries and standard coupling techniques can be applied to, by way of example but not limitation, coupling of first analyte binding regions, signal regions, first probes, second probes, labels and label probes, nanoparticles and solid supports.

### First Probes

The systems, kits and methods of the present disclosure can utilize first probes as described herein. In any of the embodiments of the present disclosure, the first probes or plurality thereof, can include a first analyte binding region and a signal region. It should be understood that a first probe can include one or more signal regions and/or one or more first analyte binding regions. In one example, each first probe comprises a single first analyte binding region and a plurality of signal regions. By way of example, but not limitation, the first probe can include 1, 2, 3, 4, 5, 6, 7, 8, 9, 10, 11, 12, 13, 14, 15, 16, 17, 18, 19, 20, or any range there between, signal regions and/or first analyte binding regions. By way of further example, but not limitation, the first probe can include 10 or more signal regions and/or first analyte binding regions.

In any of the embodiments of the present disclosure, the first probe can include a colloidal particle, a nanoparticle such as a gold nanoparticle, a lipid vesicle, a surface, a region of a surface, a phase of an aqueous two-phase system, one or more DNA oligonucleotides, one or more RNA oligonucleotides, one or more locked nucleic acid (LNA) oligonucleotides, one or more peptide nucleic acid (PNA) oligonucleotides, one or more antibodies or antibody fragments, one or more aptamers, one or more fluorophores or other chromophores, one or more reactive chemical functional groups, or combinations thereof. By way of example, but not limitation, the gold nanoparticle can have a diameter of 10-40 nm. By way of further example, but not limitation, the fluorophores or chromophores can be Cy3, Cy5, Cy5.5, Cy7, ATTO 647N, DyLight 550, DyLight 650, Alexa Fluor 488, Alexa Fluor 555, or Alexa Fluor 647. By way of still further example, but not limitation, the one or more reactive chemical functional groups can include NHS esters, amines, alkynes, azides, trans-cyclooctenes, or tetrazines. It should be understood that the foregoing components can be or be a part of the first analyte binding region, the signal region, both, or a label probe associated with the first probe.

In any of the embodiments of the present disclosure, the first probes, or plurality thereof, can be bound to a solid support. In certain aspects, the solid support is a nanoparticle or a microparticle.

In any of the embodiments of the present disclosure, the first probes, or plurality thereof, can be present in a solution or colloidal suspension.

### Nanoparticles, Microparticles, or Planar Surfaces

In any of the embodiments of the present disclosure, the first analyte binding region, the signal region or both of each first probe can be bound to a nanoparticle, a microparticle, or a planar surface (collectively “supports”). In such embodiments where both the first analyte binding region and the signal region are bound to the support, the first analyte binding region and the signal region of each first probe can be bound to adjacent sites on the support. By way of example, but not limitation, the first analyte binding region and the signal region can be within about 100 nm of each other. By way of further example, but not limitation, the first analyte binding region and the signal region can be within about 100 nm, about 90 nm, about 80 nm, about 70 nm, about 60 nm, about 50 nm, about 40 nm, about 30 nm, about 25 nm, about 20 nm, about 10 nm or less of each other on the nanoparticle. It should be understood that the distance between the first analyte binding region and the signal region should be small enough that the second probe can simultaneously interact with the analyte (or the first probe) and the signal region. It should be understood that, as used throughout the present disclosure, adjacent can have the foregoing meaning and ranges. By way of example, but not limitation, the support can be a nanoparticle, a gold nanoparticle, a silver nanoparticle, a metal oxide nanoparticle, a magnetic nanoparticle, a silica nanoparticle, a liposome, a lipid nanodisc, a polymer-based nanoparticle such as a polystyrene nanoparticle, a microparticle such as a bead, wherein the microparticle has a diameter of from about 1 micrometer to about 10 micrometers and more preferably, from about 5-10 micrometers. Each type of support can be readily synthesized with different surface groups for covalent or non-covalent immobilization of desired components, such as the probes of the present disclosure. By way of still further example, but not limitation, the support can be a DNA or RNA nanostructure such as DNA origami, DNA nanocubes, DNA barrels, DNA tiles, DNA bricks, RNA origami or other similar nanoscale structures formed by the self-assembly of DNA or RNA strands into intricate two- or three-dimensional shapes in a manner guided by their designed sequences. It is expected, without being bound to theory, that such supports may be particularly advantageous in the systems and methods of the present disclosure by permitting site-specific anchoring of different components to the structure, permitting one to control the relative positions of multiple functional components.

By way of still further example, but not limitation, the signal region of the first probe can be biotinylated such that the signal region is bound to the support through a streptavidin-biotin interaction.

By way of still further example, but not limitation, the first analyte binding region can be in the form of a capture oligonucleotide. In this example, the capture oligonucleotide is biotinylated such that the capture oligonucleotide is bound to the support through the streptavidin-biotin interaction and wherein the signal region of the first probe can be bound to adjacent sites on a support.

It should be understood that the first probe can be further bound to a solid support via the nanoparticle or directly to a solid support. By way of example, but not limitation, such linkage can be via a streptavidin-biotin interaction.

### First Analyte Binding Region

In any of the foregoing embodiments of the present disclosure, the first probe can include a first analyte binding region and a signal region. In some instances, the first analyte binding region can be separate from the first probe. For example, the first analyte binding region may be a capture oligonucleotide associated with a support and the signal region may be a separate oligonucleotide attached to the support at close proximity as discussed above. In another example, the capture oligonucleotide may comprise a first sequence that is complementary to the analyte (when the analyte is a nucleic acid) and a second sequence (or plurality of second sequences) that has sequences complementary to a sequence on a separate oligonucleotide, wherein the separate oligonucleotide comprise the signal region.

Preferably, the interaction between the second probe and the analyte should have rate constants of binding and dissociation that are sufficiently fast for multiple copies of the second probe to bind to, and dissociate from, each copy of the analyte during the timeframe of an experiment.

The first analyte binding region can have affinity to an analyte. The interaction between the first analyte binding region the analyte can be characterized by a rate constant of dissociation which refers to a proportionality constant k_{dissoc} that relates the instantaneous rate of change in concentration of a molecular complex with respect to time, dC/dt, to the current concentration C of the complex, according to the equation:

$$\frac{d C}{d t} = - {k}_{d i s s o c} C$$

and which for homogeneous first-order chemical reactions is equal to the reciprocal of the average dwell time of an individual complex prior to dissociating into two or more constituents. This rate constant can be measured using several established approaches, including biolayer interferometry, surface plasmon resonance, and single-molecule total internal reflection fluorescence (TIRF) microscopy. In TIRF microscopy, by way of example, but not limitation, one can first immobilize the first analyte binding region to a suitable solid support, such as a coverslip, via a suitable immobilization chemistry, such as the biotin-streptavidin interaction. Next, the analyte, labeled with a suitable fluorophore such as Cy5, can be introduced into an aqueous solution in contact with the solid support, and allowed to bind to the first analyte binding region for a suitable incubation period (e.g., 1 hour). Next, the analyte solution can be removed, and a suitable buffer, such as phosphate buffered saline, containing a suitable oxygen scavenger system such as one containing 50 μg/mL protocatechuate-3,4-dioxygenase and 5 mM 3,4-dihydroxybenzoic acid, can be introduced under constant flow. The disappearance of the analyte from the surface as a function of incubation time can be measured by TIRF microscopy under illumination from a suitable laser source (e.g., 640 nm continuous wave laser) and with a suitable detector (e.g., a scientific complementary oxide semiconductor, sCMOS, sensor), and the remaining signal from surface-bound analyte as a function of time can be fit to a single-exponential decay function of the form

$$y = A * {e}^{- {k}_{d i s s o c} t}$$

where A is a constant, y is the amount of fluorescent signal from the surface-bound analyte, e is the base of the natural logarithm, and t is time. While the above equation is suitable for measurements of first-order reactions with low background signal, it will be appreciated by one skilled in the art that other, related exponential equations may be used in the case of kinetics that exhibit multiple rate constants (i.e., multi-exponential equations), or in cases where there is significant background signal (i.e., equations with a constant term added to the exponential term). It should also be understood that the same rate constant can be measured by displaying the non-fluorescently labeled analyte on the surface, and instead monitoring the dissociation of a fluorescently labeled first analyte binding region from the surface, with the caveat that any rate constant may be perturbed by changes to surface chemistry (e.g., altering which binding partner is immobilized) or labeling chemistry. It is preferable to measure any relevant rate constants in conditions resembling the final assay conditions as closely as possible, including the composition of the binding partners, buffer conditions, and temperature.

In certain aspects, the binding of the analyte to the first analyte binding region can be characterized as stable. It should be understood that this “stability” refers to the slower rate of dissociation between the first analyte binding region and the analyte versus between the second analyte binding region and the analyte. In any of the foregoing embodiments, the affinity between the first analyte binding region and the analyte can include a rate constant of dissociation that is ten (10)-fold slower than between the second analyte binding region and the analyte. By way of example, but not limitation, the affinity between the first analyte binding region and the analyte can include a rate constant of dissociation that is at least ten (10)-fold, fifteen (15)-fold, twenty (20)-fold, twenty-five (25)-fold, fifty (50)-fold, or one hundred (100)-fold slower than between the second analyte binding region and the analyte.

It should be understood that in any of the embodiments of this disclosure, the first analyte binding region can be any region that can sufficiently bind to the analyte. By way of example, but not limitation, where the analyte is a nucleic acid, the first analyte binding region can include a nucleic acid sequence that is complementary to the analyte oligonucleotide or DNA or RNA fragment or can include a DNA- or RNA-binding protein such as a zinc-finger domain, dead Cas9 enzyme (dCas9), ribonucleoprotein complex, or a methylated DNA-binding protein domain. By way of further example, but not limitation, where the analyte is a protein, peptide, or oligopeptide, the first analyte binding region can include a first binding partner, such as a first antibody or fragment thereof, a nucleic acid or peptide aptamer, or a ligand of the analyte with affinity to the analyte. In such embodiments, the first analyte binding region can be bound to a support and the signal region of the first probe can be bound to the support at an adjacent site. By way of further example, but not limitation, where the analyte is associated with another component, such as biotin, the first analyte binding region can associate with the component associated with the analyte, such as, in the case of a biotinylated analyte, streptavidin. Alternatively, the first analyte binding domain may comprise a biotinylated capture oligonucleotide that is biotinylated. This capture oligonucleotide may further comprise the signal regions of the first probe or sequences that allowing hybridization of separate oligonucleotides carrying the signal regions. It should be understood that in any embodiment in the disclosure that employs a streptavidin-biotin interaction, that other related proteins that interact with biotin with high affinity and specificity, such as avidin and NeutrAvidin, may be substituted in place of streptavidin. By way of example, but not limitation, the first analyte binding region can be bound to the nanoparticle through a streptavidin-biotin interaction where the first analyte binding region is biotinylated and the nanoparticle is coated with streptavidin. By way of further example, but not limitation, where the analyte is a small molecule such as a carbohydrate such as a polysaccharide or oligosaccharide, the first analyte binding region can include a capture antibody or an aptamer capable of binding the analyte; similarly, the second analyte binding can include an antibody or aptamers capable of binding the analyte.

In any of the foregoing embodiments, where the analyte is a nucleic acid, the first analyte binding region can include a first capture oligonucleotide that can include a sequence that is complementary to a sequence of the analyte. In embodiments where the first analyte binding region includes a first capture oligonucleotide, the first analyte binding region and the signal region of the first probe can be bound to adjacent sites on a support as described herein, where the support, for example, is coated with streptavidin and the first capture oligonucleotide can be biotinylated such that the first capture oligonucleotide is bound to the support through the streptavidin-biotin interaction. Alternatively, where the analyte is a nucleic acid linked to a binding partner, the binding partner can possess affinity to the first analyte binding region.

In any of the foregoing embodiments, the first capture oligonucleotide can have a length from about 8 nucleotides to about 50 nucleotides. By way of example, but not limitation, the first capture oligonucleotide can have a length from about 8 nucleotides to about 50 nucleotides, about 8 nucleotides to about 40 nucleotides, about 8 nucleotides to about 30 nucleotides, about 8 nucleotides to about 25 nucleotides, about 8 nucleotides to about 20 nucleotides, about 8 nucleotides to about 15 nucleotides, about 8 nucleotides to about 10 nucleotides, about 10 nucleotides to about 50 nucleotides, about 10 nucleotides to about 40 nucleotides, about 10 nucleotides to about 30 nucleotides, about 10 nucleotides to about 25 nucleotides, about 10 nucleotides to about 20 nucleotides, about 10 nucleotides to about 15 nucleotides, about 15 nucleotides to about 50 nucleotides, about 15 nucleotides to about 40 nucleotides, about 15 nucleotides to about 30 nucleotides, about 15 nucleotides to about 25 nucleotides, about 15 nucleotides to about 20 nucleotides, about 20 nucleotides to about 50 nucleotides, about 20 nucleotides to about 40 nucleotides, about 20 nucleotides to about 30 nucleotides, about 20 nucleotides to about 25 nucleotides, about 25 nucleotides to about 50 nucleotides, about 25 nucleotides to about 40 nucleotides, about 25 nucleotides to about 30 nucleotides, about 30 nucleotides to about 50 nucleotides, about 30 nucleotides to about 40 nucleotides, about 40 nucleotides to about 50 nucleotides, about 8, 10, 15, 20, 25, 30, 40, or 50 nucleotides.

In any of the foregoing embodiments, where the analyte is a nucleic acid linked to a binding partner, the binding partner can possess affinity for the first analyte binding region. By way of example but not limitation, the binding partner can be biotin and the first analyte binding region can include streptavidin.

In any of the foregoing embodiments, the first analyte binding region can be on the surface of a support. In such embodiments, the signal region can be bound to an adjacent site on the nanoparticle.

In any of the embodiments of the disclosure, where a capture oligonucleotide is included in the first probe, the capture oligonucleotide can include modifications that increase stability or specificity of the capture oligonucleotide, or which increase or decrease the affinity of the capture oligonucleotide for the analyte. By way of example, but not limitation, the modification can include LNA nucleotides, UNA nucleotides, PNA nucleotides, 2′-O-methyl-RNA nucleotides, 2′-fluororibonucleotides, phosphorothioate modifications, and combinations thereof.

In any of the foregoing embodiments, the first probes can each include one or more first analyte binding regions.

In any of the foregoing embodiments, the first analyte binding region can bind to one or more analytes.

### Signal Region

In any of the foregoing embodiments of the present disclosure, the first probe can include a first analyte binding region and a signal region.

In any of the foregoing embodiments, the signal region can be associated with a plurality of suppressors. In certain aspects, the signal region can be associated with a plurality of label probes in addition to the suppressors, where the suppressors prevent detection of the label probes when both are associated with the signal region. By way of example but not limitation, the label probes can be labeled with a fluorophore and the suppressors can be labeled with a quencher that reduces or prevents the generation of fluorescence signal from the fluorophore when the suppressors and label probes are both associated with the signal region. Alternatively, a plurality of label probes can be associated with the signal region after the removal or degradation of one or more suppressors. It should be understood that each label probe can associate with the signal region via a monovalent or multivalent mode of binding, e.g. via one, two, or more distinct moieties, each of which interacts with the signal region independently or in a cooperative fashion. By way of example, but not limitation, a label probe, such as a label oligonucleotide, can form one, two or more discrete double-stranded interactions with one or more components, such as oligonucleotide components, e.g. the first probe oligonucleotides, of the signal region. By way of further example, but not limitation, removal of one suppressor can be necessary for the binding of the label probe. By way of further example, but not limitation, two or more suppressors can be necessary for the binding of the label probe. By way of still further example, but not limitation, two or more adjacent suppressors can be necessary for the binding of the label probe.

In any of the foregoing embodiments, the first probes can include one or more signal regions. In some embodiments the first probe may include from about 1 to about 20 signal regions. In some embodiments, the first probe may include 2, 3, 4, 5, 6, 7, 8, 9, 10, 11, 12, 13, 14, 15, 16, 17, 18, or 19 signal regions.

In some embodiments, the signal regions may comprise an anchor oligonucleotide and a plurality of signal oligonucleotides, wherein each signal oligonucleotide comprises a first sequence that is complementary to a sequence on the anchor oligonucleotide and a second sequence that is complementary to the sequence of the suppressor oligonucleotides (but it not complementary to any sequence on the anchor oligonucleotide). In this way, multiple signal oligonucleotides (or signal regions) may be associated with the anchor oligonucleotide. In some embodiments, the anchor oligonucleotide may be a sequence on the capture oligonucleotide. In some instances, an anchor oligonucleotide is not necessary and the signal oligonucleotides comprise sequence that is complementary to sequence on the analyte. In this way, multiple signal regions (first probes) may be directly associated with a single copy of the analyte.

In some embodiments, the signal region can include reactive groups that can be blocked by the suppressors and which can subsequently be reacted after removal or degradation of the suppressors. By way of example but not limitation, the reactive groups can be azides, alkynes, dibenzocyclooctynes or other azide-reactive click chemistry moieties, trans-cyclooctenes, tetrazines, methyltetrazines, thiols, or amines. Preferably, the reactive groups have high stability in aqueous solution near neutral pH.

In some embodiments, the signal region can include a plurality of first probe oligonucleotides each comprising a common sequence. In such embodiments, the common sequence can be from about 10 nucleotides to about 50 nucleotides. As few as 10 nucleotides can be used if modified nucleotides, such as locked nucleic acids (LNA) or peptide nucleic acids (PNA) are incorporated into the sequence. Generally, for DNA with typical nucleotide abundances (about 25-75% GC content), the smallest feasible sequence would generally be at least 15-20 nucleotides in length at room temperature and in commonly used buffers. By way of example, but not limitation, the common sequence can be from about 10 nucleotides to about 50 nucleotides, about 14 nucleotides to about 50 nucleotides, about 15 nucleotides to about 50 nucleotides, about 20 nucleotides to about 50 nucleotides, about 30 nucleotides to about 50 nucleotides, about 40 nucleotides to about 50 nucleotides, about 10 nucleotides to about 40 nucleotides, about 10 nucleotides to about 30 nucleotides, about 10 nucleotides to about 25 nucleotides, about 10 nucleotides to about 20 nucleotides, about 14 nucleotides to about 40 nucleotides, about 14 nucleotides to about 25 nucleotides, about 14 nucleotides to about 20 nucleotides, about 15 nucleotides to about 40 nucleotides, about 15 nucleotides to about 30 nucleotides, about 15 nucleotides to about 25 nucleotides, about 15 nucleotides to about 20 nucleotides, about 20 nucleotides to about 40 oligonucleotides, about 20 nucleotides to about 30 nucleotides, about 20 nucleotides to about 25 nucleotides, about 25 nucleotides to about 40 nucleotides, about 25 nucleotides to about 30 nucleotides, about 30 nucleotides to about 40 nucleotides, about 10, 14, 15, 20, 25, 30, 40, or 50 nucleotides. By way of example, but not limitation, the signal region can include about 1 to about 50 first probe oligonucleotides. By way of further example, but not limitation, the signal region preferably includes 20 or fewer first probe oligonucleotides. By way of still further example, but not limitation, the signal region can include about 1 to about 50 first probe oligonucleotides, about 1 to about 40 first probe oligonucleotides, about 1 to about 30 first probe oligonucleotides, about 1 to about 25 first probe oligonucleotides, about 1 to about 20 first probe oligonucleotides, about 1 to about 15 first probe oligonucleotides, about 1 to about 10 first probe oligonucleotides, about 1 to about 5 first probe oligonucleotides, about 2 to about 50 first probe oligonucleotides, about 2 to about 40 first probe oligonucleotides, about 2 to about 30 first probe oligonucleotides, about 2 to about 25 first probe oligonucleotides, about 2 to about 20 first probe oligonucleotides, about 2 to about 15 first probe oligonucleotides, about 2 to about 10 first probe oligonucleotides, about 2 to about 5 first probe oligonucleotides, about 3 to about 50 first probe oligonucleotides, about 3 to about 40 first probe oligonucleotides, about 3 to about 30 first probe oligonucleotides, about 3 to about 25 first probe oligonucleotides, about 3 to about 20 first probe oligonucleotides, about 3 to about 15 first probe oligonucleotides, about 3 to about 10 first probe oligonucleotides, about 3 to about 5 first probe oligonucleotides, about 4 to about 50 first probe oligonucleotides, about 4 to about 40 first probe oligonucleotides, about 4 to about 30 first probe oligonucleotides, about 4 to about 25 first probe oligonucleotides, about 4 to about 20 first probe oligonucleotides, about 4 to about 15 first probe oligonucleotides, about 4 to about 10 first probe oligonucleotides, about 4 to about 5 first probe oligonucleotides, about 5 to about 50 first probe oligonucleotides, about 5 to about 40 first probe oligonucleotides, about 5 to about 30 first probe oligonucleotides, about 5 to about 25 first probe oligonucleotides, about 5 to about 20 first probe oligonucleotides, about 5 to about 15 first probe oligonucleotides, about 5 to about 10 first probe oligonucleotides, about 6 to about 50 first probe oligonucleotides, about 6 to about 40 first probe oligonucleotides, about 6 to about 30 first probe oligonucleotides, about 6 to about 25 first probe oligonucleotides, about 6 to about 20 first probe oligonucleotides, about 6 to about 15 first probe oligonucleotides, about 6 to about 10 first probe oligonucleotides, about 7 to about 50 first probe oligonucleotides, about 7 to about 40 first probe oligonucleotides, about 7 to about 30 first probe oligonucleotides, about 7 to about 25 first probe oligonucleotides, about 7 to about 20 first probe oligonucleotides, about 7 to about 15 first probe oligonucleotides, about 7 to about 10 first probe oligonucleotides, about 8 to about 50 first probe oligonucleotides, about 8 to about 40 first probe oligonucleotides, about 8 to about 30 first probe oligonucleotides, about 8 to about 25 first probe oligonucleotides, about 8 to about 20 first probe oligonucleotides, about 8 to about 15 first probe oligonucleotides, about 8 to about 10 first probe oligonucleotides, about 9 to about 50 first probe oligonucleotides, about 9 to about 40 first probe oligonucleotides, about 9 to about 30 first probe oligonucleotides, about 9 to about 25 first probe oligonucleotides, about 9 to about 20 first probe oligonucleotides, about 9 to about 15 first probe oligonucleotides, about 9 to about 10 first probe oligonucleotides, about 10 to about 50 first probe oligonucleotides, about 10 to about 40 first probe oligonucleotides, about 10 to about 30 first probe oligonucleotides, about 10 to about 25 first probe oligonucleotides, about 10 to about 20 first probe oligonucleotides, about 10 to about 15 first probe oligonucleotides, about 15 to about 20 first probe oligonucleotides, about 20 to about 50 first probe oligonucleotides, about 20 to about 40 first probe oligonucleotides, about 20 to about 30 first probe oligonucleotides, about 20 to about 25 first probe oligonucleotides, about 25 to about 50 first probe oligonucleotides, about 25 to about 40 first probe oligonucleotides, about 25 to about 30 first probe oligonucleotides, about 30 to about 50 first probe oligonucleotides, about 30 to about 40 first probe oligonucleotides, about 40 to about 40 first probe nucleotides, or about 1, 2, 3, 4, 5, 6, 7, 8, 9, 10, 11, 12, 13, 14, 15 16, 17, 18, 19, 20, 25, 30, 40, 50 or more first probe oligonucleotides.

In any of the foregoing embodiments, where the signal region includes nucleic acids, such as, by way of example but not limitation, where the signal region includes a plurality of first probe oligonucleotides, the signal region can have any suitable length; preferably the length of any contiguous nucleic acid molecule within the signal region is 200 nucleotides or smaller. By way of example, but not limitation, in such embodiments, the signal region can be from about 10 to about 200 nucleotides, about 10 to about 150 nucleotides, about 10 to about 100 nucleotides, about 10 to about 50 nucleotides, about 10 to about 40 nucleotides, about 10 to about 30 nucleotides, about 10 to about 25 nucleotides, about 10 to about 20 nucleotides, about 20 to about 200 nucleotides, about 20 to about 150 nucleotides, about 20 to about 100 nucleotides, about 20 to about 50 nucleotides, about 20 to about 40 nucleotides, about 20 to about 30 nucleotides, about 20 to about 25 nucleotides, about 25 to about 200 nucleotides, about 25 to about 150 nucleotides, about 25 to about 100 nucleotides, about 25 to about 50 nucleotides, about 25 to about 40 nucleotides, about 25 to about 30 nucleotides, about 30 to about 200 nucleotides, about 30 to about 150 nucleotides, about 30 to about 100 nucleotides, about 30 to about 50 nucleotides, about 30 to about 40 nucleotides, about 40 to about 200 nucleotides, about 40 to about 150 nucleotides, about 40 to about 100 nucleotides, about 40 to about 50 nucleotides, about 50 to about 200 nucleotides, about 50 to about 150 nucleotides, about 50 to about 100 nucleotides, about 100 to about 200 nucleotides, about 100 to about 150 nucleotides, about 150 to about 200 nucleotides, about 10, 20, 25, 30, 40, 50, 100, 150, 200 or more nucleotides.

### Suppressors

In any of the foregoing embodiments, the signal region can be associated with a plurality of suppressors.

By way of example, but not limitation, each suppressor can include a DNA oligonucleotide, a locked nucleic acid (LNA) oligonucleotide, a peptide nucleic acid (PNA) oligonucleotide, an RNA oligonucleotide, a chemical protecting group such as tert-butyloxycarbonyl (Boc) or fluorenylmethoxycarbonyl (Fmoc) that suppresses the reactivity of chemical groups on the signal region, an endonuclease cleave site, a peptide sequence, a protease cleavage site, a protein subunit, an enzyme inhibitor, or a fluorescence quencher such as a Black Hole Quencher or Iowa Black Quencher. In some embodiments, the suppressor can be a bulky polymer substituent that can be a substrate for an enzyme.

In any of the foregoing embodiments, where the signal region includes a plurality of first probe oligonucleotides each comprising a common sequence, the suppressors can each include a suppressor oligonucleotide which can include a first probe complementary region and, optionally, a toehold region, where the first probe complementary region includes a sequence that is complementary to the common sequence of the first probe oligonucleotides, where the toehold region is located immediately 3′ or 5′ from the first probe complementary region and is not complementary to sequence immediately 3′ or 5′ of the common sequence on the first probe oligonucleotides such that when the suppressor oligonucleotides are hybridized to the first probe oligonucleotides, the toehold regions do not hybridize to the first probe oligonucleotides. In such embodiments, the first probe complementary region can have the same length as the common sequence. In such embodiments, the toehold region, if present, can include from about 1 nucleotide to about 6 nucleotides. By way of example, but not limitation, the toehold region can include from about 1 nucleotide to about 6 nucleotides, about 2 nucleotides to about 5 nucleotides, about 2 nucleotides to about 4 nucleotides, about 3 nucleotides to about 6 nucleotides, about 3 nucleotides to about 5 nucleotides, about 3 nucleotides to about 4 nucleotides, about 4 nucleotides to about 6 nucleotides, about 4 nucleotides to about 5 nucleotides, about 5 nucleotides to about 6 nucleotides, about 1, 2, 3, 4, 5, or 6 nucleotides. In some embodiments, the length, sequence, and/or free energy of binding of the toehold is chosen to yield a measured rate constant of toehold-mediated strand displacement of about 1 M⁻¹ s⁻¹ to about 1000 M⁻¹ s⁻¹ according to the principles taught by Zhang and Winfree, “Control of DNA Strand Displacement Kinetics Using Toehold Exchange,” J. Am. Chem. Soc. 131 (47): 17303-17314 (2009), which is incorporated herein by reference in its entirety.

### Second Probes

In any of the embodiments of the present disclosure, the second probes can, via the suppressor interacting region, indirectly or directly be capable of binding to, removing or degrading one of the suppressors. By way of example, but not limitation, the second probe can include one or more DNA oligonucleotides, one or more RNA oligonucleotides, one or more locked nucleic acid (LNA) oligonucleotides, one or more peptide nucleic acid (PNA) oligonucleotides, one or more antibodies or antibody fragments, one or more aptamers, an enzyme, a nuclease, a restriction endonuclease, an RNase, an RNase H, a Cas9 family nuclease, a protease, a transferase enzyme, a horseradish peroxidase or mutant thereof, or a combination thereof. It should be understood that each of these elements can be or be a part of the second analyte binding region or the suppressor interacting region.

In certain aspects, the second probes can, via the suppressor interacting region, directly be capable of binding to, removing or degrading one of the suppressors. By way of example, but not limitation, the suppressor interacting region can be capable of binding to, removing or degrading one the suppressors via complementarity or enzymatic action.

In certain aspects, the second probes can, by binding to the analyte, create a binding site for a third probe that can complete the removal of the suppressor, where the third probe can only remove a suppressor with high probability if the second probe is present at a locally high concentration. The binding site for the third probe could either be on the suppressor or could be on the first probe itself, but in either case, would cause displacement of the suppressor from the signal region. In the case where the third probe binds to the signal region, the third probe itself can include a label probe or label-probe binding site.

In certain aspects, the second probe can bind to the suppressor in a manner that creates an active site for an enzyme which acts as a third probe, the enzyme can then degrade the suppressor. By way of example, but not limitation, the enzyme can be an RNase H or restriction endonuclease.

In certain aspects, the second probe can include a necessary co-factor or co-enzyme for an enzyme, as a third probe, that can degrade or remove a suppressor. The presence of the co-factor or co-enzyme at a high local concentration near the second probe allows the enzyme to degrade a nearby suppressor. By way of example, but not limitation, the coenzyme can be NAD⁺/NADH tether to a flexible linker which can, in turn enable the activity of a nearby enzyme such as that described in Fu, et al., “Multi-enzyme complexes on DNA scaffolds capable of substrate channeling with an artificial swinging arm,” Nature Nanotech. 9:531-536 (2014).

In any of the embodiments of the present disclosure, the number of second probes can be in excess of the number of first probes. In any of the embodiments of the present disclosure, the number of second probes can be greater than the number of first probes associated with the analyte.

### Second Analyte Binding Region

In any of the embodiments of the present disclosure, where the first analyte binding region of each first probe includes a first binding partner with affinity to the analyte, the second analyte binding region can include a second binding partner with affinity to the analyte, where the suppressor interacting region is linked to the second binding partner. By way of example, but not limitation, the second binding partner can be an antibody or fragment thereof, a single-chain variable fragment, a nanobody, a peptide aptamer, a nucleic acid aptamer, a small molecule, a metabolite, an oligopeptide sequence, a polypeptide, a nucleic acid, or any other ligand with affinity for the analyte.

In any of the foregoing embodiments, where the analyte is a nucleic acid and the first analyte binding region can include a first capture oligonucleotide that can include a sequence that is complementary to a first sequence of the analyte, the second analyte binding region can include a detection oligonucleotide that has a sequence that is complementary to a second sequence of the analyte. It should be understood that instead of a detection oligonucleotide, the second analyte binding region can include a detection portion such as, by way of example, but not limitation, a DNA- or RNA-binding protein, a catalytically dead Cas9 enzyme (dCas9)-guide RNA complex, an RNA-induced silencing complex (RISC), a ribonucleoprotein complex, an antibody, an aptamer, or a methylated DNA-binding protein domain (MBD). It is expected, without being bound to theory that the kinetic nature of the approach will allow facile discrimination between a mutant DNA or RNA sequence and related sequences, such as a wild-type sequence. In any of the foregoing embodiments, where the analyte is a nucleic acid, the second analyte binding region may exhibit distinct binding or dissociation kinetics when binding to the analyte compared to when binding to a similar sequence differing from the analyte by one or more nucleotides or by the presence or absence of chemical modifications such as, by way of example but not limitation, 5-methylcytosine or N⁶-methyladenosine, distinct from those present or absent in the analyte sequence. By way of example but not limitation, the second analyte binding region may dissociate more slowly (e.g., more than about 2-fold, about 5-fold, or about 10-fold more slowly) from an analyte sequence than from a similar sequence. By way of example but not limitation, the second analyte binding region may dissociate more rapidly (e.g., more than about 2-fold, about 5-fold, or about 10-fold more rapidly) from an analyte sequence than from a related sequence. In such embodiments, the second analyte binding region may distinguish between the analyte and a related sequence by exhibiting different kinetics of dissociation from the analyte than from the related sequence and, as a result, yield a different extent of suppressor removal from the first probe in the presence of the analyte than in the presence of the related sequence. In such embodiments, the presence of the analyte can thus be distinguished from the presence of the related sequence by the presence or absence of sufficiently bright puncta following the removal of suppressors. In any of the foregoing embodiments, where the second analyte binding region includes a detection oligonucleotide, the length and sequence of the detection oligonucleotide can be designed so as to achieve a significant difference in kinetics of binding or dissociation with the analyte compared to a related sequence according to principles such as those outlined for query probes in the techniques in Hayward, S. L. et al. “Ultraspecific and Amplification-Free Quantification of Mutant DNA by Single-Molecule Kinetic Fingerprinting,” J. Am. Chem. Soc. 140, 11755-11762 (2018), and Johnson-Buck, A. et al., “A guide to nucleic acid detection by single-molecule kinetic fingerprinting,” Methods 153, 3-12 (2019), both of which are incorporated herein by reference in their entirety. In any of the foregoing embodiments, where the analyte is a nucleic acid, where the second analyte binding region includes a detection oligonucleotide, one or more competitor probes comprising a nucleic acid sequence complementary to the related sequence, but with less complementarity to the analyte than to the related sequence, may be added during the incubation step with the second probe, to reduce the extent of binding of the second analyte binding region to the related sequence. The design and use of such competitor probes is taught by, for example, Hayward, S. L. et al. “Ultraspecific and Amplification-Free Quantification of Mutant DNA by Single-Molecule Kinetic Fingerprinting,” J. Am. Chem. Soc. 140, 11755-11762 (2018).

### Suppressor Interacting Region

In any of the foregoing embodiments, the suppressor interacting region can be any region that is capable of directly or indirectly binding to, removing, or degrading one or more of the suppressors.

In any of the foregoing embodiments, where the suppressors each include a suppressor oligonucleotide which can include a first probe complementary region and, optionally, a toehold region, the suppressor interacting region of the second probe can include a second probe oligonucleotide that includes a suppressor complementary region that includes a sequence that is complementary to at least a portion, if not all of, the first probe complementary region and, if present, to at least a portion, if not all of, the toehold region of the suppressor oligonucleotide. Due to the complementarity of the suppressor complementary region to the suppressor oligonucleotide, the suppressor interacting region is able to associate with the suppressor and remove it from the signal region of the first probe. In some embodiments, the length, sequence, and/or free energy of binding of the suppressor interacting region to a suppressor oligonucleotide is chosen to yield a measured rate constant of strand displacement of about 1 M⁻¹ s⁻¹ to about 1000 M⁻¹ s⁻¹ according to the principles taught by Zhang and Winfree, “Control of DNA Strand Displacement Kinetics Using Toehold Exchange,” J. Am. Chem. Soc. 131 (47): 17303-17314 (2009).

It should be understood that the mechanism of removal or degradation of a suppressor from the signal region can occur by various mechanisms. By way of example, but not limitation, the mechanism of removal or degradation of one or more suppressors from the signal region can be Watson-Crick base pairing, non-canonical base pairing, toehold-mediated strand displacement (TMSD), toehold exchange, degradation or partial degradation by an endonuclease or exonuclease, RNA or DNA helicase activity, nucleophilic substitution (enzymatic or non-enzymatic), hydrolysis (enzymatic or non-enzymatic), transamination (enzymatic or non-enzymatic), or disulfide exchange (enzymatic or non-enzymatic).

While the present disclosure generally discloses systems where the second probe can include a second analyte binding region and a suppressor interacting region, it should be understood that in alternative embodiments, where the first probe comprises an aptamer, the second probe can include a first probe binding region and a suppressor interacting region rather than the second analyte binding region and the suppressor interacting region. In such embodiments, upon binding of the analyte to the first analyte binding region of the first probe, the first probe can undergo a conformational change that exposes or creates a binding site for the first probe binding region. By way of example, but not limitation, the first probe can include an aptamer that can undergo such a conformational change upon binding of the analyte. By way of further example, but not limitation, the first analyte binding region can include the aptamer such as, by way of example, but not limitation a nucleic acid aptamer. It should be understood that the kits and methods of the present disclosure can be modified to incorporate this system and that the kinetic relationship between first probe binding to the analyte and second probe binding to the analyte can be applied to first probe binding to the analyte and second probe binding to the first probe, such as via the first probe binding region to the first probe when it is bound to the analyte. By way of still further example, but not limitation, where the first probe, such as the first analyte binding region, includes an aptamer, the aptamer can include a nucleotide sequence that includes a flexible loop, where binding of the analyte to the aptamer can open the loop to expose the binding site for the first probe binding region of the second probe. It should be understood that the disclosure regarding the first probes, second probes, and other elements of the systems and methods of the present disclosure can be applied in such alternative embodiments.

By way of still further example, but not limitation, the first analyte binding region can include an aptamer that can change conformation upon the binding of an analyte, such as, by way of example but not limitation, a metal ion, a small organic molecule, a monosaccharide, polysaccharide or oligosaccharide, mononucleotide, dinucleotide, oligonucleotide or polynucleotide, amino acid, peptide or protein, which results in the creation or modification of a binding site for a first probe binding region of the second probe, by way of example but not limitation, a nucleotide sequence that is complementary to the binding site that is created upon binding of the analyte. Exemplary aptamer-based probes that can change conformation to enable detection by a complementary probe are described in detail in Weng, et al., “Single-Molecule Kinetic Fingerprinting for the Ultrasensitive Detection of Small Molecules with Aptasensors,” Anal. Chem. 2019, 91, 2, 1424-1431, which is incorporated herein by reference in its entirety. In any of these alternative embodiments, the aptamer can be a nucleic acid aptamer.

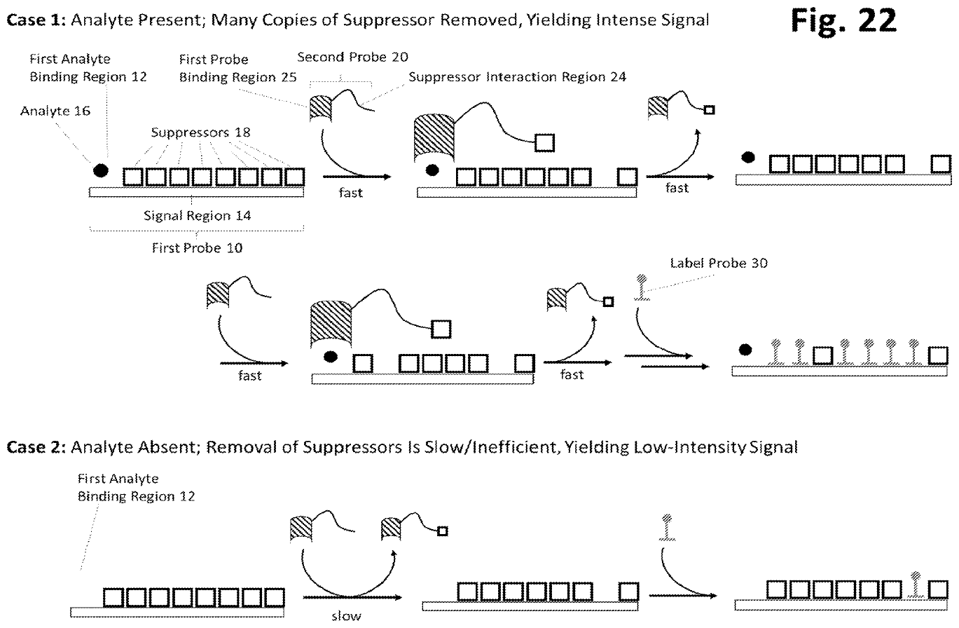

An exemplary system where the second probe can bind the first probe when it is bound to the analyte is depicted in FIG. 22. Case 1 of FIG. 22 depicts an exemplary system where the first probe 10 includes a first analyte binding region 12 for binding at least one analyte 16 and a signal region 14 which is associated with a plurality of suppressors 18. A second probe 20 that includes a first probe binding region 25 and a suppressor interacting region 24 can quickly, relative to the dissociation of the analyte 16 from the first analyte binding region 12, bind to the first probe via the first probe binding region 25 and directly or indirectly remove or degrade one or more of the suppressors 18. This binding can increase the local concentration of the second probe 20 significantly and increase the rate of suppressor removal or degradation by a second probe bound to the first probe via the first probe binding region relative to the rate of suppressor removal or degradation by a second probe not bound via the first probe binding region. By having a more transient interaction between the first probe binding region 25 and the first probe 10 than between the first analyte binding region and the analyte, multiple copies of the suppressors 18 can be removed quickly by multiple successive binding events of second probes to the first probe, which can result in a more intense signal than in the absence of analyte as discussed below. By removing the one or more suppressors 18, binding sites are exposed for the label probe 30 which can then bind where the suppressors were removed and can then be used for signal detection. As shown in Case 2 of FIG. 22, in the absence of analyte, the first analyte binding region 12 lacks a well-formed binding site for the first probe binding region 25. As a result, the removal of suppressors 18 by the second probe 20 is slow because the local concentration of the second probe 20 is not enhanced by its binding to the first analyte binding region via the first probe binding region 25, which results in a slow and inefficient reaction that yields a low intensity signal.

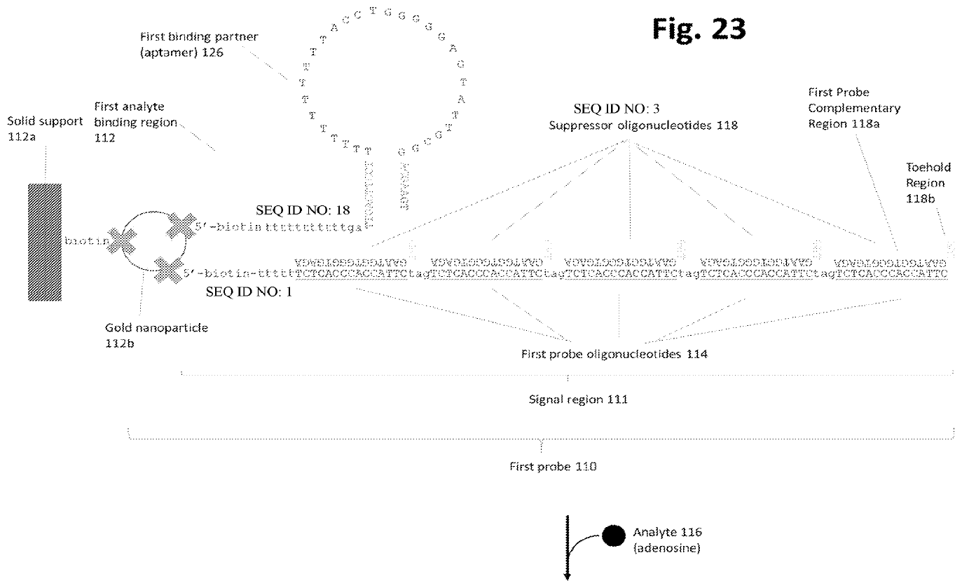

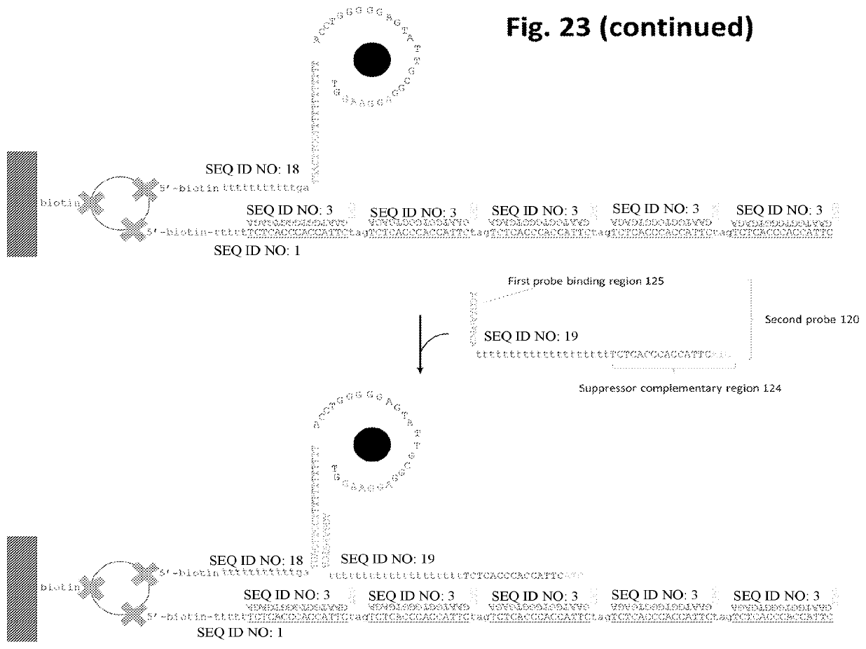

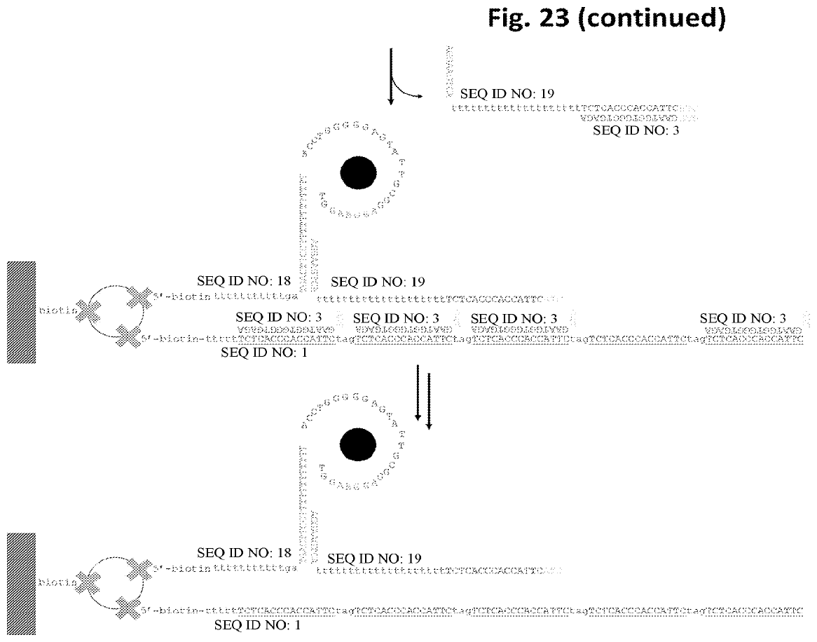

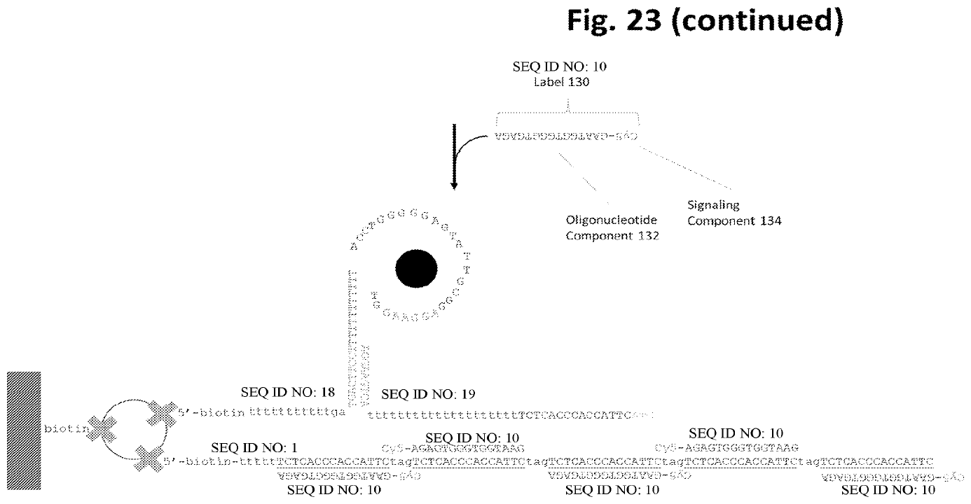

A further exemplary embodiment is depicted in FIG. 23 where the first analyte binding region includes an aptamer that can change conformation upon analyte binding, allowing for a second probe to bind to the first probe. FIG. 23 depicts a system in which a small-molecule analyte (adenosine) 116 is detected via its interaction with a DNA aptamer sequence (first binding partner, 126) within the first analyte binding region 112. Binding of the analyte 116 to the aptamer 126 exposes a binding site for the first probe binding region 125 of the second probe 120 via Watson-Crick base pairing. Upon binding of the first probe binding region 125 to the first analyte binding region 112, the second probe 120 is present at a high local concentration, leading to the efficient removal of a suppressor 118 (which includes a first probe complementary region 118a which is complementary to the first probe oligonucleotides 114 of the signal region 111 of the first probe 110) and a toehold region 118b) from the signal region of the first probe by toehold-mediated strand displacement via the suppressor complementary region 124. Due to the transient nature of the binding of the second probe 120 to the first analyte binding region 112, the second probe 120 quickly dissociates from the first probe 110. After the sequential binding and removal of suppressors 118 by multiple copies of the second probe 120, multiple first probe oligonucleotides 114 are made available for the label probe 130 (which includes an oligonucleotide component 132 and signaling component 134) to bind to. The binding of multiple label probes 130 to the signal region 111 of the first probe 110 yields a bright signal indicating the presence of the analyte. The sequence of the first analyte binding region, including the aptamer and binding site for the first probe binding region, are taken from Weng, et al., “Single-Molecule Kinetic Fingerprinting for the Ultrasensitive Detection of Small Molecules with Aptasensors,” Anal. Chem. 2019, 91, 2, 1424-1431. In this exemplary embodiment, the signal region and the first analyte binding region are bound to a gold nanoparticle 112b by streptavidin-biotin interaction and the first probe is bound to a solid support 112a via a streptavidin-biotin interaction.

### Label Probes

In any of the embodiments of the present disclosure, the label probes can include a signaling component. The label probes can be associated with the signal region, where the suppressors can prevent a signal from the label probes, or the label probes can be capable of binding to the signal region once one or more suppressors are removed or degraded.

In any of the embodiments of the present disclosure, where the suppressors include a first probe complementary region, the label probes can include a signaling component and an oligonucleotide component, where the oligonucleotide component includes a sequence identical to the first probe complementary region of the suppressor or a portion of such sequence.

In any of the embodiments of the present disclosure, the signaling component can be any suitable signaling component. By way of example, but not limitation, the signaling component can be a fluorophore, a fluorogenic compound, a fluorogenic enzyme substrate, a fluorescent protein, or a chemiluminescence-generating enzyme. By way of further example, but not limitation, the signaling component can be a small organic fluorophore selected from the group consisting of Cy3, Cy5, Cy5.5, Cy7, ATTO 647N, DyLight 550, DyLight 650, Alexa Fluor 488, Alexa Fluor 555, and Alexa Fluor 647.

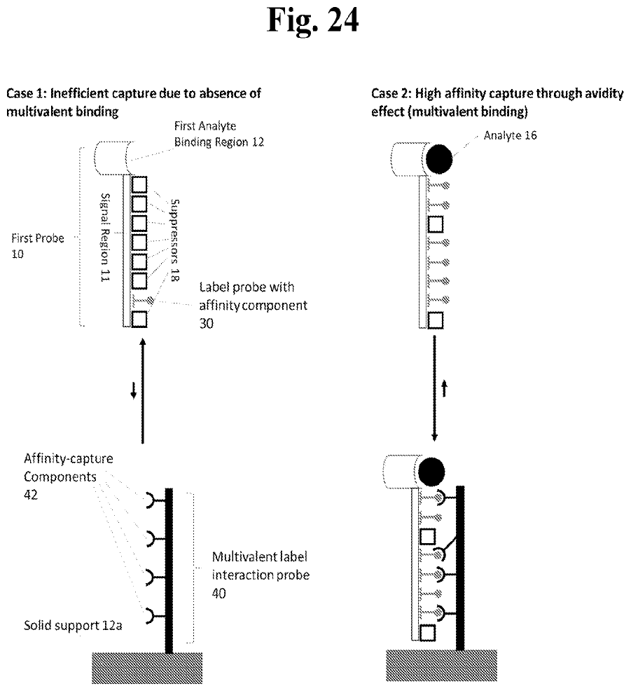

In any of the embodiments of the present disclosure, the label probe can further include an affinity component that has specific affinity for an affinity-capture component or can include such affinity component instead of the signaling component. In some embodiments, the interaction between the affinity component and the affinity-capture component is transient or unstable at ambient temperature, but rendered stable through the avidity effect if multiple labels present on the same signaling region bind to multiple affinity-capture components on the same surface or other object, such as a colloidal particle, nanoparticle, or protein. Under such circumstances, only signaling regions bearing multiple copies of the label are likely to be stably bound by the affinity-capture component at the surface. In some embodiments, the affinity-capture component is displayed on an assay surface such as a microscope slide, coverslip, multiwell plate, or microfluidic chip. In some embodiments, the affinity-capture component is displayed on microparticle, a colloidal particle, bead, nanostructure, or biological moiety. In some embodiments, the affinity component comprises an oligonucleotide sequence that interacts with a complementary or partly complementary sequence of the surface-capture component. In some embodiments, the affinity component and affinity-capture component interact via between 3 and 12 base pairs. In some embodiments, the affinity component and affinity-capture component interact via blunt-end, sticky-end, or coaxial stacking of DNA or RNA duplexes. By using an affinity component/affinity-capture component system, the first probes can be captured at a surface after the label probes have been bound to the signaling region(s). In one exemplary embodiment, multivalent binding is used to selectively capture first probes from which multiple suppressors have been removed or degraded due to the presence of the analyte (FIG. 24). In Case 1, only a small number (e.g., 0 or 1) of suppressors 18 have been removed from the signal region 11 of the first probe 10 due to the inefficient interaction of a second probe with the first probe in the absence of the analyte, leading to the presence or accessibility of only a small number (e.g., 0 or 1) of label probes 30 associated with the first probe. Although the label probe or probes still bear an affinity component, their association with a solid support 12a or other component is rendered inefficient by the low-affinity interaction of individual affinity components with the individual affinity-capture components 42 present on a multivalent label interaction probe. As a result, few or no first probes bearing a small number of labels associate with the multivalent label interaction probe 40. In contrast, Case 2 depicts a first probe 10 from which multiple suppressors 18 have been removed or degraded due to the presence of the analyte 16, resulting in the binding of many label probes 30 to the signal region 11 of the first probe 10. As a result, the first probe 10 displays multiple copies of the affinity component, leading to multivalent interaction with the affinity-capture components 42 of the multivalent label interaction probe, which results in efficient, rapid, or high-affinity capture compared to Case 1. It should be understood that the multivalent label interaction probe can be used for capture, but can also be associated with a signaling component such that binding of the multivalent label interaction probe to label probes associated with a first probe can allow for detection of a signal as described in the present disclosure. It should be understood that the multivalent label interaction probe may be useful to allow the reactions between the components of the system to be performed in solution or suspension. This is exemplified further in Example 7 below. Again, as alluded to above, the label probes must include an additional sequence or region that permit interaction with the multivalent label interaction probe. It is particularly important in the multivalent capture context (although important in all embodiment described herein) that the second probe is in excess of the suppressor probe, and preferably at least two-fold excess. Furthermore, the second probe should be in excess of the first probe and the label probes should be in excess of the second probes.

### Kits

It should be understood that kits with components sufficient to carry out the methods of the present disclosure are encompassed within the present disclosure and can include the components necessary to practice the relevant method.

In some embodiments, a kit for detecting an analyte in a sample is provided that includes: a first component that includes a plurality of first probes, each first probe including a first analyte binding region and a signal region, where the first analyte binding region has affinity to the analyte; a second component that includes a plurality of label probes, where each label probe is able to associate with the signal region; a third component that includes a plurality of suppressors, where each suppressor is able to associate with the signal region, and where the suppressors prevent detection of the label probes when both are associated with the signal region or where the suppressors prevent association of the label probes with the signal region; and a fourth component that includes a plurality of second probes, where each second probe includes a second analyte binding region and a suppressor interacting region, where the second analyte binding region has affinity to the analyte, where the suppressor interacting region is indirectly or directly capable of binding to, removing, or degrading one or more of the suppressors, and where the interaction of the first analyte binding region to the analyte possesses a rate constant of dissociation that is slower than the rate constant of dissociation between the second analyte binding region and the analyte.

In any of the foregoing kits, the first component, second component, third component, and fourth component can be provided individually or in various combinations. By way of example, but not limitation, the first component and the third component can be provided in a first reaction mixture. In such embodiments, the second component and fourth component can be provided in separate reaction mixtures and are not part of the first reaction mixture. Alternatively, the second component and fourth component can be provided as a second reaction mixture. By way of further example, but not limitation, the first component, the second component and the third component can be provided in a first reaction mixture. In such embodiments, the fourth component cannot be a part of the first reaction mixture.

In any of the foregoing kit embodiments, the kit can further include a multivalent interaction probe as a fifth component or as a component of any of the other reaction mixtures of components of the kits disclosed herein.

It should be understood that the components of the kits of the present disclosure can have the features and characteristics of the corresponding elements of any of the embodiments of the present disclosure, including the various alternative configurations described for each of the various components described herein.

### Methods

In some embodiments, a method for detecting an analyte in a sample can include: (a) mixing at least a portion of the sample with a plurality of first probes, each first probe comprising a first analyte binding region and a signal region, where the first analyte binding region has affinity to the analyte, and a plurality of suppressors, where each suppressor is able to associate with the signal region, where the suppressors can prevent detection of a label probe when both are associated with the signal region or can prevent association of the label probe with the signal region, to form a first reaction mixture, where the suppressors are bound to the signal region of the plurality of first probes; (b) incubating the first reaction mixture under conditions sufficient for the analyte to bind to the first analyte binding region; (c) mixing the first reaction mixture following step (b) with a plurality of second probes, each second probe comprising a second analyte binding region and a suppressor interacting region, where the second analyte binding region has affinity to the analyte, where the suppressor interacting region is indirectly or directly capable of binding to, removing, or degrading one or more of the suppressors, and where the interaction of the first analyte binding region to the analyte possesses a rate constant of dissociation that is slower than the rate constant of dissociation between the second analyte binding region and the analyte, to yield a second reaction mixture; (d) incubating the second reaction mixture under conditions sufficient for the second probe to bind to and dissociate from the analyte and remove one or more suppressors from the signal region of the plurality of first probes or degrade one or more suppressors; (e) mixing the second reaction mixture following step (d) with a plurality of label probes, where each label probe is able to associate with the signal region to yield a third reaction mixture; (f) incubating the third reaction mixture under conditions sufficient to permit the label probe to associate with the signal regions of first probes that have had one or more suppressors removed or degraded in step (d); and (g) measuring a signal generated from the label probes associated with the signal regions of the first probe to determine the presence of the analyte in the sample.

It should be understood that in the foregoing methods, prior to step (a), the plurality of first probes and the plurality of suppressors can be mixed such that the suppressors are bound to the signal region of the plurality of first probes prior to adding the sample. It should also be understood that in the foregoing method, the plurality of first probes can be incubated with the analyte under conditions sufficient for the analyte to bind to the first analyte binding region prior to adding the plurality of suppressors and/or label probes. It should also be further understood that the plurality of label probes and plurality of second probes can be added simultaneously and need not be added and incubated in separate steps.

In some embodiments, a method for detecting an analyte in a sample can include: (a) mixing at least a portion of the sample with a plurality of first probes, each first probe comprising a first analyte binding region and a signal region, where the first analyte binding region has affinity to the analyte, each signal region is associated with a plurality of suppressors, where each suppressor is able to associate with the signal region, where the suppressors can prevent detection of a label probe when both are associated with the signal region or can prevent association of the label probe with the signal region, to form a first reaction mixture, where the suppressors are bound to the signal region of the plurality of first probes; (b) incubating the first reaction mixture under conditions sufficient for the analyte to bind to the first analyte binding region; (c) mixing the first reaction mixture following step (b) with a plurality of second probes, each second probe comprising a second analyte binding region and a suppressor interacting region, where the second analyte binding region has affinity to the analyte, where the suppressor interacting region is indirectly or directly capable of binding to, removing, or degrading one or more of the suppressors, and where the interaction of the first analyte binding region to the analyte possesses a rate constant of dissociation that is slower than the rate constant of dissociation between the second analyte binding region and the analyte, to yield a second reaction mixture; (d) incubating the second reaction mixture under conditions sufficient for the second probe to bind to and dissociate from the analyte and remove one or more suppressors from the signal region of the plurality of first probes or degrade one or more suppressors; (e) mixing the second reaction mixture following step (d) with a plurality of label probes, where each label probe is able to associate with the signal region to yield a third reaction mixture; (f) incubating the third reaction mixture under conditions sufficient to permit the label probe to associate with the signal regions of first probes that have had one or more suppressors removed or degraded in step (d); and (g) measuring a signal generated from the label probes associated with the signal regions of the first probe to determine the presence of the analyte in the sample.

In some embodiments, a method for detecting an analyte in a sample can include: (a) mixing at least a portion of the sample with a plurality of first probes, each first probe comprising a first analyte binding region and a signal region, where the first analyte binding region has affinity to the analyte, each signal region is associated with a plurality of suppressors, where each suppressor is able to associate with the signal region, where the suppressors can prevent detection of a label probe when both are associated with the signal region or can prevent association of the label probe with the signal region, to form a first reaction mixture, where the suppressors are bound to the signal region of the plurality of first probes; (b) incubating the first reaction mixture under conditions sufficient for the analyte to bind to the first analyte binding region; (c) mixing the first reaction mixture following step (b) with a plurality of second probes, each second probe comprising a second analyte binding region and a suppressor interacting region, where the second analyte binding region has affinity to the analyte, where the suppressor interacting region is indirectly or directly capable of binding to, removing, or degrading one or more of the suppressors, and where the interaction of the first analyte binding region to the analyte possesses a rate constant of dissociation that is slower than the rate constant of dissociation between the second analyte binding region and the analyte, and a plurality of label probes, where each label probe is able to associate with the signal region, to yield a second reaction mixture; (d) incubating the second reaction mixture under conditions sufficient for the second probe to bind to and dissociate from the analyte and remove one or more suppressors from the signal region of the plurality of first probes or degrade one or more suppressors and to permit the label probe to associate with the signal regions of first probes that have had one or more suppressors removed or degraded; and (e) measuring a signal generated from the label probes associated with the signal regions of the first probe to determine the presence of the analyte in the sample.

In some embodiments, a method for detecting an analyte in a sample can include: (a) mixing at least a portion of the sample with a plurality of first probes, each first probe comprising a first analyte binding region and a signal region, where the first analyte binding region has affinity to the analyte, each signal region is associated with a plurality of suppressors, where each suppressor is able to associate with the signal region, where the suppressors can prevent detection of a label probe when both are associated with the signal region or can prevent association of the label probe with the signal region, and a plurality of label probes, where each label probe is able to associate with the signal region, to form a first reaction mixture, where the suppressors and label probes are bound to the signal region of the plurality of first probes; (b) incubating the first reaction mixture under conditions sufficient for the analyte to bind to the first analyte binding region; (c) mixing the first reaction mixture following step (b) with a plurality of second probes, each second probe comprising a second analyte binding region and a suppressor interacting region, where the second analyte binding region has affinity to the analyte, where the suppressor interacting region is indirectly or directly capable of binding to, removing, or degrading one or more of the suppressors, and where the interaction of the first analyte binding region to the analyte possesses a rate constant of dissociation that is slower than the rate constant of dissociation between the second analyte binding region and the analyte, to yield a second reaction mixture; (d) incubating the second reaction mixture under conditions sufficient for the second probe to bind to and dissociate from the analyte and remove one or more suppressors from the signal region of the plurality of first probes or degrade one or more suppressors; and (e) measuring a signal generated from the label probes associated with the signal regions of the first probe to determine the presence of the analyte in the sample. In such embodiments, the label probes can be not associated with the signal region of the first reaction mixture and step (d) comprises conditions sufficient to permit the label probes to associate with the signal regions of the first probes that have had one or more suppressors removed. Alternatively, in such embodiments, the label probes can be associated with the signal region in the first reaction mixture.

In any of the foregoing methods, the method can further include a step of incubating the first probes associated with label probes with a multivalent label interaction probe that includes affinity-capture components which can bind to the affinity components of the label probes, if present, for either capture of the first probes with a sufficient number of label probes or for detection, where the multivalent label interaction probe can include a signaling component.

In any of the foregoing methods, the number of second probes can be in excess of the number of first probes.

It should be understood that in the foregoing methods, the step of measuring a signal generated from the label probes associated with the signal regions of the first probe to determine the presence of the analyte in the sample can be performed by various mechanisms including direct or indirect detection of the label probes. By way of example, but not limitation, measurement and/or quantification of the number of copies of suppressors removed from each first probe can be performed by fluorescence microscopy, total internal reflection fluorescence (TIRF) microscopy, epifluorescence microscopy, confocal fluorescence microscopy, flow cytometry, chemiluminescence, gel electrophoresis, size exclusion chromatography, gel filtration chromatography, affinity chromatography, adsorption, biding or conjugation of the first probes to a surface of a coverslip, microscope slide, or assay plate, where said adsorption, binding or conjugation is either enhanced or inhibited by the removal of suppressors. By way of further example, where the signal region can include a reactive group on the signal region that is blocked by the suppressors, upon removal or degradation of one or more suppressors, the reactive group can be reacted with a reagent bearing a corresponding reactive group and a signaling component such as a fluorophore. By way of still further example, but not limitation, the removal of suppressors alone can act as a detectable signal without the need for label probes associated with the signal region such as when the mass or hydrodynamic radius of the first probe changes sufficiently to be measured by, for example, a change in electrophoretic mobility.

In any of the foregoing methods, the step of measuring a signal generated from the label probes associated with the signal regions of the first probe to determine the presence of the analyte in the sample can be any suitable time to measure the signal such as, by way of example but not limitation, from about 1 millisecond to about 10 minutes. By way of example, but not limitation, the step of measuring a signal generated from the label probes associated with the signal regions of the first probe to determine the presence of the analyte in the sample can be from about 1 millisecond to about 10 minutes, about 1 millisecond to about 5 minutes, about 1 millisecond to about 1 minute, about 1 millisecond to about 30 seconds, about 1 millisecond to about 15 seconds, about 1 millisecond to about 10 seconds, about 1 millisecond to about 10 seconds, about 1 millisecond to about 5 seconds, about 1 millisecond to about 1 second, about 1 millisecond to about 500 milliseconds, about 1 millisecond to about 400 milliseconds, about 1 millisecond to about 300 milliseconds, about 1 millisecond to about 200 milliseconds, about 1 millisecond to about 100 milliseconds, about 1 millisecond to about 50 milliseconds, about 1 millisecond to about 30 milliseconds, about 1 millisecond to about 20 milliseconds, about 1 millisecond to about 10 milliseconds, about 10 milliseconds to about 10 minutes, about 10 milliseconds to about 5 minutes, about 10 milliseconds to about 1 minute, about 10 milliseconds to about 30 seconds, about 10 milliseconds to about 15 seconds, about 10 milliseconds to about 10 seconds, about 10 milliseconds to about 10 seconds, about 10 milliseconds to about 5 seconds, about 10 milliseconds to about 1 second, about 10 milliseconds to about 500 milliseconds, about 10 milliseconds to about 400 milliseconds, about 10 milliseconds to about 300 milliseconds, about 10 milliseconds to about 200 milliseconds, about 10 milliseconds to about 100 milliseconds, about 10 milliseconds to about 50 milliseconds, about 10 milliseconds to about 30 milliseconds, about 10 milliseconds to about 20 milliseconds, about 20 milliseconds to about 10 minutes, about 20 milliseconds to about 5 minutes, about 20 milliseconds to about 1 minute, about 20 milliseconds to about 30 seconds, about 20 milliseconds to about 15 seconds, about 20 milliseconds to about 10 seconds, about 20 milliseconds to about 10 seconds, about 20 milliseconds to about 5 seconds, about 20 milliseconds to about 1 second, about 20 milliseconds to about 500 milliseconds, about 20 milliseconds to about 400 milliseconds, about 20 milliseconds to about 300 milliseconds, about 20 milliseconds to about 200 milliseconds, about 20 milliseconds to about 100 milliseconds, about 20 milliseconds to about 50 milliseconds, about 20 milliseconds to about 30 milliseconds, about 30 milliseconds to about 10 minutes, about 30 milliseconds to about 5 minutes, about 30 milliseconds to about 1 minute, about 30 milliseconds to about 30 seconds, about 30 milliseconds to about 15 seconds, about 30 milliseconds to about 10 seconds, about 30 milliseconds to about 10 seconds, about 30 milliseconds to about 5 seconds, about 30 milliseconds to about 1 second, about 30 milliseconds to about 500 milliseconds, about 30 milliseconds to about 400 milliseconds, about 30 milliseconds to about 300 milliseconds, about 30 milliseconds to about 200 milliseconds, about 30 milliseconds to about 100 milliseconds, about 30 milliseconds to about 50 milliseconds, about 50 milliseconds to about 10 minutes, about 50 milliseconds to about 5 minutes, about 50 milliseconds to about 1 minute, about 50 milliseconds to about 30 seconds, about 50 milliseconds to about 15 seconds, about 50 milliseconds to about 10 seconds, about 50 milliseconds to about 10 seconds, about 50 milliseconds to about 5 seconds, about 50 milliseconds to about 1 second, about 50 milliseconds to about 500 milliseconds, about 50 milliseconds to about 400 milliseconds, about 50 milliseconds to about 300 milliseconds, about 50 milliseconds to about 200 milliseconds, about 50 milliseconds to about 100 milliseconds, about 100 milliseconds to about 10 minutes, about 100 milliseconds to about 5 minutes, about 100 milliseconds to about 1 minute, about 100 milliseconds to about 30 seconds, about 100 milliseconds to about 15 seconds, about 100 milliseconds to about 10 seconds, about 100 milliseconds to about 10 seconds, about 100 milliseconds to about 5 seconds, about 100 milliseconds to about 1 second, about 100 milliseconds to about 500 milliseconds, about 100 milliseconds to about 400 milliseconds, about 100 milliseconds to about 300 milliseconds, about 100 milliseconds to about 200 milliseconds, about 200 milliseconds to about 10 minutes, about 200 milliseconds to about 5 minutes, about 200 milliseconds to about 1 minute, about 200 milliseconds to about 30 seconds, about 200 milliseconds to about 15 seconds, about 200 milliseconds to about 10 seconds, about 200 milliseconds to about 10 seconds, about 200 milliseconds to about 5 seconds, about 200 milliseconds to about 1 second, about 200 milliseconds to about 500 milliseconds, about 200 milliseconds to about 400 milliseconds, about 200 milliseconds to about 300 milliseconds, about 300 milliseconds to about 10 minutes, about 300 milliseconds to about 5 minutes, about 300 milliseconds to about 1 minute, about 300 milliseconds to about 30 seconds, about 300 milliseconds to about 15 seconds, about 300 milliseconds to about 10 seconds, about 300 milliseconds to about 10 seconds, about 300 milliseconds to about 5 seconds, about 300 milliseconds to about 1 second, about 300 milliseconds to about 500 milliseconds, about 300 milliseconds to about 400 milliseconds, about 400 milliseconds to about 10 minutes, about 400 milliseconds to about 5 minutes, about 400 milliseconds to about 1 minute, about 400 milliseconds to about 30 seconds, about 400 milliseconds to about 15 seconds, about 400 milliseconds to about 10 seconds, about 400 milliseconds to about 10 seconds, about 400 milliseconds to about 5 seconds, about 400 milliseconds to about 1 second, about 400 milliseconds to about 500 milliseconds, about 500 milliseconds to about 10 minutes, about 500 milliseconds to about 5 minutes, about 500 milliseconds to about 1 minute, about 500 milliseconds to about 30 seconds, about 500 milliseconds to about 15 seconds, about 500 milliseconds to about 10 seconds, about 500 milliseconds to about 10 seconds, about 500 milliseconds to about 5 seconds, about 500 milliseconds to about 1 second, about 1 second to about 10 minutes, about 1 second to about 5 minutes, about 1 second to about 1 minute, about 1 second to about 30 seconds, about 1 second to about 15 seconds, about 1 second to about 10 seconds, about 1 second to about 10 seconds, about 1 second to about 5 seconds, about 5 seconds to about 10 minutes, about 5 seconds to about 5 minutes, about 5 seconds to about 1 minute, about 5 seconds to about 30 seconds, about 5 seconds to about 15 seconds, about 5 seconds to about 10 seconds, about 5 seconds to about 10 seconds, about 10 seconds to about 10 minutes, about 10 seconds to about 5 minutes, about 10 seconds to about 1 minute, about 10 seconds to about 30 seconds, about 10 seconds to about 15 seconds, about 15 seconds to about 10 minutes, about 15 seconds to about 5 minutes, about 15 seconds to about 1 minute, about 15 seconds to about 30 seconds, about 30 seconds to about 10 minutes, about 30 seconds to about 5 minutes, about 30 seconds to about 1 minute, about 1 minute to about 10 minutes, about 1 minute to about 5 minutes, about 5 minutes to about 10 minutes, or about 1 millisecond, 10 milliseconds, 20 milliseconds, 30 milliseconds, 50 milliseconds, 100 milliseconds, 200 milliseconds, 300 milliseconds, 400 milliseconds, 500 milliseconds, 1 second, 5 seconds, 10 seconds, 15 seconds, 30 second, 1 minute, 5 minutes, or 10 minutes.

In any of the foregoing embodiments, the amount of analyte can be at least 1 copy (1.66×10⁻²⁴ moles). In any of the foregoing embodiments, the amount of the analyte can be between about 1 copy (1.66×10⁻²⁴ moles) and about 6×10²⁰ copies (1 millimole). By way of example, but not limitation, the amount of analyte can be between about 1000 and 100,000,000 copies. In any of the foregoing embodiments, the amount of analyte can be between about 1 attomole and about 100 picomoles. By way of example, but not limitation, the amount of analyte can be between about 1 attomole and about 1 femtomole. In any of the foregoing embodiments, the concentration of analyte in the sample can be at least one zeptomolar, at least one attomolar, or at least one femtomolar. By way of example, but not limitation, the concentration of analyte in the sample can be between about 1 attomolar and about 1 nanomolar. In any of the foregoing embodiments, the concentration of the analyte in the sample can be at least 1 femtogram per milliliter, at least 1 picogram per milliliter, or at least 1 nanogram per milliliter. By way of example, but not limitation, the concentration of analyte in the sample can be between about 1 femtogram per milliliter and about 100 nanograms per milliliter. By way of example, but not limitation, the concentration of analyte in the sample can be between about 10 femtograms per milliliter and about 1 nanogram per milliliter.

In any of the foregoing methods, the conditions sufficient can be, for nucleic acid detection, a buffer ionic strength of at least 10 mM, preferably at least 100 mM, buffer pH of between about 5 and about 9, and temperature of about 4° C. to about 50° C. during all steps after the addition of the analyte to the first probe. If any essential components of the assay (analyte, probes, etc.) comprise RNA nucleotides without 2′-O-methyl, 2′-fluoro, or phosphorothioate modifications, it may be preferable to add ethylenediaminetetraacetic acid (EDTA) to a final concentration of at least 1 millimolar or at least 10 millimolar, or a concentration greater than the combined concentrations of all divalent and trivalent metal cations in the mixture. If RNases are known to be present in the mixture and are not part of the intended mechanism of the assay (e.g., suppressor degradation), it may be advantageous to add an RNase inhibitor at a sufficient concentration to inhibit unwanted degradation of the RNA components in the assay. In any of the foregoing methods, the conditions can be, for proteins, a buffer ionic strength of about 10 mM to about 500 mM, buffer pH of between about 5 to about 9, and a temperature of about 4° C. to about 50° C. after addition of the analyte to the first probe. In any of the foregoing methods, the step of binding the analyte to the first analyte binding region of the first probe can be allowed to proceed for about 10 minutes to about 24 hours. In any of the foregoing methods, the step of incubating the second probe with the mixture containing the first probe and analyte can be allowed to proceed for about 1 minute to about 24 hours.

It should be understood that in any of the foregoing methods, the components, e.g. the first probes and second probes, can have the features and characteristics from any of the embodiments of the present disclosure. It should also be understood that although the present disclosure does not describe a separate method for each of the combinations of various configurations of the components parts of the systems and kits described herein (first probe, analyte binding region, signal region, suppressors, second probes, label probes, multivalent capture probe, capture probe, etc) one of ordinary skill in the art would be able to modify the methods described herein with the various alternative component configurations and embodiments described herein without undue experimentation.

### EXAMPLES

### Example 1: Detection of a DNA Sequence

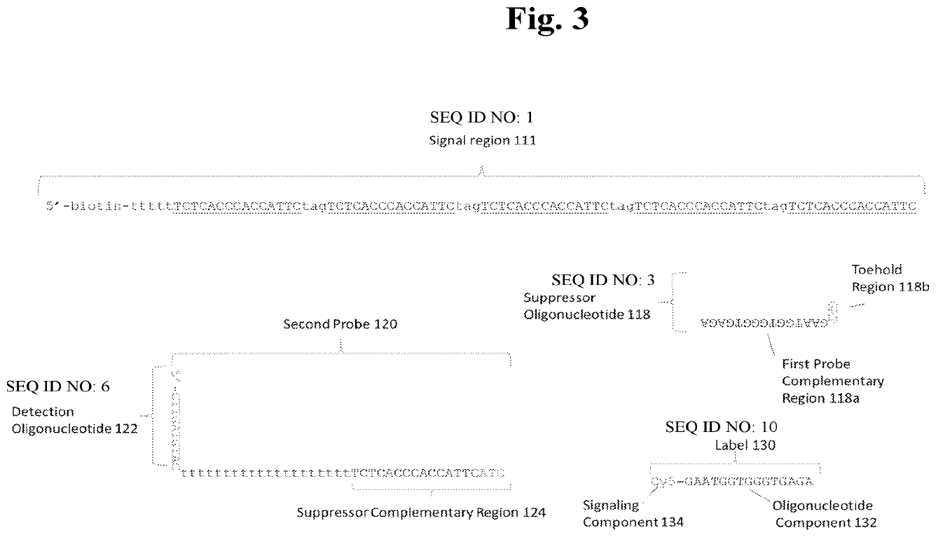

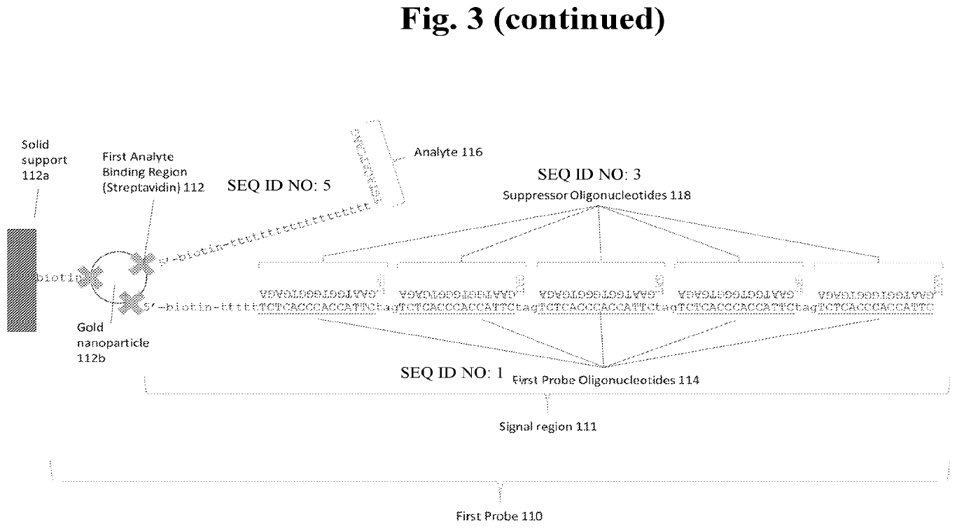

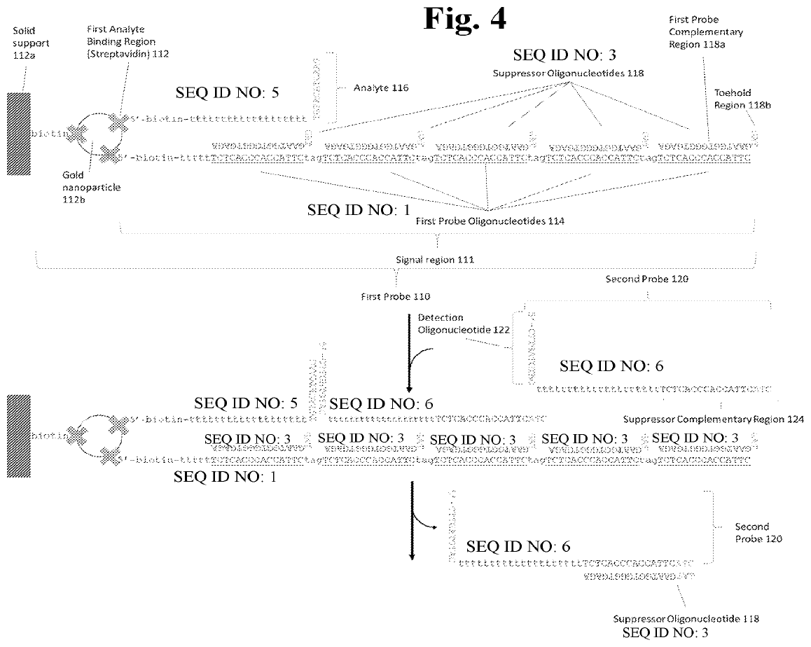

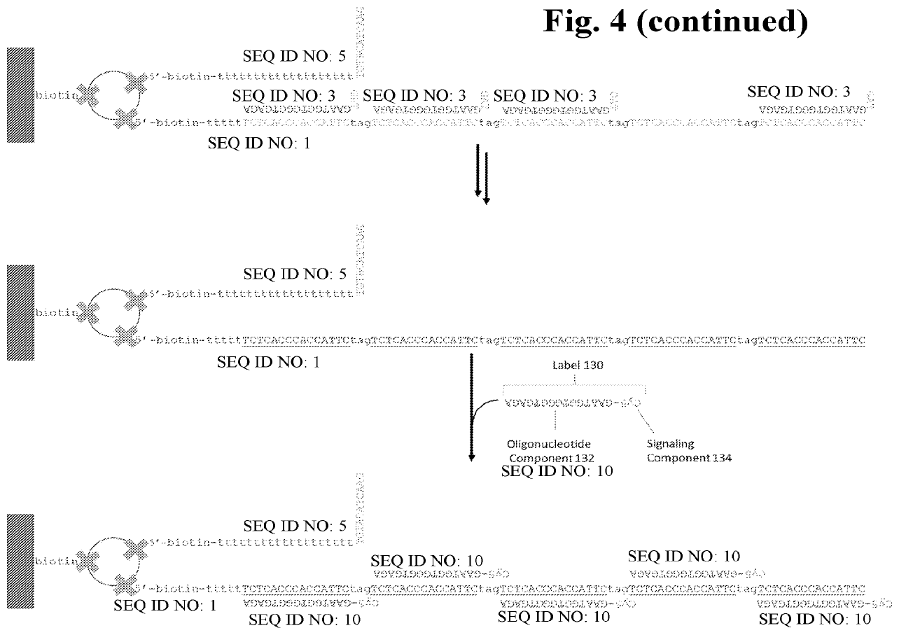

This example and its related data are depicted in FIGS. 3 to 6.

Streptavidin-coated gold nanoparticles (20 nm nominal diameter, 112b) were bound to a biotin-coated microscope coverslip (solid support, 112a). A biotin-labeled DNA oligonucleotide 111 (signal region) (SEQ ID NO: 1) which included 5 equivalent binding sites (first probe oligonucleotides, 114 (SEQ ID NO: 2)) for a complementary suppressor DNA oligonucleotide 118 (SEQ ID NO: 3) was combined with a 10-fold molar excess of the complementary suppressor DNA oligonucleotides 118 (SEQ ID NO: 3) in 1X phosphate buffered saline (PBS) buffer and thermally annealed by heating to 70° C. and then cooling to room temperature (approximately 22° C.) for 5 minutes. Each suppressor DNA oligonucleotide 118 included a first probe complementary region 118a (SEQ ID NO: 4) and a 5′ toehold region comprising GAT 118b. A biotin-labeled analyte DNA oligonucleotide 116 (SEQ ID NO: 5) was combined with a 100-fold molar excess of the mixture of biotin-labeled DNA oligonucleotide 111 (SEQ ID NO: 1) and complementary suppressor DNA oligonucleotides 118 (SEQ ID NO: 3), then added to the coverslip surface containing the streptavidin-coated gold nanoparticles to allow the biotin-labeled analyte DNA oligonucleotide 116 (SEQ ID NO: 5) and the biotin-labeled DNA oligonucleotide 111/suppressor DNA oligonucleotide 118 complex to bind to the gold nanoparticles to give the first probe 110 which included the first analyte binding region 112, in this case streptavidin. It was estimated that only about 10 biotinylated oligonucleotides can bind to each gold nanoparticle under these conditions, so approximately 10% of the gold nanoparticles are expected to contain one or more copies of the biotin-labeled analyte DNA oligonucleotide 116, and the majority are expected to contain no copies of the biotin-labeled analyte DNA oligonucleotide 116; however, all nanoparticles are expected to contain about 9-10 copies of the complex of the biotin-labeled DNA oligonucleotide 111 and suppressor DNA oligonucleotide 118 . . . . In a corresponding negative control, the biotin-labeled analyte DNA oligonucleotide 116 was omitted, and only the biotin-labeled DNA oligonucleotide 111/suppressor DNA oligonucleotide 118 complex was incubated with the gold nanoparticles. After 20 minutes, the excess oligonucleotide solution was removed, and the surface was washed twice with 4×PBS buffer.

Next, a second probe 120 (SEQ ID NO: 6) was added to the coverslip surface at a concentration of 25 nanomolar (nM) in 4×PBS buffer. The second probe 120 included a detection oligonucleotide portion 122 (SEQ ID NO: 7) that forms 10 complementary base pairs with the biotin-labeled analyte DNA oligonucleotide 116, as well as an inert poly(deoxythymidine) linker (SEQ ID NO: 8) and a suppressor complementary region 124 (SEQ ID NO: 9) that can remove a suppressor oligonucleotide 118 from the biotin-labeled DNA oligonucleotide 111 by toehold-mediated strand displacement (TMSD). Since each suppressor oligonucleotide 118 contains a toehold sequence of only 3 nucleotides when the suppressor oligonucleotide 118 is bound to the first probe 110, the removal of the suppressor oligonucleotide 118 is inefficient in the presence of 25 nM of the second probe 120 unless the biotin-labeled analyte DNA oligonucleotide 116 is also present (e.g., it is not present in the negative control experiment). However, when the second probe 120 is bound to the biotin-labeled analyte DNA oligonucleotide 116, the 3 nucleotide toehold is sufficient for the second probe 120 to rapidly remove exactly one copy of the suppressor oligonucleotide 118 from a proximal first probe 110 by TMSD. Furthermore, since the second probe 120 is only complementary for a length of 10 base pairs with the biotin-labeled analyte DNA oligonucleotide 116, the second probe 120 dissociates rapidly from the biotin-labeled analyte DNA oligonucleotide 116 at the incubation temperature of 25° C., permitting another copy of the second probe 120 to bind to the biotin-labeled analyte DNA oligonucleotide, remove another copy of the suppressor oligonucleotide 118 by TMSD, and dissociate. After many cycles of such binding, many copies of the suppressor oligonucleotide 118 have been removed from one or more signal regions 111 attached to the same nanoparticle (note that the complex of the nanoparticle and one or more copies of the biotin-labeled DNA oligonucleotide 111 can be itself considered the first probe 110).

After an incubation period of 30-130 minutes (in this case, 45 minutes), the solution of the second probe 120 was removed, and a new solution containing 10 nM of a fluorescently-labeled label oligonucleotide 130 (SEQ ID NO: 10) was added. The label oligonucleotide 130 included a signaling component 134, in this case the Cy5 fluorophore, and an oligonucleotide component 132 with a DNA sequence complementary to the binding sites for the suppressor oligonucleotides 118, and therefore binds to those sites where a copy of the suppressor oligonucleotide 118 has been removed. After a 10-minute incubation period at room temperature, the solution of the fluorescently labeled label oligonucleotide 130 was removed, and a solution comprising an oxygen scavenger system (protocatechuate dioxygenase, 3,4-dihydroxybenzoic acid, and Trolox) in 4×PBS was added. The fluorescence intensity of the surface-bound nanoparticles complexes was measured by objective-type total internal reflection fluorescence (TIRF) microscopy with excitation from a 640 nm continuous wave laser and detection using a scientific complementary metal oxide semiconductor (sCMOS) camera with an exposure time of about 500 milliseconds.

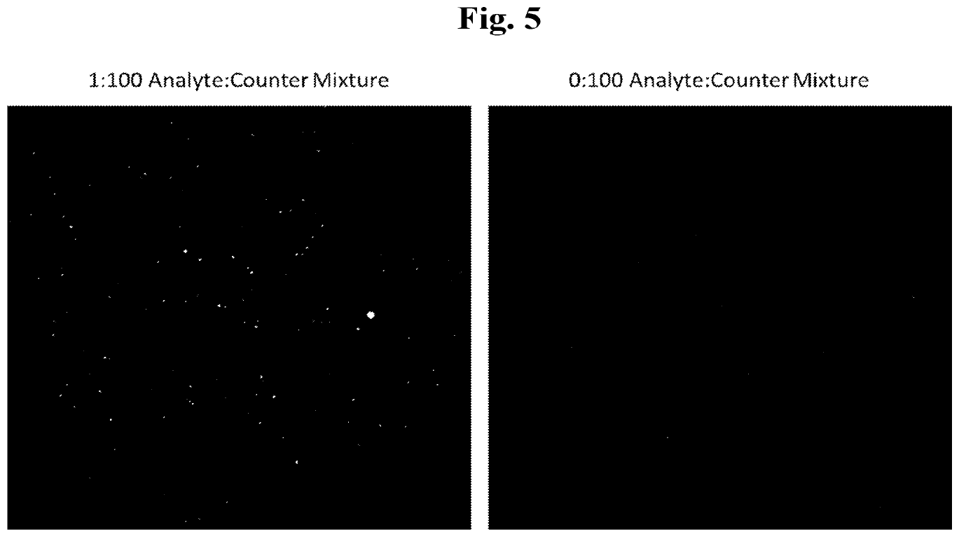

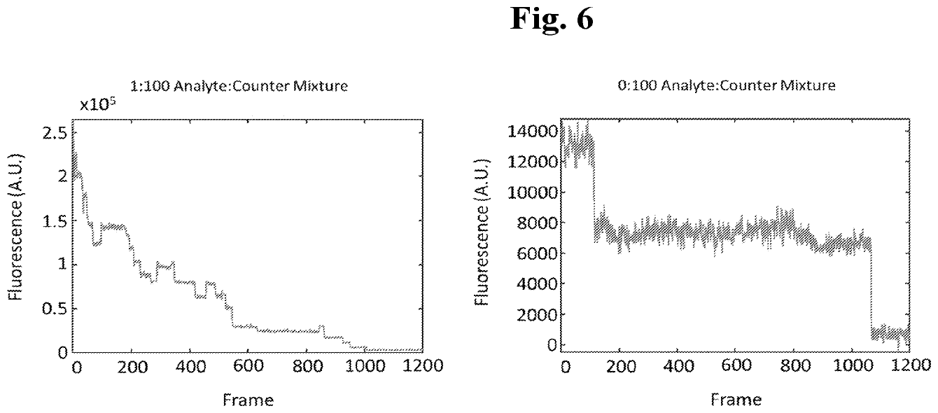

As shown in FIG. 5, the measurement revealed many bright puncta for the experiment in the presence of the biotin-labeled analyte DNA oligonucleotide 116 (“1:100 Analyte: Counter Mixture”), but a lack of equally bright puncta in the experiment performed in the absence of the biotin-labeled analyte DNA oligonucleotide 116 (“0:100 Analyte: Counter Mixture”) indicating that the removal of many copies of the suppressor oligonucleotide 118 from the biotinylated signal region oligonucleotides 111 bound to a single gold nanoparticle as part of a single first probe 110 is more efficient in the presence of the biotin-labeled analyte DNA oligonucleotide 116 than in its absence. It should be noted that the contrast of the images in FIG. 5 was adjusted to make dimmer spots invisible; however, both images are adjusted to the same contrast range and were collected under identical illumination and detection conditions. Furthermore, prolonged exposure of the sample to high-intensity laser excitation light permitted analysis of the number of photobleaching steps per particle as shown in FIG. 6; particles exhibiting >10 photobleaching steps (indicating the presence of >10 copies of the fluorescently-labeled label oligonucleotide 130, and therefore the removal of >10 copies of the suppressor oligonucleotide 118) are common in the experiment where the biotin-labeled analyte DNA oligonucleotide 116 was present (“1:100 Analyte: Counter Mixture”) but rare or absent in the negative control experiment where the biotin-labeled analyte DNA oligonucleotide 116 was omitted (“0:100 Analyte: Counter Mixture”). In the absence of the biotin-labeled analyte DNA oligonucleotide 116, the majority of particles exhibited only 1-5 photobleaching steps and none exhibited >10 photobleaching steps.

Taken together, these results indicate that the interaction of the second probe 120 and the biotin-labeled analyte DNA oligonucleotide 116 results in the removal of several copies of the suppressor oligonucleotide 118 from each copy of the first probe 110 with higher efficiency than is possible in the absence of the biotin-labeled analyte DNA oligonucleotide 116 and, therefore, the presence of bright puncta can be used to infer the presence of the biotin-labeled analyte DNA oligonucleotide 116 in the mixture.

### Example 2: Assessment of the Effect of Second Probe Incubation Time

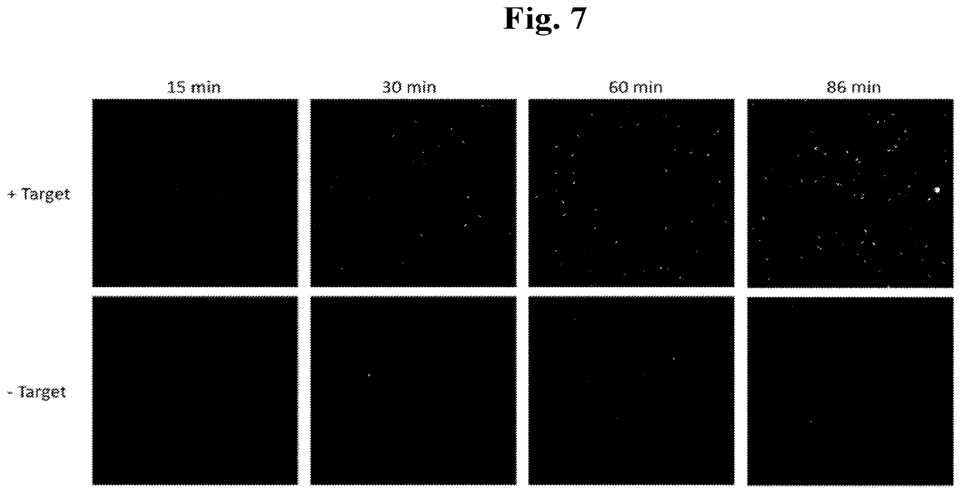

In a separate experiment, the methodology of Example 1 was followed except that the second probe 120 was allowed to incubate with the loaded nanoparticles for varying amounts of time—15, 30, 60, or 86 minutes—before removing the second probe 120 and proceeding with the addition of the fluorescently-labeled label oligonucleotide 130. As shown in FIG. 7, which provides TIRF microscopy images for the various time points with analyte (+ Target) or without analyte (− Target), the number and fluorescence intensity of puncta increased with longer incubation time, consistent with the expectation that longer incubation times will allow for more second probe 120 binding events to the same analyte-loaded particle, and thus remove more suppressors and generate more intense signal from individual first probes.

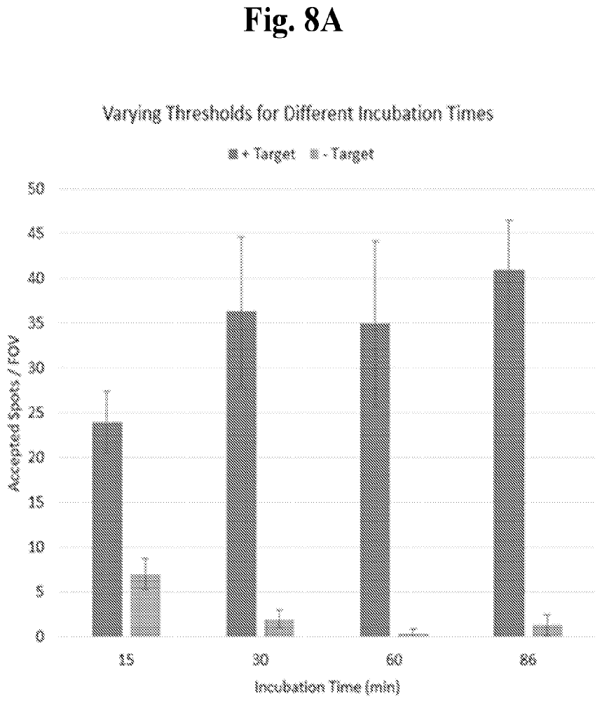

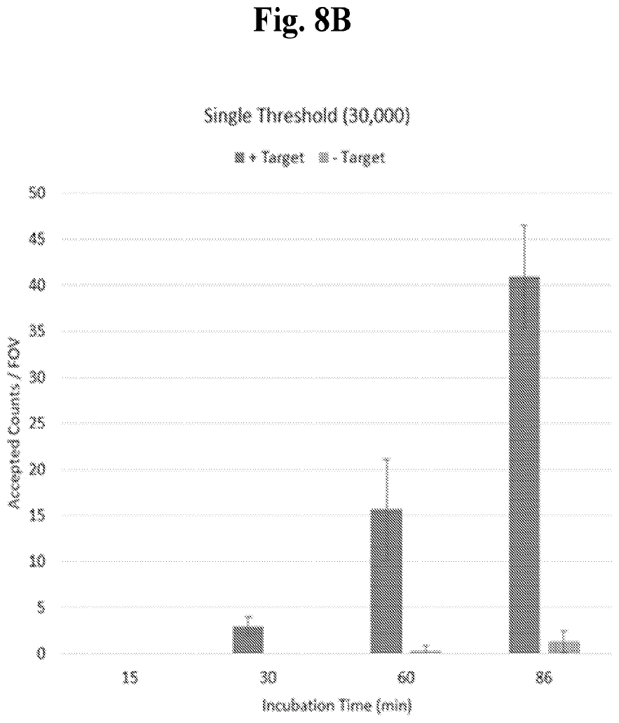

FIGS. 8A-8B depict quantification of the results in FIG. 7 either with varying thresholds for different incubation times (FIG. 8A) or using a single threshold of 30,000 arbitrary units (FIG. 8B). Accepted spots (or accepted counts) per FOV include those puncta satisfying the minimum intensity thresholds. In the case of varying thresholds (FIG. 8A) in this experiment, thresholds were manually chosen to maximize the difference between the number of accepted sports in the positive control (+ Target) measurement and the number of accepted spots in the negative control (− Target) measurement for each experimental condition (i.e., for each incubation time) while maintaining fewer than 10 accepted counts per field-of-view (FOV) in the negative control measurement. In the case of analysis with a single threshold (FIG. 8B), the threshold chosen for the longest incubation time (86 min) was applied to all of the experimental conditions. If different thresholds are used for different incubation times, similar discrimination between experiments with and without analyte (+ Target and − Target, respectively) can be achieved for experiments with 30, 60 and 86 minute incubations. If a single intensity threshold of 30,000 arbitrary units is used for all incubation times, it is clear that the number of very bright puncta continues to increase throughout the time course.

### Example 3: Assessment of the Effect of Varying Analyte Concentration

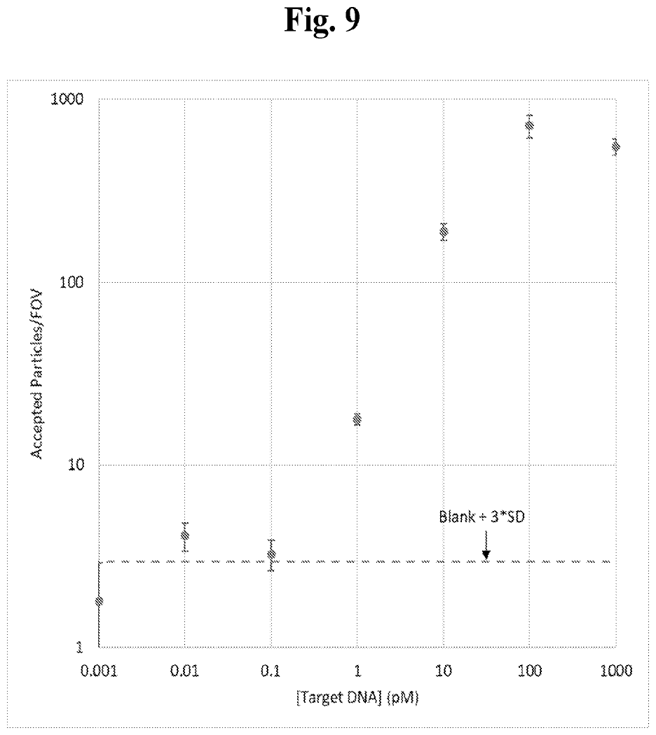

In a separate experiment, a target DNA oligonucleotide (analyte) similar to that shown in FIG. 3 (the sequence of the analyte for this experiment was 5′-biotin-TTTTTTTTTTTTTTTTTTATGTACATCAAG (SEQ ID NO: 17)) was introduced at varying concentrations (10 nM, 1 nM, 100 pM, 10 pM, 1 pM, 100 femtomolar (fM), 10 fM, 1 fM, or 0 fM) to a microscope coverslip coated with streptavidin-modified gold nanoparticles and incubated for 30 minutes to allow capture of the biotinylated target DNA oligonucleotide (analyte). The signal region bound to suppressor oligonucleotides was then introduced at 50 nM and incubated for 20 minutes to allow it to bind to the same nanoparticles. The experiment was then performed as described in Example 1, but using a 60-minute incubation in the presence of the second probe at 22.5° C. After binding of the fluorescently-labeled label oligonucleotide to exposed sites on the signal region, bright puncta were again observed, with the number of bright puncta dependent on the concentration of target oligonucleotide (analyte) that had been incubated with the particles. Quantification of the number of particles with fluorescent intensity between minimum and maximum threshold of 25,000 and 100,000 arbitrary units, respectively, is shown in FIG. 9, and indicates that the concentration of the target (analyte) can be quantified using this approach.

All concentrations of 10 fM and higher yielded counts of bright puncta per field of view satisfying the intensity thresholds (accepted counts) at least 3 standard deviations higher than the mean signal of the negative control experiment, suggesting a limit of detection of approximately 10 fM. This only required only 12.5 seconds of measurement time (0.5 seconds per field of view×25 fields of view).

### Example 4: Detection of a Protein

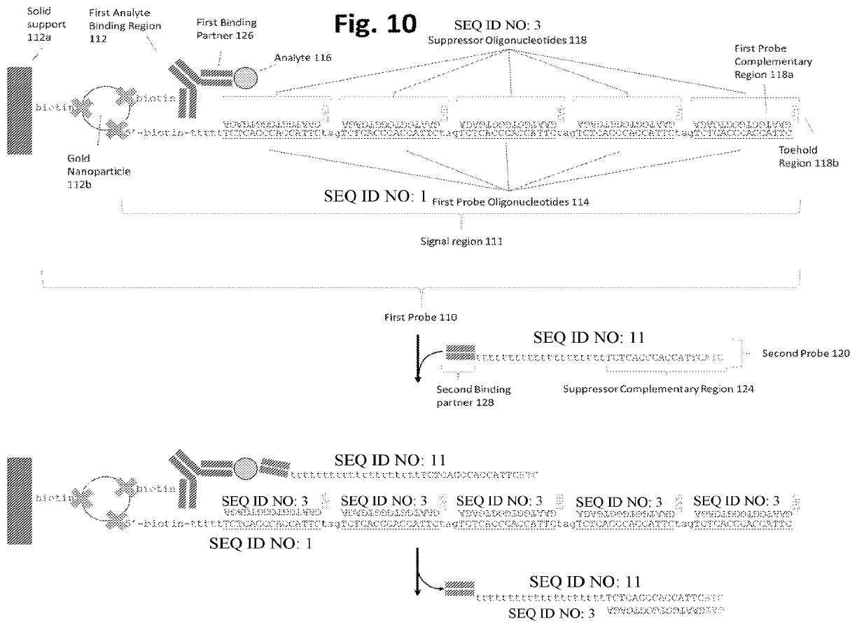

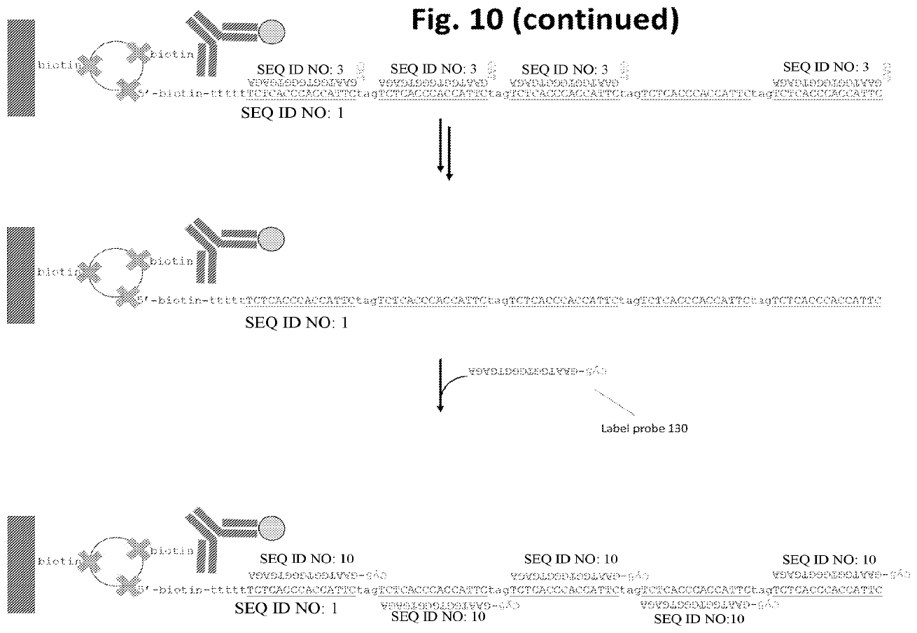

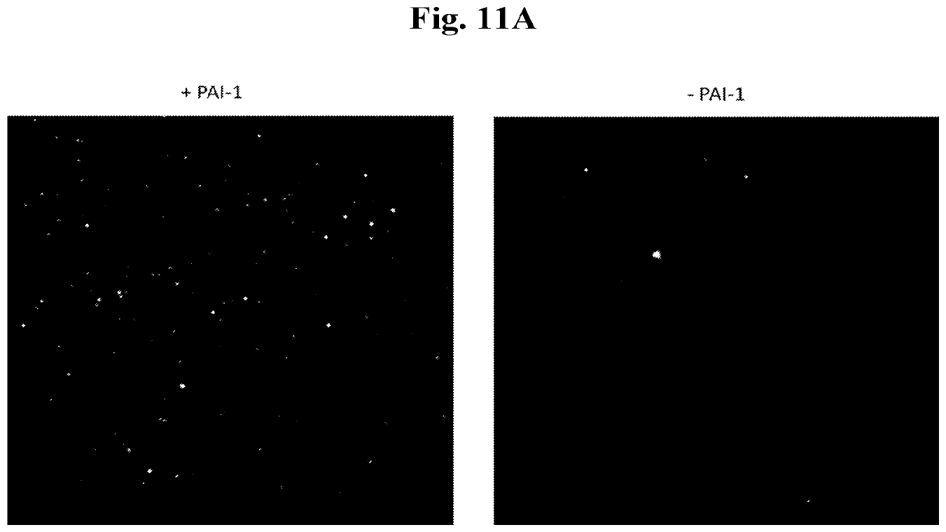

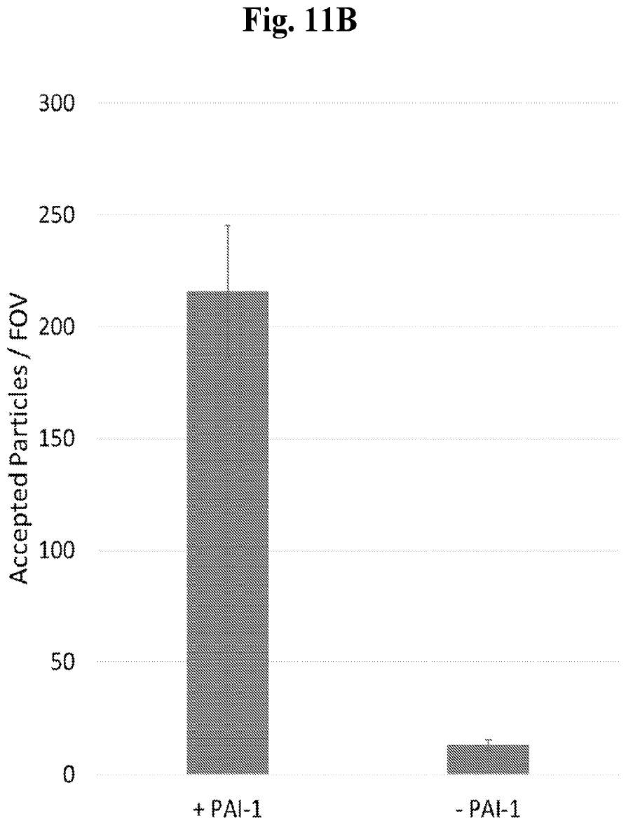

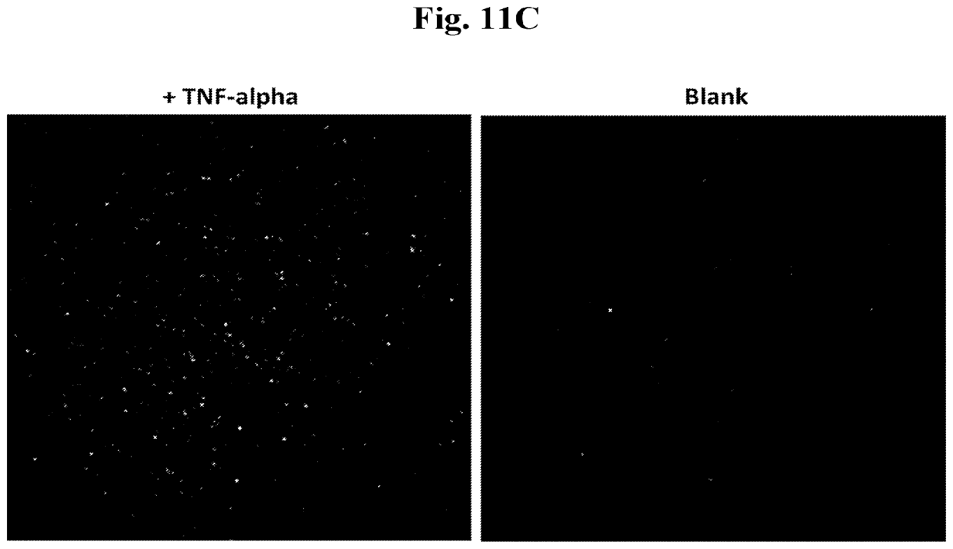

This example and its related data are depicted in FIGS. 10 to 11C.

This experiment was conducted to demonstrate that the same approaches used for DNA oligonucleotide detection can be adapted for the detection of other types of biomolecular analytes, such as proteins.

The system shown in FIG. 10 was demonstrated in an experiment designed to detect the protein analyte PAI-1 (plasminogen activator inhibitor-1) (analyte, 116). In this experiment, streptavidin-coated gold nanoparticles (20 nm nominal diameter) were bound to a biotin-coated microscope coverslip. Next, a biotin-labeled DNA oligonucleotide (signal region, 111) (SEQ ID NO: 1) comprising 5 equivalent binding sites (first probe oligonucleotides 114 (SEQ ID NO: 2)) for a complementary suppressor DNA oligonucleotide 118 (SEQ ID NO: 3) which includes a first probe complementary region 118a (SEQ ID NO: 4) and a toehold region 118b having the sequence GAT was combined with a 10-fold molar excess of suppressor oligonucleotides 118 in 1×PBS buffer and thermally annealed by heating to 70° C. and then cooled to room temperature for 5 minutes. A biotin-labeled capture antibody (first binding partner, 126) against PAI-1 was combined with a 4-fold molar excess of the biotin-labeled DNA oligonucleotide 111/suppressor oligonucleotide 118 mixture in 1×PBS, then added to the slide surface 112a containing the streptavidin-coated gold nanoparticles 112b to allow the capture antibody and biotin-labeled DNA oligonucleotide 111/suppressor oligonucleotide 118 complex to bind to the gold nanoparticles 112b for 20 minutes at room temperature to form the first probe 110 which includes the first analyte binding region 112. The solution was removed, and the surface washed four times with 1×PBS.

Next, a mixture containing the analyte PAI-1 116 at a concentration of 4 ng/ml (prepared from the Bio-Plex Pro Human Diabetes Standards, P/N 171D70001, Bio-Rad Laboratories) in 10 mg/mL bovine serum albumin (BSA) and 1×PBS was added to the surface. In a corresponding negative control, the PAI-1 analyte standard mixture was omitted and a solution of 10 mg/mL BSA in 1×PBS was instead added. After 30 minutes to allow for capture of any analyte present, the solution was removed, and the surface washed twice with 1×PBS.

Next a Fab (antibody) fragment against PAI-1 (second binding partner, 128) that had been covalently conjugated to a second probe oligonucleotide using dibenzocyclooctyne/azide click chemistry—i.e., a Fab-second probe oligonucleotide conjugate—to yield a second probe 120 was added to the surface at a concentration of 24 nM in 1× Tris-buffered saline (TBS) containing 0.1% Tween 20. When the second probe 120 is bound to the analyte 116, the 3-nucleotide toehold of each second probe 120 is sufficient for the second probe to rapidly remove one copy of the suppressor oligonucleotide 118 from a proximal first probe 110 by TMSD, since the second probe 120 is present at a locally high concentration in the vicinity of the first probe 110. Furthermore, since the Fab binds only transiently with the analyte 116, the second probe 120 dissociates rapidly from PAI-1 at the incubation temperature of 25° C., permitting another copy of the second probe 120 to bind to the same copy of PAI-1, remove another copy of the suppressor oligonucleotide 118 by TMSD, and dissociate. After many cycles of such binding, many copies of the suppressor oligonucleotide have been removed from a signal region comprising one or more copies of oligonucleotide 111 attached to the same nanoparticle (note that the complex of the nanoparticle and biotin-labeled DNA oligonucleotide 111 can be itself considered the first probe 110).

After an incubation period of 45 minutes, the solution of the second probe 120 was removed, and a new solution containing 10 nM of a fluorescently-labeled label probe 130 was added. After a 10 minute incubation period at room temperature, the solution of the fluorescently-labeled label probe was removed, and a solution comprising an oxygen scavenger system (protocatechuate dioxygenase, 3,4-dihydroxybenzoic acid, and Trolox) in 4×PBS was added. The fluorescence intensity of the surface-bound nanoparticle complexes was measured by objective-type total internal reflection fluorescence (TIRF) microscopy with excitation from a 640 nm continuous wave laser and detection using a scientific metal oxide semiconductor (sCMOS) camera.

The measurement revealed many bright puncta for the experiment in the presence of PAI-1, but few equally bright puncta in the experiment performed in the corresponding negative control (see FIGS. 11A-11B), indicating that the removal of many copies of the suppressor oligonucleotide 118 from the first probes 110 is more efficient in the presence of PAI-1 than its absence, and that the presence and concentration of PAI-1 can be inferred from the number of bright puncta in the acquired images (i.e., the number of nanoparticles bearing sufficient labels to make them brighter than the vast majority of nanoparticles in a corresponding negative control). FIG. 11B provides quantification of results from 25 fields of view and shows that the number of particles satisfying minimum and maximum intensity thresholds is much greater in the presence of analyte PAI-1 than in its absence.

In addition to PAI-1, the detection of another protein, tumor necrosis factor alpha (TNF-alpha) was demonstrated using the approach shown in FIG. 10. In this case, the second probe include an oligonucleotide conjugated to a detection antibody using trans-cyclooctene/tetrazine click chemistry, and the first analyte binding region (capture antibody) and second binding partner (detection antibody) were specific to TNF-alpha. However, the oligonucleotide sequences, gold nanoparticles, and assay protocol were identical to those used for the detection of PAI-1. As with PAI-1, after incubation with the second probe followed by a fluorescent label oligonucleotide, TIRF microscopy revealed a larger number of bright puncta in the presence of the analyte TNF-alpha (left) than in its absence (right) (FIG. 11C), indicating the ability to detect the antigen TNF-alpha using this approach.

### Example 5: Additional System and Method Designs

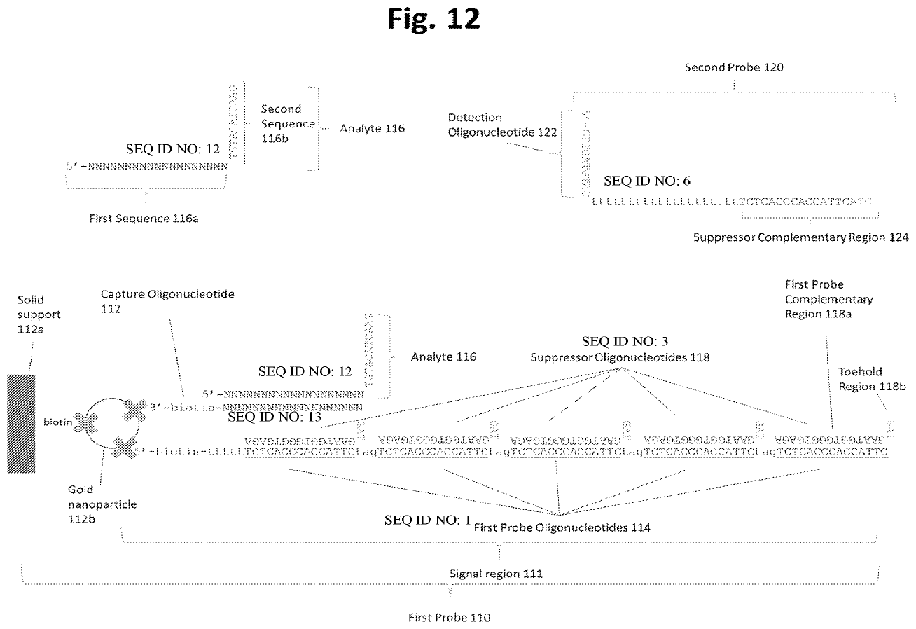

Another exemplary system of the present disclosure is depicted in FIG. 12.

Instead of detecting a biotin-modified nucleic acid analyte, a non-biotinylated nucleic acid analyte can be detected by instead employing a biotinylated capture oligonucleotide bearing a sequence that is complementary to a portion of the sequence of the analyte, and which stably captures the analyte near the signal region for the assay.

As shown in FIG. 12, a biotinylated signal region 111 which includes first probe oligonucleotides 114 can be linked to a streptavidin-coated gold nanoparticle 112b which is linked to a biotinylated solid support 112a. A biotinylated capture oligonucleotide which includes a first analyte binding region 112 (capture oligonucleotide) can be linked to the same gold nanoparticle via a biotin-streptavidin interaction to form the first probe 110. The capture oligonucleotide can include any sequence that is complementary to at least a first sequence 116a of an analyte which can have a second sequence 116b. The suppressor oligonucleotides can include a first probe complementary region 118a and a toehold region 118b. A second probe 120 which includes a detection oligonucleotide 122 and a suppressor complementary region 124 can be added which will hybridize with the second sequence 116b of the analyte which has hybridized by its first sequence 116a to the capture oligonucleotide 112. The suppressor complementary region 124 can then remove a suppressor oligonucleotide 118 by TMSD. Once the one or more suppressors are removed, the label 130, which includes a signaling component 134 and an oligonucleotide component 132, which is complementary to the first probe oligonucleotides, can be added and associate with the signal region 111 where the one or more suppressors have been removed to produce a signal. It should be understood that the nucleotides designated N can be any nucleotides and should be sufficiently complementary between the capture oligonucleotide and the analyte for stable capture at the temperature and buffer conditions used. It should also be understood that the second sequence 116b and detection oligonucleotide 122 can comprise any naturally occurring or synthetic nucleic acid sequences, provided that detection oligonucleotide 122 is complementary to second sequence 116b of the analyte and the dissociation of detection oligonucleotide 122 from second sequence 116b is sufficiently rapid to permit the binding and dissociation of multiple second probes 120 to and from the same copy of analyte 116 before the analyte 116 dissociates from capture oligonucleotide 112.

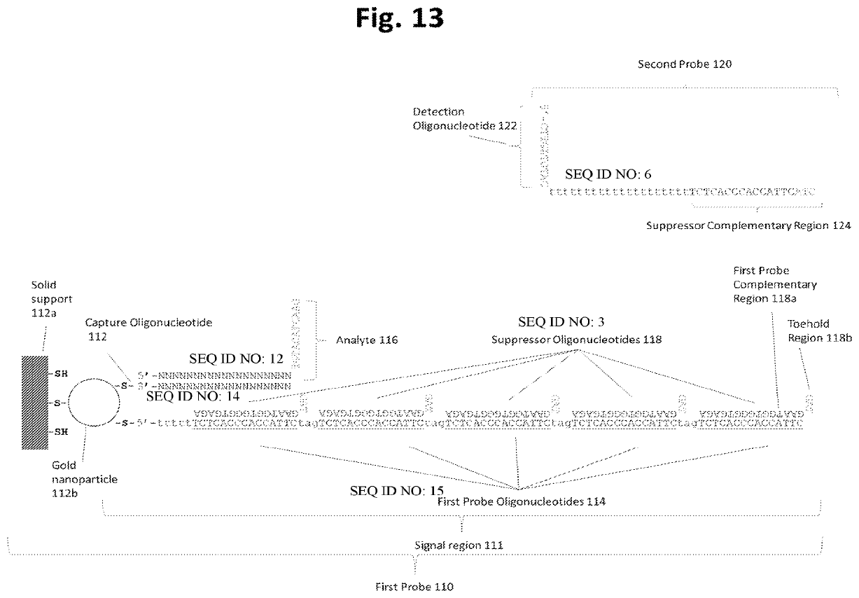

FIG. 13 depicts a similar system where the streptavidin-biotin interactions between the solid support and gold nanoparticle and between the gold nanoparticle and both the capture oligonucleotide and signal region are replaced by gold-thiol interactions. It should be understood that many other covalent and non-covalent coupling chemistries can be substituted in place of the streptavidin-biotin or gold-thiol interactions, with no significant impact on the function of other components of the system.

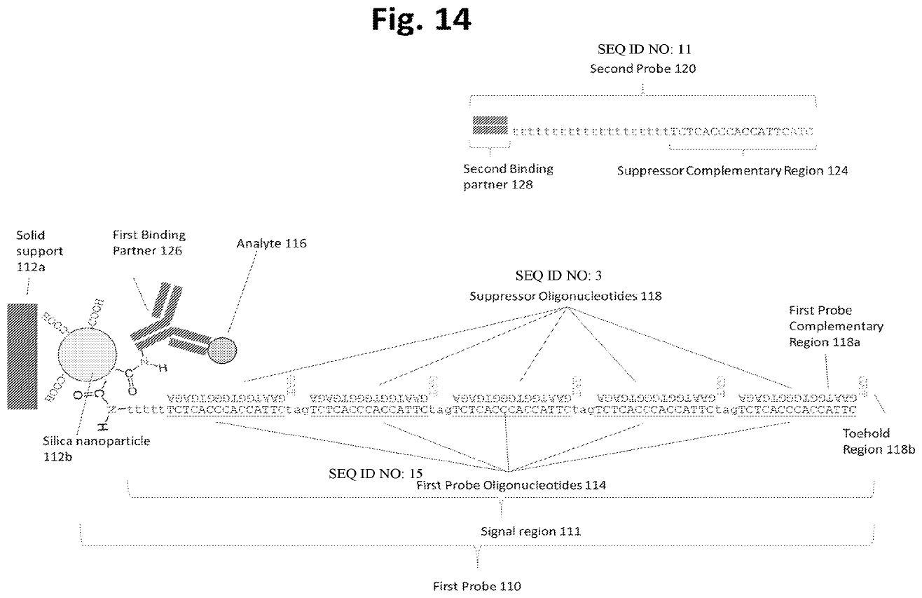

As shown in FIG. 14, an exemplary protein assay system which includes a first probe 110 and a second probe 120 can include a carboxylic acid-modified silica nanoparticle 112b that is conjugated to a capture antibody (first analyte binding region, 112) and an amine modified signal region 111 which includes first probe oligonucleotides 114 via EDC-NHS coupling. In this example, the nanoparticle can be bound to a surface (solid support, 112a) via noncovalent interactions, but could also be coupled to the assay surface covalently (e.g., via EDC-NHS coupling to an amine-modified surface). As shown, in the presence of the analyte 116 the second probe 120 which includes a suppressor complementary region 124 and a second binding partner 128, such as an antibody or fragment thereof, can bind to the analyte and through the suppressor complementary region remove one or more of the suppressor oligonucleotides 118 which include a first probe complementary region 118a and a toehold region 118b. Subsequent detection can be performed as described herein.

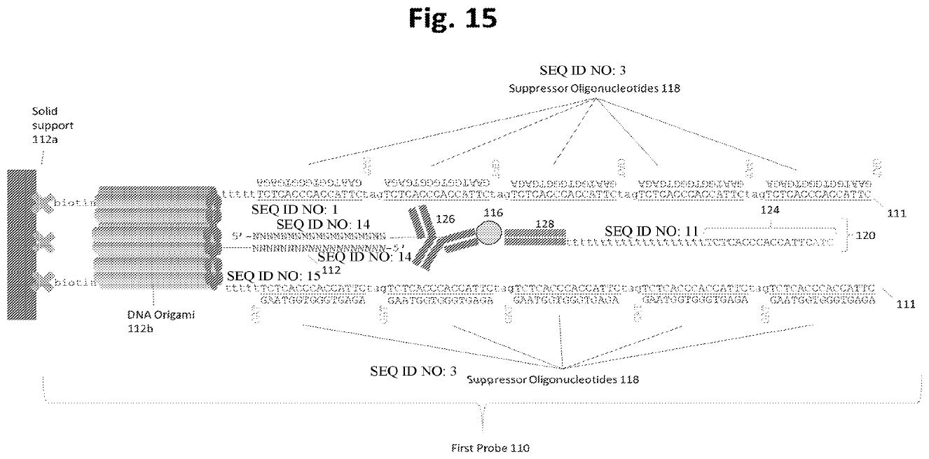

As shown in FIG. 15, an exemplary protein assay system using DNA origami to position the first analyte binding region and the signal regions in close proximity to one another, and with control of stoichiometry between the two as well as projection of other function components away from the assay surface and into solution. The projection of components into solution is facilitated by the ability to site-specifically position one, two, or more surface-tethering ligands or functional groups (biotin, in this example) on the DNA origami to ensure that the structure is anchored in a particular orientation relative to the assay surface. In this system, the capture antibody 126 (first binding partner) and signal region 111 are bound to a DNA origami structure 112b based on a honeycomb lattice architecture; the DNA origami 112b is in turn bound to a surface 112a via the streptavidin-biotin interaction. One skilled in the art of DNA nanotechnology will appreciate that it is within ordinary skill in the art to site-specifically decorate a DNA origami with signal region oligonucleotides 111, DNA-antibody conjugates, and biotin groups or other affinity tags, in the manner shown in FIG. 15, as well as to realize many other arrangements of the signal region oligonucleotides and capture probes and/or analyte, and to generate DNA origami with a wide variety of other two- or three-dimensional structures. Unlike the nanoparticle architectures described above, the relative positions, stoichiometry, and position relative to the assay surface can all be controlled using this DNA origami scaffold. In the example of FIG. 15, the analyte 116 can bind to the capture antibody 126 which, in turn, is tethered to the DNA origami by complementarity between an oligonucleotide sequence attached to the capture antibody and an oligonucleotide sequence that projects away from the surface on the DNA origami 112b. The second probe 120 which includes a second binding partner 128 and suppressor complementary region 124 can then bind to the analyte and remove suppressor oligonucleotide(s) 118 from the signal region(s) 111 on the first probe 110. It should be understood that the nucleotides designated N can be any nucleotides and should be sufficiently complementary between the capture oligonucleotide 112 and the oligonucleotide conjugated to the capture antibody 126 for stable capture at the temperature and buffer conditions used.

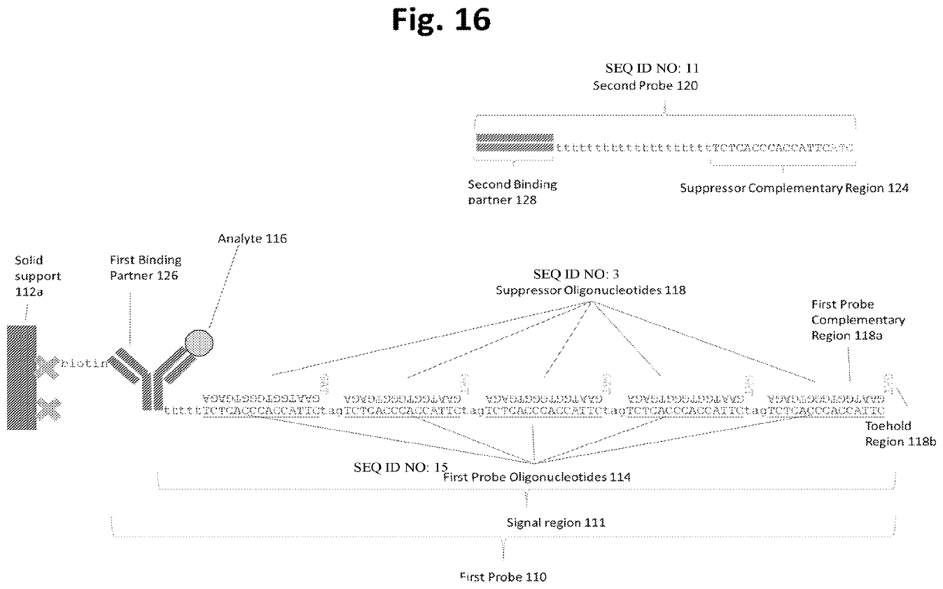

Furthermore, systems can be constructed without the use of an intermediate nanoparticle. For example, one or more signal regions may be covalently or noncovalently bound directly to a capture probe (first analyte binding region) or analyte. For example, FIG. 16 illustrates a system including a first probe 110 in which a signal region 111 is conjugated directly to a capture antibody 126 (first analyte binding region), which is in turn anchored to the assay surface 112a via a biotin-streptavidin interaction. There are many means for conjugating an oligonucleotide or other counter probe to antibodies that will be appreciated by one skilled in the art of biological conjugation chemistry, including those using covalent (e.g. DBCO/azide click chemistry, NHS ester/amine chemistry) or non-covalent chemistry (e.g., biotin-streptavidin interaction). Alternatively, the interaction with the surface could be mediated by the signal region itself, or by the analyte. In the case of nucleic acid analytes, the signal region(s) can be hybridized directly to the analyte at a site proximal to that bound by the second probe; in this instance, the signal region is contiguous with a capture oligonucleotide sequence that interacts stably with the analyte. In the example of FIG. 16, the analyte 116 can bind to the capture antibody 126. Upon binding of the second probe 120 which includes a second binding partner 128 and a suppressor complementary region 124 to the analyte, the second probe can remove one or more suppressor oligonucleotides 118 which each include a first probe complementary region 118a that is complementary to first probe oligonucleotides 114 of the signal region 111 and a toehold region 118b. The second probe can remove the one or more suppressors by TMSD.

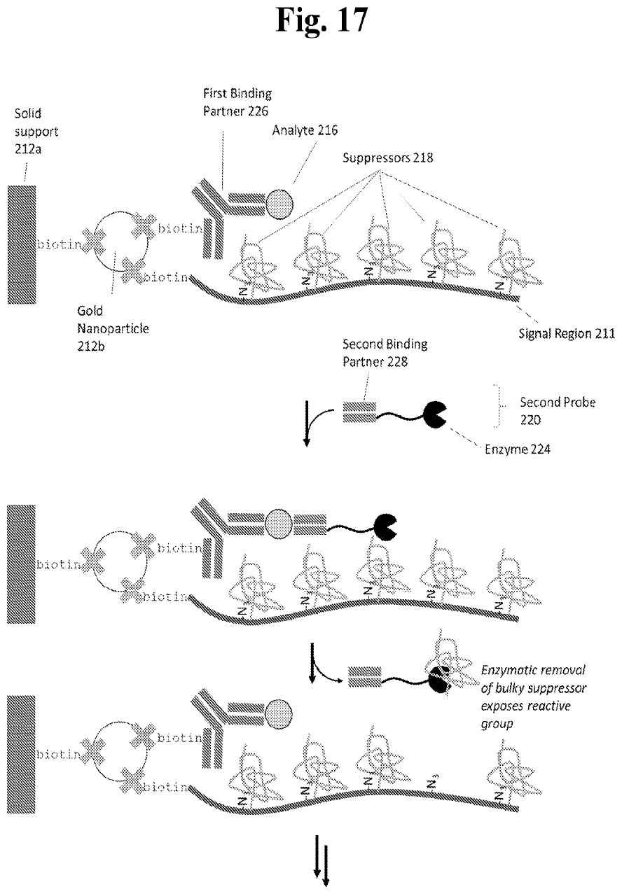

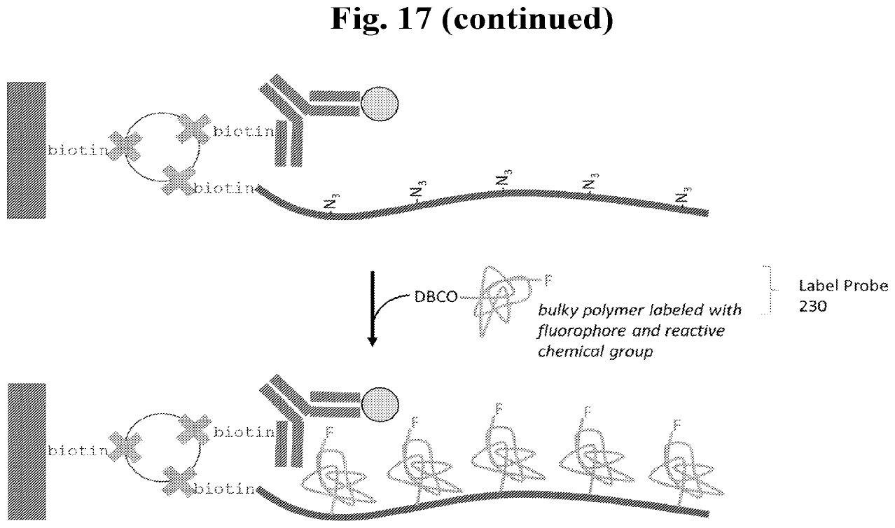

Assay systems can also be constructed using signal regions and/or suppressors that do not comprise nucleic acids, but instead comprise proteins, peptides, other biopolymers, or other chemical groups. One skilled in the arts of biochemistry and biotechnology will appreciate that there are a wide variety of both naturally occurring and engineered enzymes and enzyme substrates, and that the specificity of these enzymes for their substrates is often high enough to be useful in biochemical or biomarker assays. One skilled in the art will also appreciate that peptides and other organic polymers can be engineered (e.g., via methods like solid-phase synthesis and/or enzymatic modification) to bear specific sequences of monomer units and/or site-specific chemical functional groups that can be used for the subsequent attachment of other components in a site-specific manner. It is therefore anticipated that some embodiments of the present approach will utilize first probes, suppressors, and/or second probes that do not comprise nucleic acids. FIG. 17 illustrates an example of one such embodiment that employs an enzyme-conjugated second probe 220 as well as signal region 211 bearing bulky polymer substituents as suppressors 218 that can serve as substrates for the enzyme, and which obscure a reactive chemical group (e.g. N₃) distributed along the length of the signal region 211. The analyte 216 can bind to the first binding partner 226 (first analyte binding region) that is, in turn, bound to a gold nanoparticle 212b which is bound to a solid support 212a by, in this case, streptavidin-biotin interactions. In this embodiment, the binding of the second probe 220 which includes a second binding partner 228 (second analyte binding region) and a suppressor interacting region (enzyme, 224) positions the enzyme 224 in close proximity to the signal region 211, accelerating the degradation and/or removal of a suppressor 218 by the enzyme. Once a suppressor is removed, one or more reactive chemical groups (in this case, an azide functionality) is exposed. After many cycles of second probe binding and dissociation, multiple suppressors will have been degraded and/or removed, exposing many copies of the reactive group. The reactive group may then be reacted with a reagent bearing both a corresponding reactive group (label probe, 230) (in this case, a dibenzocyclooctyne group, DBCO, for copper-free click chemistry) and a detectable label, such as a fluorophore, resulting in the binding of many labels to the exposed sites on the counter probe, yielding an intense signal. For these embodiments, the label-bearing reagent should be relatively non-reactive with sites that are obscured by suppressors; for example, the label may comprise a bulky polymer for which steric hindrance renders it impossible (or very slow) for the reactive group to react with the corresponding reactive group on the signal region when a suppressor is present at an adjacent site on the signal region. It is also preferable for each enzyme to be capable of processing a limited number (e.g., one or two) suppressors before dissociating. This may be accomplished by either using a single-turnover enzyme, an enzyme that dissociates more slowly from its processed substrate than the second probe dissociates from the target, or a so-called “suicide substrate” that inactivates the enzyme upon being processed by the enzyme. To ensure controlled, limited processing by each second probe binding event, it is also beneficial to use suppressors with a single, site-specific processing site, such as a peptide bearing a specific protease substrate sequence or a nucleic acid bearing a nuclease substrate sequence.

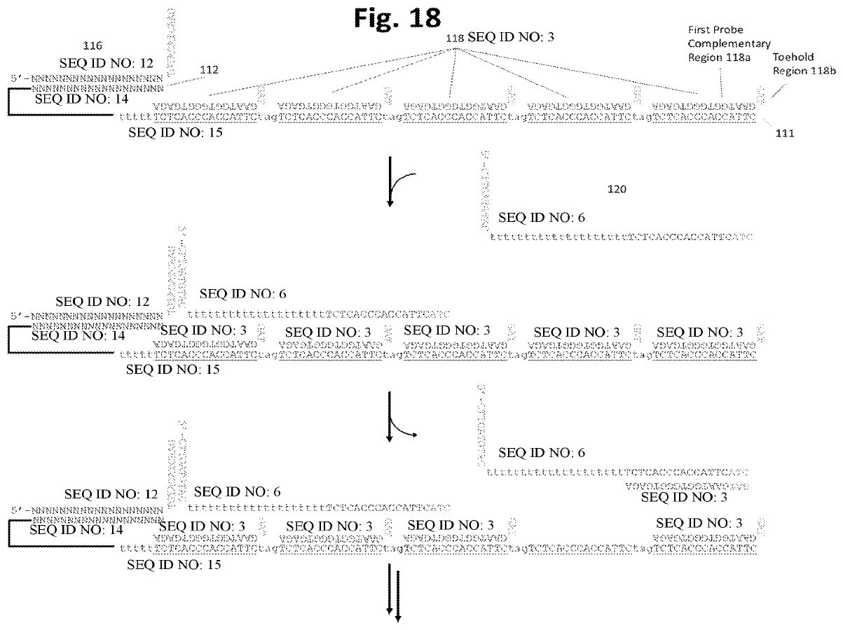

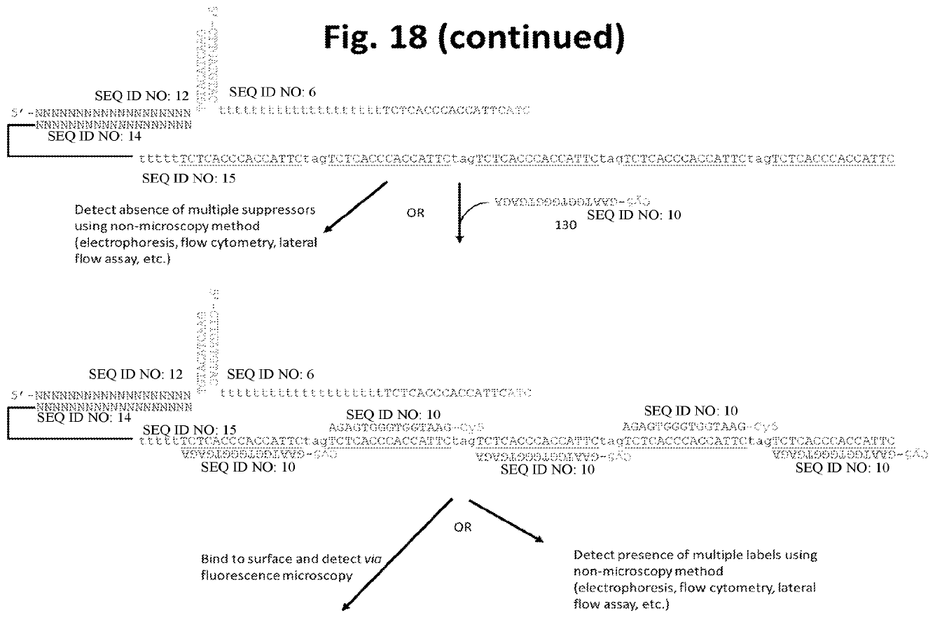

While it is often beneficial to employ an assay surface as a stable anchoring point in order to sensitively detect (e.g., by fluorescence microscopy) first probes that have had multiple suppressor probes removed, this is not a requirement for the presently disclosed methods and systems. For example, the loss of suppressors from first probes, and/or subsequent binding of multiple labels to first probes, can be allowed to occur in solution or colloidal suspension, and then be detected by methods such as polyacrylamide gel electrophoresis (PAGE), agarose gel electrophoresis (AGE), mass spectrometry, flow cytometry, lateral flow assays, or other methods that are sensitive to the loss of multiple suppressors or gain of signal or affinity groups arising from the loss of multiple suppressors (FIG. 18). In some embodiments, the label will comprise a component of high molecular weight that results in a large mobility shift in an electrophoretic assay. As shown in FIG. 18, a first probe can include a capture oligonucleotide 112 which can act as the first analyte binding region to bind the analyte 116. The capture oligonucleotide can be linked to the signal region 111 which can have first probe oligonucleotides that are complementary to suppressor oligonucleotides 118 which can include a toehold region 118b and a first probe complementary region 118a. Upon adding a second probe 120 which can bind to the analyte bound to the first probe, the second probe 120 can remove one or more suppressors 118. After removal of a number of suppressors, the removal of the suppressors can be measured by non-microscopic methods such as electrophoresis, flow cytometry, lateral flow assay, and the like. Alternatively, a label probe 130 can be added which will bind to the signal region 111 where the suppressors 118 have been removed which can be detected likewise by non-microscopic methods such as electrophoresis, flow cytometry, lateral flow assay, and the like or by binding the first probe to a surface and detecting via visual methods such as fluorescence microscopy.

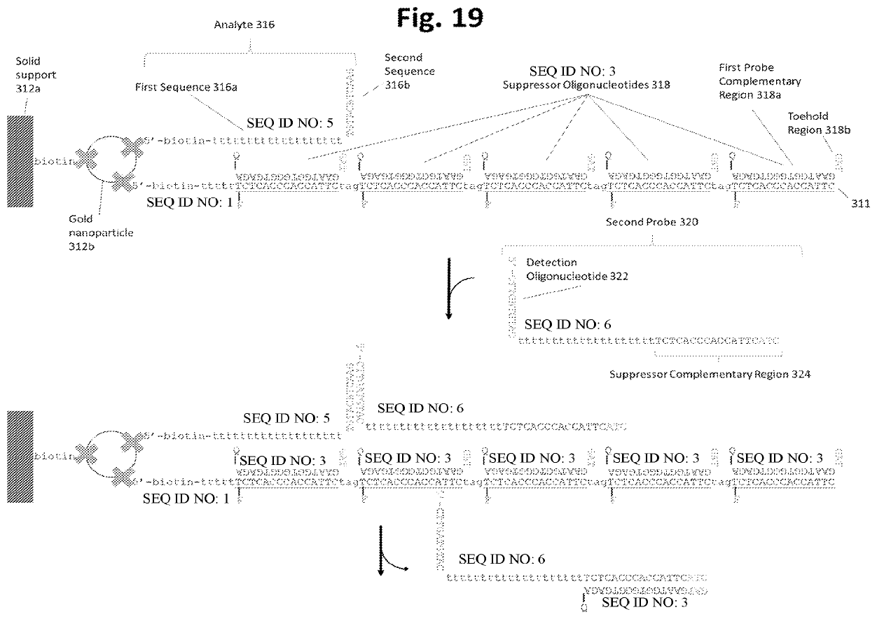

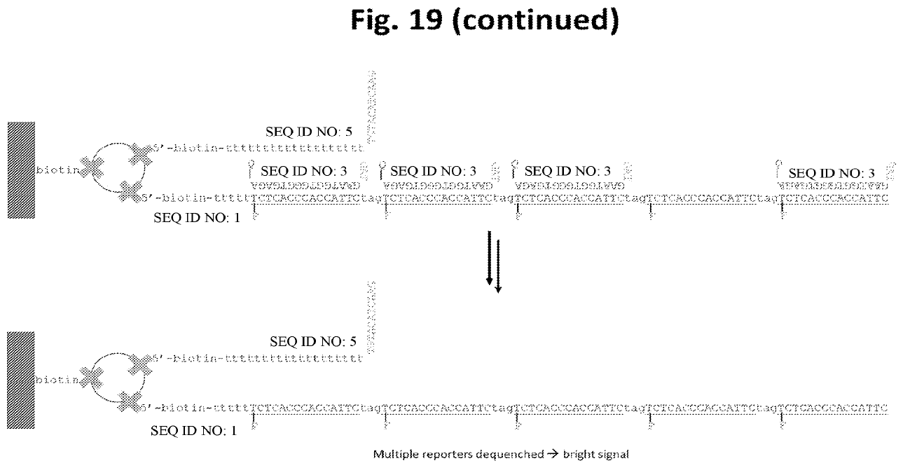

In some embodiments, a label is not added following the removal of suppressors. Instead, the removal of multiple suppressors from a first probe itself serves as the signal that the analyte is, or was, associated with the signal region. For example, the removal of multiple suppressors results in a change of mass and/or hydrodynamic radius that can be detected as, for example, a change in electrophoretic mobility. Alternatively, the signal region may bear detectable labels (e.g., fluorophores) that are rendered detectable upon removal of an adjacent suppressor that bears, for example, a fluorescence quencher. Upon removal of multiple suppressors, multiple fluorophores of the counter probe are dequenched, resulting in a bright fluorescence signal (FIG. 19). As shown in FIG. 19, a first probe can include a signal region 311 that has bound labels, in this case a fluorophore (F), while the suppressors 318 can include a quencher (Q) that blocks fluorescence of the fluorophore when the suppressors are bound to the signal region 311 of the first probe 310. In this example, the analyte 316 is a biotinylated oligonucleotide that can bind to streptavidin on a gold nanoparticle 312b which can be attached to a surface 312a by a streptavidin-biotin interaction and the suppressors 318 include a first probe complementary region 318a and a toehold region 118b. A second probe 320 that includes a detection oligonucleotide 322 (second analyte binding region) and a suppressor complementary region 324 can then bind to the analyte 316 and remove one or more of the suppressors 318, in this case by TMSD. Once the one or more suppressors are removed, signal from the fluorophores can be detected as the quencher(s) are no longer in proximity to the fluorophore(s).

One skilled in the art will appreciate that there are many modifications and features that can be added to a capture oligonucleotide to increase or decrease the stability and/or specificity of analyte capture, or to confer resistance to nucleases and other potentially interfering matrix substances. These potential modification include, but are not limited to: locked nucleic acid (LNA) nucleotides, unlocked nucleic acid (UNA) nucleotides, peptide nucleic acid (PNA) nucleotides, 2′-O-methyl-RNA nucleotides, 2′-fluoro-RNA nucleotides, and phosphorothioate modifications. For example, it is well appreciated in the field that the replacement of several DNA nucleotides in an oligonucleotide probe with LNA residues can increase the stability of hybridization to the analyte, as well as the ability of the probe to displace competing nucleic acids from the analyte.

Further, the system need not involve the biotin-streptavidin interaction, since capture probes, capture antibodies, and/or first probes can be joined to one another directly, or to a nanoparticle, via many other kinds of covalent and non-covalent interactions. For example, the system shown in FIG. 13 employs gold-thiol interactions to bind the nanoparticle to a capture oligonucleotide and to one or more signal regions, as well as to an assay surface.

### Example 6: System and Method Without a Nanoparticle

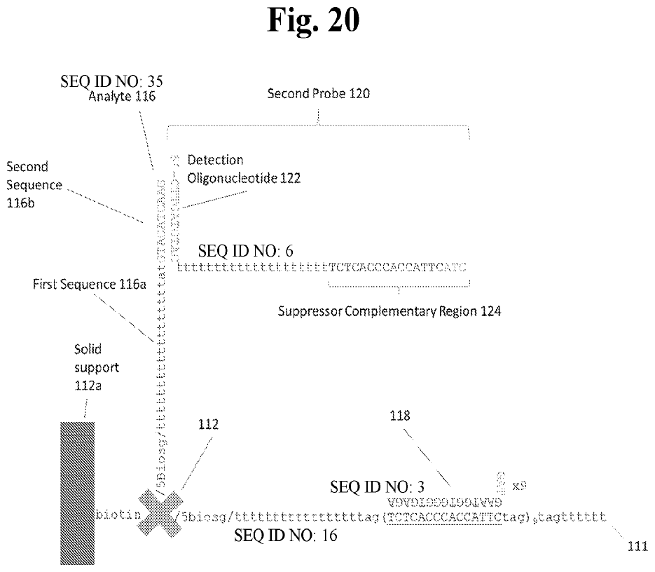

An assay was designed to detect a biotinylated oligonucleotide analyte using a streptavidin-bound signal region 111 that was immobilized on the surface of a biotin-PEG-coated glass coverslip 112a (FIG. 20). In this system, the complex of streptavidin 112, signal region 111, and suppressor oligonucleotides 118 serves as the first probe; the analyte 116 which includes a first sequence 116a and a second sequence 116b binds to the first probe by the streptavidin-biotin interaction; and the second probe 120 includes a detection oligonucleotide 122 linked to a suppressor complementary region 124.

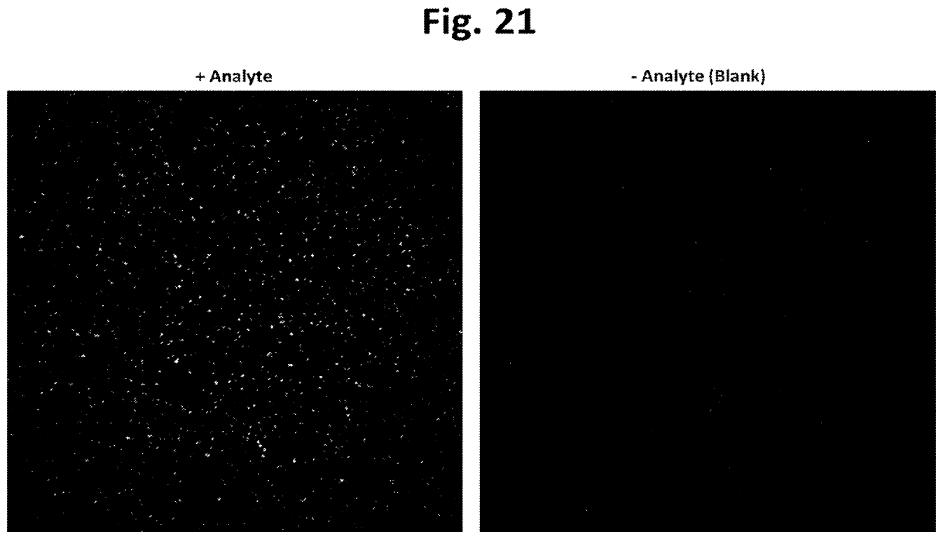

First, a biotin-labeled signal region 111 comprising 9 equivalent binding sites for a complementary suppressor DNA oligonucleotide 118 was combined with a 20-fold molar excess of suppressor 118 in 1×PBS buffer and thermally annealed by heating to 70° C. and then cooled to room temperature for 5 minutes. The complex of signal region 111 and suppressors 118 was then combined with an equimolar (with respect to the concentration of signal region 111) concentration of streptavidin in PBS, and either an equimolar concentration of a biotin-labeled analyte DNA oligonucleotide 116 in PBS or a blank PBS buffer solution. Under these conditions, the majority of streptavidin molecules are expected to be bound to either 1 or 2 copies of the signal region 111, corresponding to 9 or 18 suppressor sites per streptavidin complex. After incubating for 10 minutes, the sample was diluted to 100 pM (with respect to streptavidin) in PBS buffer and added to the surface of a biotin-PEG-coated coverslip and incubated for 10 minutes. The solution was removed, and the second probe 120 was added to the surface at a concentration of 25 nanomolar (nM) in 4×PBS and incubated for 30 minutes at a temperature of approximately 22° C. After the incubation period, the solution of the second probe 120 was removed, and a new solution containing 100 nM of a fluorescently labeled probe oligonucleotide 130 was added. The label probe 130 comprises a DNA sequence complementary to the binding sites for the suppressors 118, and therefore binds to those sites where a copy of a suppressor 118 has been removed. After a 7-minute incubation period at room temperature, the solution of label probe 130 was removed, and a solution comprising an oxygen scavenger system (protocatechuate dioxygenase, 3,4-dihydroxybenzoic acid, and Trolox) in 4×PBS was added. The fluorescence intensity of the surface-bound streptavidin complexes was measured by objective-type total internal reflection fluorescence (TIRF) microscopy with excitation from a 640-nm continuous wave laser and detection using a scientific complementary metal oxide semiconductor (sCMOS) camera. The measurement revealed many bright puncta for the experiment in the presence of analyte, but a lack of equally bright puncta in the experiment performed in the absence of analyte (FIG. 21), indicating that the removal of many copies of suppressors from signal regions bound to a single streptavidin molecule is more efficient in the presence of analyte than in its absence. In parallel, the same experiment was performed using decreasing ratios of analyte to streptavidin, including 0.1:1 and 0.01:1, but keeping the amount of signal region and suppressors in the mixture constant. The lower concentrations of analyte yielded smaller numbers of bright puncta as the concentration of analyte decreased, but still more bright puncta than in the absence of analyte, suggesting that the technique can serve as a method for quantifying the analyte.

One skilled in the art will appreciate that there are many alternatives in addition to streptavidin-biotin and gold-thiol interactions that could accomplish the same coupling, including various click chemistry reagent pairs (e.g., azide+alkyne, or trans-cyclooctene+tetrazine), EDC-NHS coupling between carboxylic acids and amines, thiol-maleimide coupling, amine-NHS ester coupling, electrostatic interactions, coaxial stacking of nucleic acid duplexes, hydrogen bonding interactions, and others.

### Example 7: Solution-based Assay Using Multivalent Capture Probe

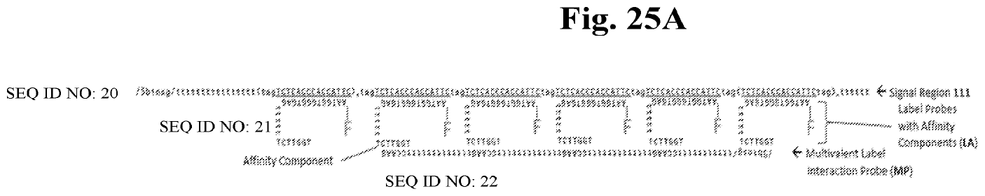

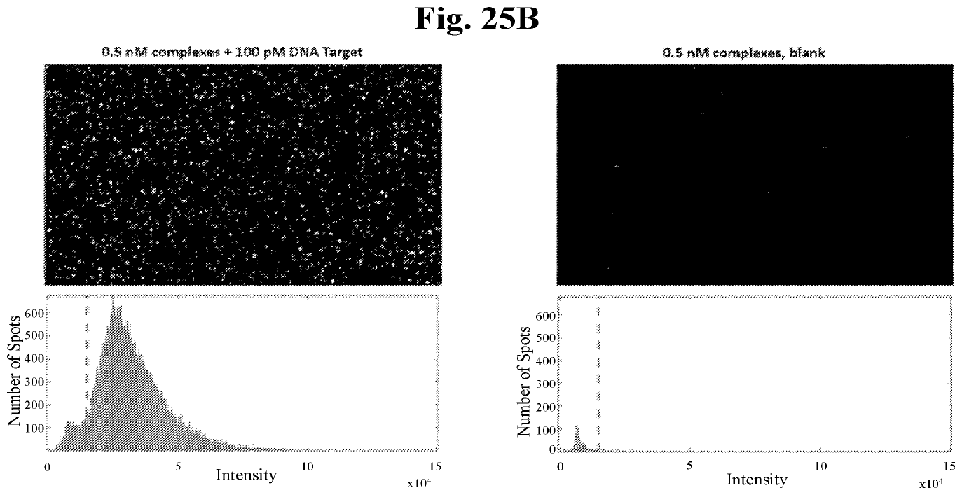

FIG. 24 describes the use of multivalent capture of signal regions of first probes to a solid support by employing a multivalent label interaction probe. Experiments were conducted to demonstrate this approach using the DNA analyte 116 (DNA target) and signal region 111 connected by a streptavidin bridge as described in Example 6. However, unlike Example 6, the incubation steps involving the removal of suppressor oligonucleotides and the binding of label probes were performed in solution. The label probes in these experiments (FIG. 25, panel A) comprised a fluorophore (F, e.g., Alexa Fluor 647) as well as an Affinity Component comprising a 5-nucleotide motif (CTTGG) that interacts with a complementary sequence in the Multivalent Label Interaction Probe (MP). While the interaction of an individual Affinity Component with MP is not strong enough under ambient conditions (approximately 25° C., approximately 150-600 mM Na⁺) to yield a stable complex, the presence of multiple LA probes on a single signal region yield a multivalent interaction with MP that is stable under ambient conditions, permitting the immobilization of the signal region to a solid support (e.g., a biotinylated, streptavidin-modified glass coverslip) for detection by fluorescence microscopy.

First, a biotin-PEG-coated coverslip was coated with 1 mg/mL streptavidin for 10 minutes, washed three times with 1×PBS buffer, coated with 100 nM of Multivalent Label Interaction Probe (MP) for 30 min, and washed three times with 1×PBS. This yielded a solid support coated with MP for multivalent capture of signal regions. A biotin-labeled signal region 111 comprising 9 equivalent binding sites for a complementary suppressor oligonucleotide 118 was combined with a 20-fold molar excess of suppressor 118 in 1×PBS buffer and thermally annealed by heating to 70° C. and then cooled to room temperature for 5 minutes. The complex of signal region 111 and suppressors 118 was then combined with a threefold lower (with respect to the concentration of signal region 111) molar concentration of streptavidin in PBS and incubated for 10 min, then diluted to 1 nM (with respect to streptavidin) in 4×PBS that either contained 200 pM biotin-labeled analyte oligonucleotide 116 or did not contain any oligonucleotide (blank). Next, the solutions containing the complexes of signal regions, suppressors, analyte or blank solution, and streptavidin were combined with equal volumes of 50 nM second probe 120 in 4×PBS, yielding concentrations of 25 nM second probe, 0.5 nM streptavidin complex, and 100 or 0 nM of analyte oligonucleotide. These solutions were incubated at approximately 25° C. for 2 hours. After the incubation period, each solution was combined with an equal volume of another solution containing 50 nM of a fluorescently labeled probe oligonucleotide with affinity component (LA) in 1×PBS (i.e., LA present at a twofold molar excess over 120). Each solution was added to a separate chamber exposed to the coverslip coated with MP, and incubated for 30 min at room temperature, protected from the light, to allow for multivalent capture of complexes bearing multiple copies of LA. The solutions were removed, and replaced by a solution comprising an oxygen scavenger system (protocatechuate dioxygenase, 3,4-dihydroxybenzoic acid, and Trolox) in 4×PBS. The fluorescence intensity of the surface-bound streptavidin complexes was measured by objective-type total internal reflection fluorescence (TIRF) microscopy with excitation from a 640-nm continuous wave laser and detection using a scientific complementary metal oxide semiconductor (sCMOS) camera. The measurement revealed many bright puncta in the reactions containing the DNA analyte, but few or no bright puncta in the reactions containing a blank PBS solution with no analyte (FIG. 25, panel B). Thus, detection of an analyte by performing the reaction with the second probe in solution followed by multivalent capture at an imaging surface is feasible.

### Example 8: Branched Signal Region with 18 Label Probe Sites Provides Brighter Positive Signal than a Linear Signal Region with 9 Label Probe Sites

Experiments were conducted to demonstrate the utility of a signal region that comprises a branched nucleic acid structure rather than a linear structure. That is, instead of a linear array of suppressor oligonucleotide binding sites, the first probe may comprise a branched structure of nucleic acids where the suppressor oligonucleotide binding sites are distributed among different branches of the structure. One exemplary experiment was performed identically to Example 6 except that in some measurements the linear signal region 111 was replaced with a branched structure B as shown in FIG. 26. Use of the branched structure provides the opportunity for each target (analyte) molecule to, through its interaction with a plurality of second probes, remove a larger number of suppressor oligonucleotides (up to 18) than in the case of the linear first probe (up to 9) while keeping the suppressor sites relatively close and accessible to the analyte or analyte binding site. Use of the branched first probe B resulted in an approximately 1.5-fold increase in average brightness of fluorescent puncta (FIG. 26, panel D) compared to the linear signal region 111 (FIG. 26, panel C) in experiments conducted in the presence of the analyte oligonucleotide 116, while not significantly increasing the brightness of puncta in the presence of a blank solution.

### Example 9: Detection of a Single-Nucleotide Substitution with ≥99.99% Specificity

An assay was designed to detect a target DNA sequence in the presence of a non-target sequence that is identical to the target sequence except for a single adenosine-to-thymine (A to T) substitution. The target sequence T1 (SEQ ID NO: 17) is the same as analyte 116 disclosed in FIG. 20 (5′biotin-TTTTTTTTTTTTTTTTTTATGTACATCAAG), and the non-target sequence T2 is 5′biotin-TTTTTTTTTTTTTTTTTTATGTACTTCAAG (SEQ ID NO: 35), where the position of the single-nucleotide substitution is shown in bold and underlined in both sequences. The experiment was conducted identically to that in Example 6 except for the differences discussed below.

In addition to the target DNA T1 and no-DNA control conditions (blank, where the target DNA is omitted), replicate measurements were performed wherein the target DNA was replaced by a non-target DNA T2. T1 and T2 were used at the same concentration in their respective samples (200 picomolar), enabling a direct comparison between measurements of samples containing these two oligonucleotides.

In some replicates, a competitor sequence C (5′-CTTGAAGTAC) (SEQ ID NO: 36) designed to be fully complementary to a portion of T2, but to have a single-nucleotide mismatch to the corresponding portion of T1, was added at a concentration of approximately 100 nanomolar during the incubation step with 25 nanomolar of the second probe 120.

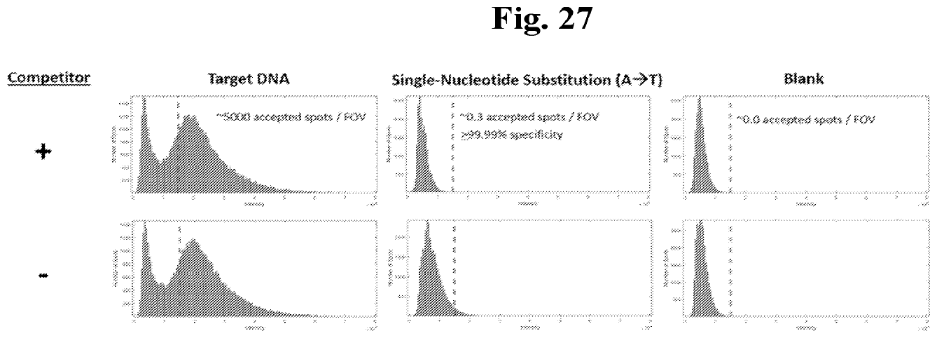

As shown in FIG. 27, histograms of the intensity of fluorescent puncta observed by fluorescence microscopy are consistent with the expected outcomes: namely, a population of puncta were observed in the presence of T1 that were much (approximately 4- to 10-fold) brighter than puncta observed in the presence of T2 or the blank sample, and the addition of the competitor C further reduced the average intensity of puncta in the presence of T2 such that the histogram resembles the blank sample very closely. This intensity difference was great enough that a threshold (dashed vertical line in FIG. 27) could be used to exclude nearly all of the spots in the blank and T2 datasets while counting (or accepting) the majority of bright spots as detection events of T1 in the presence of T1. In the presence of the competitor, the ratio of puncta whose intensity exceeds the threshold in the presence of T1 (approximately 5000 per field of view, FOV) to those whose intensity exceeds the threshold in the presence of T2 (approximately 0.3 per FOV) suggests a single-nucleotide selectivity greater than 99.99%.

### Example 10: Assay of a Target Nucleic Acid Analyte where the Signal Regions are Associated with the Analyte by Direct Hybridization to the Analyte Sequence

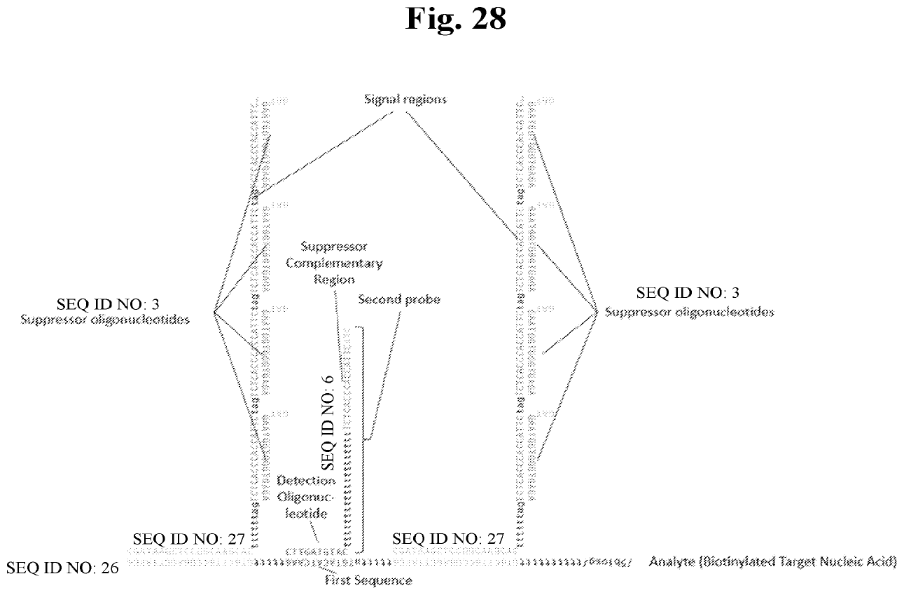

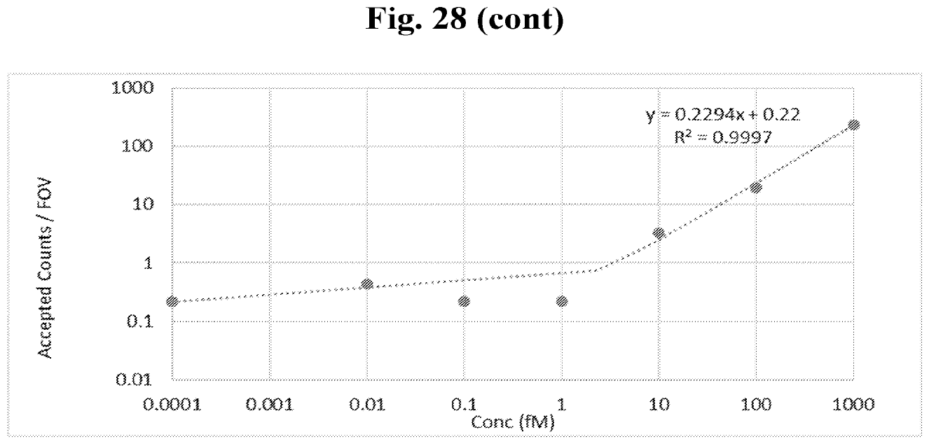

An assay was designed to detect a biotinylated oligonucleotide analyte using a signal region that hybridized directly to the analyte (FIG. 28), where the complex of the signal region and analyte was immobilized on the surface of a streptavidin-coated glass coverslip. First, a biotin-PEG-coated coverslip was incubated with 1 mg/mL streptavidin for 10 min, then washed 3 times with 1×PBS, to coat the coverslip with streptavidin. Next, solutions containing between 0 and 1000 femtomolar (fM) of the Analyte, 10 nM of each of two Signal region oligonucleotide sequences (FIG. 28), and 100 nM of Suppressor oligonucleotide (FIG. 28) in 1×PBS were heated to 70° C. for 1 min and then placed at room temperature (˜22° C.) for 10 minutes. The solutions of 0 to 1000 fM Analyte were incubated in separate chambers in contact with the streptavidin-coated coverslip for approximately 30 min at 25° C. to allow for capture of the Analyte complexes at the coverslip surface. Next, the analyte solution was removed and replaced by a solution containing 25 nM of the Second Probe in 4×PBS, and the coverslip assembly was incubated at 25° C. for 1 h and 30 min. The Second Probe solution was removed and replaced by a solution containing 100 nM of Label probe 130 in 4×PBS, and incubated for 5 min at approximately 22° C. The Label probe solution was removed and replaced by a solution comprising an oxygen scavenger system (protocatechuate dioxygenase, 3,4-dihydroxybenzoic acid, and Trolox) in 4×PBS. The fluorescence intensity of the surface-bound Signal region complexes was measured by objective-type total internal reflection fluorescence (TIRF) microscopy with excitation from a 640-nm continuous wave laser and detection using a scientific complementary metal oxide semiconductor (sCMOS) camera. The number of puncta per microscope field of view with fluorescence intensities between 10,000 AU and 50,000 AU—thresholds chosen to minimize counting of false positives in blanks but maximize counting of true positives in the presence of the Analyte—was quantified for each condition ranging from 0 to 1000 fM Analyte. The number of spots passing these intensity thresholds (Accepted Counts) per FOV was plotted as a function of concentration (FIG. 28). The limit of detection of this assay, determined as the concentration predicted to give a number of Accepted Counts equal to three standard deviations above the blank signal, was 2 fM. Thus, the assay gives at least low-femtomolar sensitivity for detection of a nucleic acid analyte.

### Example 11: Detection of a Biotinylated Nucleic Acid Analyte and Single-Nucleotide Discrimination in an Assay where Streptavidin-Coated Microparticles are Used as the Capture Surface

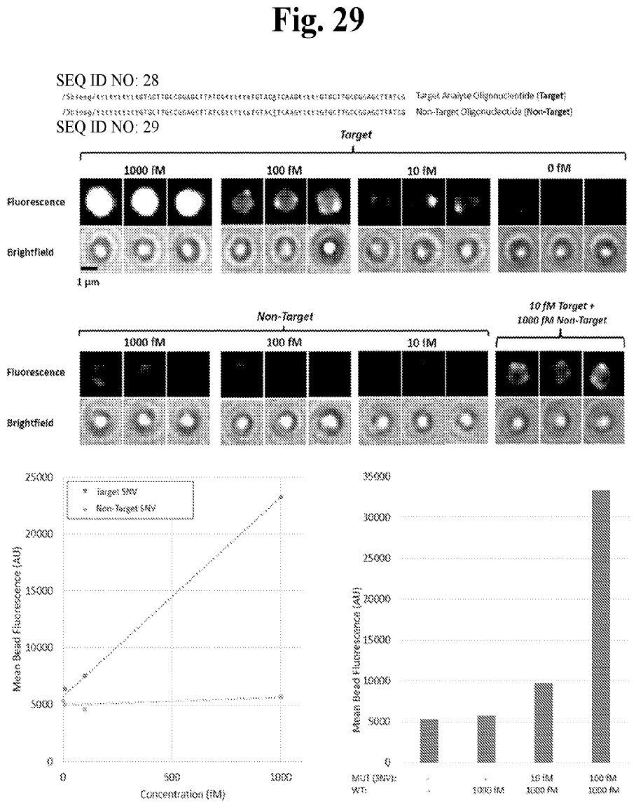

An assay was designed to detect a biotinylated target oligonucleotide (Target) but not a non-target oligonucleotide (Non-Target), where the target and non-target differ by a single-nucleotide substitution (underlined nucleotide in FIG. 29)—i.e., the Target and Non-Target are single-nucleotide variants or point mutations of one another—where the Target and/or Non-Target are immobilized at the surface of a streptavidin-coated microparticle rather than to a microscope coverslip. The assay uses the same Signal region, Suppressor, and Second probe oligonucleotides as in Example 10 (FIG. 28), and the Target oligonucleotide is the same as the Analyte in Example 10 (FIG. 28). In addition, a competitor oligonucleotide C (5′-CTTGAAGTAC) (SEQ ID NO: 36) was used. First, solutions containing between 0 and 1000 femtomolar (fM) of the Target and/or Non-Target, 10 nM of each of two Signal region oligonucleotide sequences (FIG. 28), and 100 nM of Suppressor oligonucleotide (FIG. 28) in 1×PBS were heated to 70° C. for 1 min and then placed at room temperature (˜22° C.) for 10 minutes. Next, the samples were added to suspensions of DynaBeads MyOne T1 magnetic streptavidin-coated beads (microparticles) to allow the biotinylated oligonucleotides to bind to the beads for 1 h 30 min. The tubes were placed on a magnetic rack to pull down the magnetic beads, and the supernatant was removed from each tube, leaving the beads in the tube. The beads were resuspended in a solution containing 25 nM of the Second Probe in 4×PBS, and incubated at 25° C. for 1 h and 30 min. The beads were pulled down on a magnetic tube rack. The Second Probe solution was removed and the beads resuspended in a solution containing 100 nM of Label probe 130 in 4×PBS, and then incubated for 10 min at approximately 22° C. The beads were pulled down on a magnetic tube rack. The Label probe solution was removed and the beads resuspended in a solution comprising an oxygen scavenger system (protocatechuate dioxygenase, 3,4-dihydroxybenzoic acid, and Trolox) in 4×PBS. The suspensions of beads were pulled down via magnet onto a biotin-PEG-coated coverslip, which immobilized the beads via unoccupied streptavidin sites. The surface-bound beads were imaged by objective-type highly inclined laminated optical sheet (HILO) microscopy with excitation from a 640-nm continuous wave laser and detection using a scientific complementary metal oxide semiconductor (sCMOS) camera. Each set of beads was also imaged using bright-field microscopy to determine the positions of beads independent of fluorescence intensity. The fluorescence intensity of each bead was determined by summing the fluorescence over the entire bead area after subtracting any background fluorescence using a 20-pixel rolling-ball radius in ImageJ, and the average bead intensity within each field of view was calculated. As shown in FIG. 29, fluorescence signal was detectable in samples containing as little as 10 fM Target, but little or no signal above background was detected in samples containing 10-1000 fM Non-Target. In addition, 10 fM Target was easily detectable in a solution also containing 1000 fM Non-Target. The limit of detection, determined as the concentration of Target expected to yield a signal 3 standard deviations above the blank signal, was approximately 1 fM. Thus, low-femtomolar detection of a target nucleic acid, and the ability to distinguish between single-nucleotide variants or mutants with high specificity, was demonstrated using a bead-based assay.

### Example 12: Detection of a Biotinylated Oligonucleotide in 25% Serum

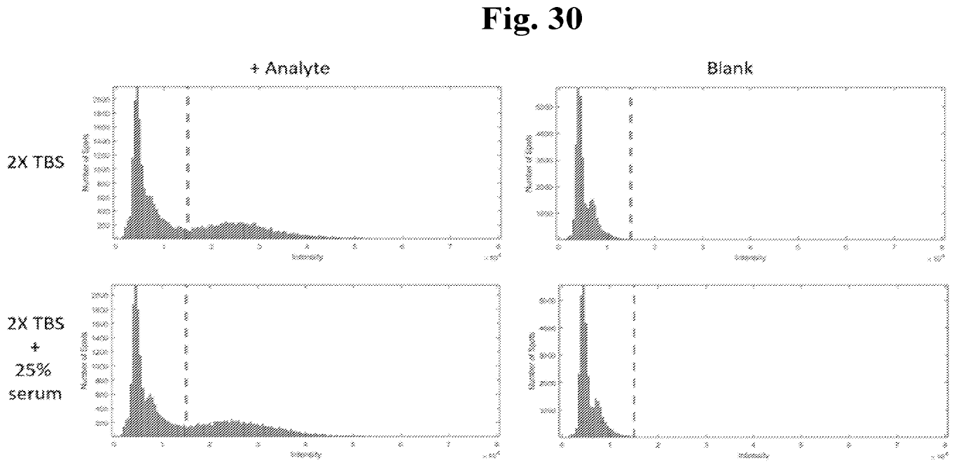

An assay was designed to detect a biotinylated target oligonucleotide (Analyte) in 25% serum. The assay was identical to that of Example 6, except that the incubation with the Second Probe was performed in 2X TBS with or without 25% horse serum. Referring now to FIG. 30, The assay yielded statistically indistinguishable numbers of bright fluorescent puncta exceeding a threshold of 20,000 AU in the presence (+Analyte: 1198+/−16, Blank: 0.8+/−0.3, error bars=1 s.d.) and absence (+ Analyte: 1202+/−21, Blank: 0.5+/−0.3, error bars=1 s.d.) of 25% serum—i.e., results were within one standard deviation in the presence and absence of serum—showing that the assay is robust to a complex biological matrix.

### Example 13: Detection of a Target Oligonucleotide Carrying the Natural EGFR T790M Mutation

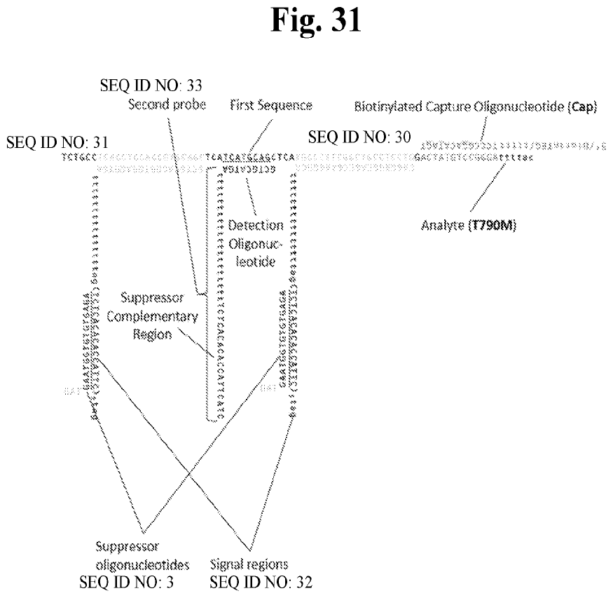

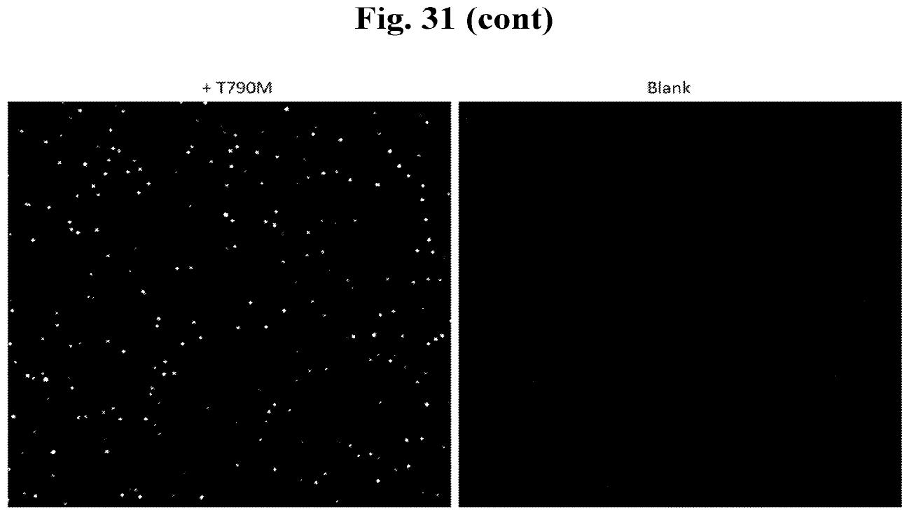

An assay was designed to detect a target oligonucleotide analyte comprising the naturally occurring EGFR T790M mutation, a biomarker for cancer. The assay was performed in a manner similar to Example 10, but with a different set of oligonucleotide sequences as shown in FIG. 31. In addition, instead of capturing the target oligonucleotide sequence via a biotin label, the analyte sequence is captured using a biotinylated capture oligonucleotide (Cap) comprising several locked nucleic acid (LNA) modifications (underlined oligonucleotides in Cap, FIG. 31). First, a biotin-PEG-coated coverslip was incubated with 1 mg/mL streptavidin for 10 min, then washed 3 times with 1×PBS, to coat the coverslip with streptavidin. The streptavidin-modified surface was further coated with Cap by incubating with a 100 nM solution of Cap in 1×PBS for 30 min, followed by washing 3 times in 1×PBS. Next, solutions containing either 0 or 10 pM of the Analyte (T790M), 10 nM of each of two Signal region oligonucleotide sequences (see Example 10 and FIG. 28), and 100 nM of Suppressor oligonucleotide (see Example 10 and FIG. 28) in 1×PBS were heated to 70° C. for 1 min and then placed at room temperature (˜22° C.) for 10 minutes. The solutions of 0 or 10 pM analyte were incubated in separate chambers in contact with the Cap-coated coverslip for approximately 30 min at 25° C. to allow for capture of the analyte complexes at the coverslip surface. Next, the analyte solution was removed and replaced by a solution containing 25 nM of the Second Probe in 4×PBS, and the coverslip assembly was incubated at 25° C. for 1 h and 30 min. The Second Probe solution was removed and replaced by a solution containing 100 nM of Label probe (5′-Alexa Fluor 647-AATGGTGTGTGAG-3′) (SEQ ID NO: 38) in 4×PBS, and incubated for 10 min at approximately 22° C. The Label probe solution was removed and replaced by a solution comprising an oxygen scavenger system (protocatechuate dioxygenase, 3,4-dihydroxybenzoic acid, and Trolox) in 4×PBS. The fluorescence intensity of the surface-bound Signal region complexes was measured by objective-type total internal reflection fluorescence (TIRF) microscopy with excitation from a 640-nm continuous wave laser and detection using a scientific complementary metal oxide semiconductor (sCMOS) camera. Referring now to FIG. 31, hundreds of bright puncta per field of view were visible in the presence of 10 pM analyte, but few or no bright puncta were evident in the Blank measurements lacking the analyte. Thus, the assay can detect a sequence comprising the EGFR T790M mutation.

### Example 14: Detection of a Target Protein Analyte (Antigen) Using a First Probe Comprising a Capture Antibody and a Signal Region Joined by Streptavidin-Biotin and a Planar Surface

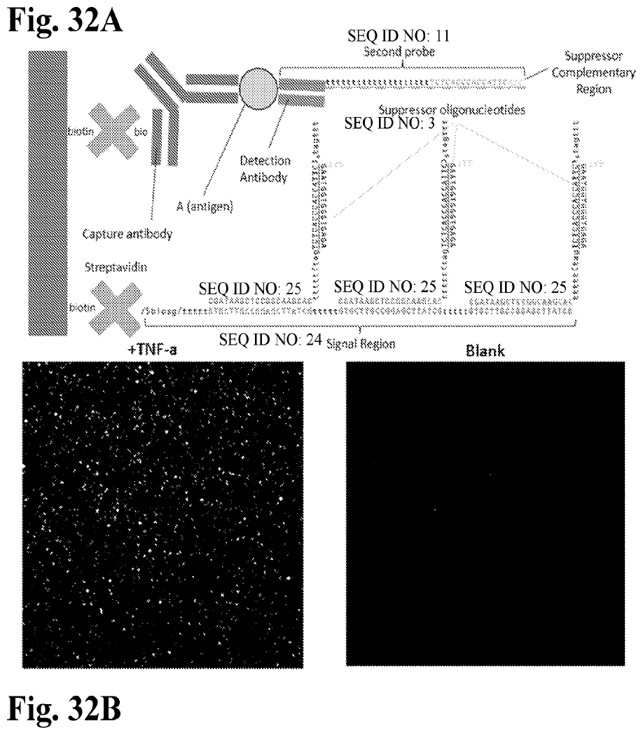

An assay was designed to detect a target protein analyte (antigen, A), TNF-alpha, using a first probe comprising: a biotinylated capture antibody with affinity for TNF-alpha; a biotinylated, branched signal region; and a biotinylated, streptavidin-coated surface to link the capture antibody and signal region (FIG. 32). The second probe comprises a detection antibody conjugated to a DNA oligonucleotide comprising a suppressor complementary region. First, a biotin-PEG-coated coverslip was incubated with 1 mg/mL streptavidin for 10 min, then washed 3 times with 1×PBS, to coat the coverslip with streptavidin. Next, the coverslip was incubated with a mixture containing a 1:10, 1:100, 1:1000, or 1:10,000 molar ratio of biotinylated capture antibody to biotinylated signal region, each solution comprising approximately 100 nanomolar of biotinylated capture antibody. After a 30-min incubation, the solution was removed and the surface washed 3 times with 1×PBS. Next, a solution containing either 10 or 0 ng/ml TNF-alpha in 10 mg/mL and 1×PBS was added to the surface and incubated for 1 hour at 25° C. These solutions were removed and replaced by solutions containing 25 nanomolar of the second probe in 1×TBS (1× Tris-buffered saline, pH 8.0) and incubated at 25° C. for 1 h 30 min in a humid environment. The second probe solution was removed and replaced by a solution containing 100 nanomolar of Label probe 130 in 4×PBS buffer, and incubated for 10 min at approximately 22° C. The Label probe solution was removed and replaced by a solution comprising an oxygen scavenger system (protocatechuate dioxygenase, 3,4-dihydroxybenzoic acid, and Trolox) in 4×PBS. The fluorescence intensity of the surface-bound Signal region complexes was measured by objective-type total internal reflection fluorescence (TIRF) microscopy with excitation from a 640-nm continuous wave laser and detection using a scientific complementary metal oxide semiconductor (sCMOS) camera. Several hundred bright puncta were observed within each field of view for the assay performed in the presence of TNF-alpha, but few or no equally bright puncta were observed in the absence of the analyte (FIG. 32), demonstrating the feasibility of detecting an analyte using this first probe configuration.

Therefore, the present invention is well adapted to attain the ends and advantages mentioned as well as those that are inherent therein. While numerous changes may be made by those skilled in the art, such changes are encompassed within the spirit of this invention as illustrated, in part, by the appended claims.

The foregoing description of specific embodiments of the present disclosure has been presented for purpose of illustration and description. The exemplary embodiments were chosen and described in order to best explain the principles of the disclosure and its practical application, to thereby enable others skilled in the art to best utilize the subject matter and various embodiments with various modifications are suited to the particular use contemplated. Different features and disclosures of the various embodiments within the present disclosure may be combined within the scope of the present disclosure.

It should be understood throughout this disclosure that the first probes disclosed can have one or more signal regions and one or more first analyte binding regions. By way of example, but not limitation, a nanoparticle can be bound to multiple signal regions and/or multiple first analyte binding regions. Furthermore, the first analyte binding regions and signal regions may be parts of a common molecule or may be separate molecules in proximity sufficient to permit association of the second probes with the suppressors bound to the first probe with both the first analyte binding region and second analyte binding region are associated with the analyte.

While the embodiments described and illustrated above have employed common elements due to the convenience and economy of constructing various systems from shared materials, the approaches described herein need not employ these specific materials.

1.-210. (canceled)

211. A method of detecting an analyte in a sample, the method comprising:
(a) mixing a sample with:
(i) a plurality of first probes, each first probe comprising an analyte binding partner and a signal region, wherein the analyte binding partner comprises a first analyte binding region having affinity to the analyte; and wherein the first analyte binding region and the signal region are either directly associated as part of a common molecule or indirectly associated as two distinct molecules;
(ii) a plurality of label probes, wherein each label probe is able to associate with the signal region;
(iii) a plurality of suppressors, wherein each suppressor is able to associate with the signal region; and wherein the suppressors prevent detection of the label probes when the suppressors are associated with the signal region or wherein the suppressors prevent association of the label probes with the signal region; and
(iv) a plurality of second probes, each second probe comprising a second analyte binding region and a suppressor interacting region, wherein the second analyte binding region has affinity to the analyte; wherein the suppressor interacting region is indirectly or directly capable of binding to, removing, or degrading one or more of the suppressors; and wherein the interaction of the first analyte binding region to the analyte is characterized by a rate constant of dissociation that is slower than the rate constant of dissociation between the second analyte binding region and the analyte;
to provide a reaction mixture;
(b) incubating the reaction mixture under conditions sufficient for the analyte to bind to the first analyte binding region;
(c) incubating the reaction mixture under conditions sufficient for the second probe to bind to and dissociate from the analyte and remove one or more suppressors from the signal region of the plurality of first probes or degrade one or more suppressors;
(d) incubating the reaction mixture under conditions sufficient to permit the label probe to associate with the signal regions of first probes that have had one or more suppressors removed or degraded; and
(g) measuring a signal generated from the label probes associated with the signal regions of the first probe to determine the presence of the analyte in the sample.

212. The method of claim 211, wherein each of the analyte binding partners is directly or indirectly associated with a first probe.

213. The method of claim 211, wherein the first probes comprise a nucleic acid, an antibody or antigen-binding antibody fragment, or an aptamer.

214. The method of claim 211, wherein the second probes comprise a nucleic acid, an antibody or antigen-binding antibody fragment, or an aptamer.

215. The method of claim 211, wherein the analyte binding partners and first probes are bound to one or more microparticles, and wherein at least one of the first probes is within approximately 100 nm from at least one of the analyte binding partners on each microparticle.

216. The method of claim 215, wherein each of the analyte binding partners is immobilized on the one or more microparticles within approximately 20 nm of at least one of the first probes.

217. The method of claim 215, wherein each microparticle has a diameter from approximately 1 micron to approximately 10 microns.

218. The method of claim 215, wherein the microparticle is a bead.

219. The method of claim 211, wherein the affinity between the first analyte binding region and the analyte is characterized by a first rate constant of dissociation, the affinity between the second analyte binding region and the analyte is characterized by a second rate constant of dissociation, and the first rate constant of dissociation is at least ten-fold slower than the second rate constant of dissociation.

220. The method of claim 211, wherein the analyte comprises a nucleic acid.

221. The method of claim 220, wherein the first analyte binding region comprises a first capture oligonucleotide, wherein the first capture oligonucleotide has a sequence that is complementary to a first sequence of the analyte, wherein the second analyte binding region comprises a detection oligonucleotide, wherein the detection oligonucleotide has a sequence that is complementary to a second sequence of the analyte.

222. The method of claim 220, wherein the signal region comprises a first probe oligonucleotide, and wherein the suppressor binding regions each comprise a common sequence.

223. The method of claim 211, wherein the analyte comprises a polypeptide.

224. The method of claim 223, wherein the analyte binding partner comprises a first antibody or antigen-binding antibody fragment, a first aptamer, or a first ligand of the analyte; wherein the second analyte binding region comprises a second binding partner with affinity to the analyte; wherein the suppressor interacting region is linked to the second binding partner; and wherein the second binding partner is a second antibody or antigen-binding antibody fragment, a second aptamer, or a second ligand of the analyte

225. The method of claim 211, wherein the number of second probes is in excess of the number of suppressors.

226. The method of claim 211, wherein the number of second probes is in excess of the number of first probes.

227. The method of claim 211, wherein the number of label probes is in excess of the number of second probes.

228. The method of claim 211, wherein the label probes comprise a signaling component.

229. The method of claim 228, wherein the signaling component is selected from the group consisting of a fluorophore, a fluorogenic compound, a fluorescent protein, a small organic fluorophore, and a chemiluminescence-generating enzyme.
